(19) United States
(12) Patent Application Publication
(10) Pub. No.: US 2023/0227925 A1
(43) Pub. Date: Jul. 20, 2023

(54) NANOPARTICLE PROBES AND THEIR USE IN NUCLEIC ACID DETECTION

(71) Applicants: INDICATE SOLUTIONS, S.L., Donostia-San Sebastian (ES); FUNDACION DONOSTIA INTERNATIONAL PHYSICS CENTER, Donostia-San Sebastián (ES); ADMINISTRACIÓN GENERAL DE LA COMUNIDAD AUTÓNOMA DE EUSKADI, Vitoria-Gasteiz (Araba/Álava) (ES)

(72) Inventors: Charles Henderson LAWRIE, Donostia-San Sebastián (ES); Marek GRZELCZAK, Donostia-San Sebastián (ES); María Sanromán IGLESIAS, Donostia-San Sebastián (ES)

(73) Assignee: INDICATE BIOTECHNOLOGY (SHANGHAI) CO., LTD., Shanghai (CN)

(21) Appl. No.: 18/009,329

(22) PCT Filed: Jun. 9, 2022
(86) PCT No.: PCT/EP2021/065536

§ 371 Date: Dec. 9, 2022

(30) Foreign Application Priority Data
Jun. 9, 2020 (EP) 20382499.0

(51) Int. Cl. C12Q 1/70; C12Q 1/6834; G01N 21/78

(52) U.S. Cl. CPC C12Q 1/701; C12Q 1/6834; G01N 21/78

(57) ABSTRACT

The invention provides a method for detecting the presence of a target nucleic acid analyte, for example a pathogen or virus nucleic acid, in a sample using oligonucleotide probe-functionalised nanoparticles, where hybridisation of at least three different oligonucleotide probes to at least three different target sequences in the target analyte causes agglomeration of the nanoparticles and a visible colour change. The invention also provides a population of such oligonucleotide probe-functionalised nanoparticles and a related kit for detection of a target nucleic acid analyte.

This application claims priority from EP20382499.0 filed 9 Jun. 2020, the contents and elements of which are herein incorporated by reference for all purposes.

### FIELD OF THE INVENTION

The present invention relates to detection of target nucleic acid, particularly to detection of the nucleic acid of a microorganism or a virus such as SARS-CoV-2.

### Background to the Invention

COVID-19

COVID-19 is a severe respiratory disease caused by the Coronavirus severe acute respiratory syndrome coronavirus 2 (SARS-CoV-2). It was first identified in Wuhan, the capital of China's Hubei province in December of 2019. It spread quickly from its origin to take hold globally resulting in the current pandemic that has resulted in tens of thousands of deaths, hundreds of thousands, if not millions, of infected individuals and caused universal disruption and global economic recession.

The key to successful control of an emerging epidemic or pandemic is the identification and isolation of infected individuals, whether symptomatic or asymptomatic. With the development and establishment of a pandemic, in order to ensure the continued functioning of society, individuals that have already been infected, but may not be aware either because of mild, confusing or no symptoms, need to be identified as those that have developed immunity through the development and subsequent detection of specific antibodies against the disease. In the absence of an effective vaccine or treatment, testing is at the front-line of infection control.

There are currently three major types of SARS-CoV-2 test that exist (antigenic, antibody and genetic) with very different properties, advantages and disadvantages. Antigenic tests detect parts of the virus (usually proteins) and are the most common form of rapid tests employed at the moment. However, due to cross-reactivity with other related proteins these tests tend to be much less accurate than genetic tests. Antibody tests are also rapid tests that detect the presence of antibodies (either IgG or IgM) against the virus, and are generally more specific than antigen tests but do not detect patients when they are most contagious as antibodies do not appear in persons until 10-14 days after infection. The gold standard of viral testing remains the genetic test that detects the specific nucleic acid (RNA) sequence of the virus. At the moment all genetic tests on the market are based on PCR. Even when incremental improvements are made to the turn-around time of this technology PCR-based tests are limited in their scalability as they require specialised laboratories, expensive equipment, reagents and trained personnel.

There is an urgent need for a rapid genetic test that is scalable without the need for infrastructure that can be used wherever and whenever needed. The present invention addresses these and other needs, and provides related advantages as described herein.

Detection Technologies

Colorimetric sensors using nanoparticles have been used to detect multiple analytes including proteins and nucleic acids to small organic molecules and metal ions. Spherical gold nanoparticles (AuNPs) in solution appear red due to their intense localised surface plasmon resonance at −520 nm. The aggregation of AuNPs induces an electric dipole-dipole interaction and coupling between the plasmons of neighbouring particles, causing the colour to change from pink to blue/purple or clear corresponding with surface plasmon band shifts from 523 nm towards 610˜670 nm (FIG. 1).

Colorimetric sensors have a number of advantages. They are easy to use, typically involving only a single step without requiring trained personnel. They are sensitive with only a few nanoparticles needed to generate visible colour changes due to the extremely high extinction coefficients. Moreover, neither complex nor expensive analytical instruments are needed as the colour change can be detected using the naked eye. Recently, colorimetric assays been employed to detect parts of the N gene of SARS-CoV-2 (ref 6).

However, most of the current detection systems, based on single or two populations of probes have not been readily adapted due to limitations in sensitivity which although sensitive enough for major contaminant detection, are not sensitive enough for pathogen detection. There is a need for improved methods for detecting targeted analytes for pathogens such as SARS-CoV-2. The present invention addresses these and other needs, and provides related advantages as described herein.

Brief Description of the Invention

The present invention relates generally to a nanoparticle-based detection platform for detecting a target nucleic acid in a sample. The detection platform is based on target nucleic acid-dependent agglomeration of nanoparticles that effects a visible colour change. The inventors have surprisingly found that careful selection of oligonucleotide probe sequences that are complementary to target sequences in the target nucleic acid such that the target sequences are spaced-apart or contiguous and non-overlapping, provides sufficient sensitivity and specificity for the detection platform to rapidly and efficiently indicate presence or absence of the target nucleic acid in a sample even in the absence of complex analytical instruments, e.g. by providing a clear colour change of the reaction solution in some cases within hours or minutes and which is visible to the naked eye. This is a welcome improvement over prior described detection platforms, such as that described in WO2005/008222, requiring an immobilised glass slide spot, waveguide and CMOS sensor. Moreover, Moitra et al. 2020 describes a method requiring RNAse treatment (and consequently incubation at elevated temperature) in order to produce a visible colour change.

Accordingly, in a first aspect the present invention provides a method for detecting the presence of a target nucleic acid analyte in a sample, wherein the target nucleic acid analyte comprises at least a first, a second and a third target sequence, which target sequences are spaced-apart or contiguous and non-overlapping, the method comprising: providing a population of oligonucleotide probe-functionalised nanoparticles, said population comprising at least a first oligonucleotide probe that hybridizes to said first target sequence, a second oligonucleotide probe that hybridizes to said second target sequence and a third oligonucleotide probe that hybridizes to said third target sequence; and contacting a solution comprising the sample with the population of nanoparticles, wherein multiple specific binding events between the oligonucleotide probe sequences and target sequences causes agglomeration of the nanoparticles, thereby resulting in a target nucleic acid analyte-dependent visible colour change of the solution. The method of the invention therefore employs detection nanoparticles and target nucleic acid analyte coming into contact in “free-floating” solution rather than immobilised on a substrate as taught in prior art methods. This considerably simplifies production and use of the detection platform since a simple tube (e.g. a single-use tube made from polypropylene for preparing, mixing, centrifuging, transporting and storing solid and liquid samples and reagents) may be employed for mixing the nanoparticles and sample and detection indicated by a colour change visible to the naked eye within minutes or tens of minutes after simple mixing or shaking.

In some embodiments each type of nanoparticle is functionalised with multiple copies of one type of oligonucleotide probe.

In some embodiments the population of nanoparticles comprises a plurality of different types of nanoparticle each functionalised with multiple copies of any of the at least three types of oligonucleotide probe.

In some embodiments the population of nanoparticles is functionalised with at least 4, 5, 6, 7, 8, 9, 10, 11, 12, 13, 14, 15, 16, 17, 18, 19, 20, 21, 22, 23, 24, 25, 30, 35, 40, 45, 50, 55, 60, 65, 70, 75, 80, 95 or at least 100 types of oligonucleotide probe which hybridise to at least 4, 5, 6, 7, 8, 9, 10, 11, 12, 13, 14, 15, 16, 17, 18, 19, 20, 21, 22, 23, 24, 25, 30, 35, 40, 45, 50, 55, 60, 65, 70, 75, 80, 95 or at least 100 corresponding spaced-apart or contiguous non-overlapping target sequences in the target analyte.

In certain embodiments the population of nanoparticles is functionalised with 10 types of oligonucleotide probe which bind to 10 corresponding spaced-apart or contiguous non-overlapping target sequences in the target analyte.

In some embodiments, one or more (e.g. all) of the oligonucleotide probes is perfectly complementary to the target sequence to which it hybridises. However it is specifically contemplated that oligonucleotide probes that hybridise to the target sequence, e.g. despite one or more mismatches (e.g. 1, 2, 3, 4 or 5 base mismatches) may be employed in certain embodiments.

In some embodiments the molar ratio of target to total nanoparticles is selected to permit target-specific nanoparticle agglomeration. In particular, the molar ratio of target to total nanoparticles may be in the range 1:1 to 0.001:1. In certain embodiments the molar ratio may be in the range 0.1 to 0.001. In this context, the ratio is the molar ratio of the concentration of a particular nanoparticle-probe type (“batch”) to the target analyte. This will be different from the cumulative probe concentration. Thus, for example, in an embodiment having 10 types of oligonucleotide probe (10 batches of NPs) the cumulative ratio could be 10:1 probe:analyte, but the molar ratio for each individual NP-probe would be 1:1.

In some embodiments the method comprises a fragmentation step in which said target nucleic acid analyte is broken into two or more fragments. In some cases the fragmentation step may comprise a period of sonication of the sample. For example, sonication may be carried out for at least 10 seconds, at least 30 seconds or at least 60 seconds.

In some embodiments the target analyte comprises viral RNA. In particular, the target analyte may comprise viral genomic RNA, viral sub-genomic mRNA or viral mRNA.

In some embodiments the target analyte comprises SARS-CoV-2 RNA. In some embodiments, the target analyte comprises the genomic sequence of the E protein and/or the N protein of SARS-CoV-2. In some embodiments, the target analyte comprises the sgmRNA of the E gene and/or the N gene of SARS-CoV-2. In some embodiments, the target analyte comprises the leader E-gene and/or the leader N-gene and/or fusion sequence.

In some embodiments the probe sequences are selected from SEQ ID NOs: 1-15. In some embodiments the probe sequences are selected from SEQ ID NOs: 1, 2, 3, 4, and 5. In some embodiments the probe sequences are selected from SEQ ID NOs: 10, 11, 12, 13 and 14. In some embodiments the probe sequences are selected from SEQ ID NOs: 6, 7, 8, 9 and 10. In some embodiments the probe sequences are selected from SEQ ID NOs: 11, 12, 13, 14 and 15. In some embodiments the nanoparticles have a metal core. In particular, the metal core may comprise gold or silver.

In some embodiments the nanoparticles are substantially spherical. In some embodiments, the nanoparticles are non-spherical. In particular, the non-spherical nanoparticle may be multi-branched. For example, the multi-branched nanoparticle may be a nano-urchin. In some embodiments the nanoparticles may be of ellipsoidal or bipyramidal morphology. Mixtures of nanoparticles having differing morphologies are specifically contemplated.

In some embodiments the nanoparticles have a diameter (e.g. mean diameter) between 13 nm and 65 nm, between 20 nm and 60 nm, such as about 30 nm.

In some embodiments the probes comprise DNA or a non-natural nucleic acid. In particular, the probes may comprise locked nucleic acid (LNA), 2′-H nucleic acid, 2′-OMe nucleic acid, 2′-F nucleic acid or peptide nucleic acid (PNA).

In some embodiments the nanoparticle-probe linkage is at the 5′ end of the oligonucleotide probes.

In some embodiments the probes comprise a C6-thiol linkage. In some embodiments the probes comprise a C6-thiol 5′ linkage.

In some embodiments the probe sequences comprise between 10 and 100 nucleotides, between 10 and 50 nucleotides, optionally between 12 and 30 nucleotides. In some embodiments the target sequence hybridising portion of the probe sequences comprises 20 nucleotides.

In some embodiments the probes comprise a nucleotide tail upstream of the target sequence hybridising portion of the probe sequence. In particular, the nucleotide tail may comprise 5 to 20 nucleotides, such as 10 nucleotides. In certain cases the nucleotide tail is a poly-thymine (“poly-T”) tail. In particular, the nucleotide tail may comprise 10 thymines.

In some embodiments the spacing between adjacent nanoparticles when the probes are hybridised to the target sequences of the target analyte is between 5 nm and 30 nm.

In some embodiments the spacing between adjacent nanoparticles when the probes are hybridised to the target sequences of the target analyte is between 10 nm and 18 nm.

In some embodiments the spacing between adjacent nanoparticles when the probes are hybridised to the target sequences of the target analyte is 12 nm.

In some embodiments the method further comprises a step of adding extra salt.

In some embodiments, the method further comprises a step of adding Sodium Dodecyl Sulfate (SDS) and proteinase K. In some embodiments, the final concentration of SDS is at least 0.5%.

In some embodiments, the method is carried out at less than 45° C. For example, the method may be carried out at less than 40° C., less than 35° C., less than 25° C., or less than 20° C.

In some embodiments, the method does not involve addition of RNAse. For example, RNAse is not added after NP agglomeration.

In a second aspect the present invention provides a population of oligonucleotide probe-functionalised nanoparticles comprising at least a first, a second and a third oligonucleotide probe which hybridise to at least a first, a second and a third spaced-apart or contiguous and non-overlapping target sequence in a target nucleic acid analyte.

In some embodiments the population of oligonucleotide probe-functionalised nanoparticles is for use in a method of the first aspect of the invention.

The population of oligonucleotide probe-functionalised nanoparticles may be as defined in accordance with the first aspect of the invention.

In some embodiments each type of nanoparticle is functionalised with multiple copies of one type of oligonucleotide probe.

In some embodiments the population of nanoparticles comprises a plurality of different types of nanoparticle each functionalised with multiple copies of any of the at least three types of oligonucleotide probe.

In some embodiments the population of nanoparticles is functionalised with at least 4, 5, 6, 7, 8, 9, 10, 11, 12, 13, 14, 15, 16, 17, 18, 19, 20, 21, 22, 23, 24, 25, 30, 35, 40, 45, 50, 55, 60, 65, 70, 75, 80, 95 or at least 100 types of oligonucleotide probe which hybridise to at least 4, 5, 6, 7, 8, 9, 10, 11, 12, 13, 14, 15, 16, 17, 18, 19, 20, 21, 22, 23, 24, 25, 30, 35, 40, 45, 50, 55, 60, 65, 70, 75, 80, 95 or at least 100 corresponding spaced-apart or contiguous non-overlapping target sequences in the target analyte.

In certain embodiments the population of nanoparticles is functionalised with 10 types of oligonucleotide probe which bind to 10 corresponding spaced-apart or contiguous non-overlapping target sequences in the target analyte.

In some embodiments, one or more (e.g. all) of the oligonucleotide probes is perfectly complementary to the target sequence to which it hybridises. However it is specifically contemplated that oligonucleotide probes that hybridise to the target sequence, e.g. despite a single base mismatch may be employed in certain embodiments.

In some embodiments the target analyte comprises viral RNA. In particular, the target analyte may comprise viral genomic RNA, viral sub-genomic mRNA or viral mRNA.

In some embodiments the target analyte comprises SARS-CoV-2 RNA. In some embodiments, the target analyte comprises the genomic sequence of the E protein of SARS-CoV-2. In some embodiments, the target analyte comprises the sgmRNA of the E gene of SARS-CoV-2.

In some embodiments the probe sequences are selected from SEQ ID NOs: 1, 2, 3, 4, 5 and 6. In some embodiments the probe sequences are selected from SEQ ID NOs: 10, 11, 12, 13 and 14. In some embodiments the probe sequences are selected from SEQ ID NOs: 7, 8 and 9.

In some embodiments the nanoparticles have a core comprising metal or diamond. In particular, the core may comprise gold.

In some embodiments the nanoparticles are substantially spherical.

In some embodiments the nanoparticles have a diameter (e.g. mean diameter) between 13 nm and 65 nm, between 40 nm and 60 nm, such as about 50 nm.

In some embodiments the probes comprise DNA or a non-natural nucleic acid. In particular, the probes may comprise locked nucleic acid (LNA), 2′-H nucleic acid, 2′-OMe nucleic acid, or 2′-F nucleic acid.

In some embodiments the nanoparticle-probe linkage is at the 5′ end of the oligonucleotide probes.

In some embodiments the probes comprise a C6-thiol linkage. In some embodiments the probes comprise a C6-thiol 5′ linkage.

In some embodiments the probe sequences comprise between 10 and 100 nucleotides, between 10 and 50 nucleotides, optionally between 12 and 30 nucleotides. In some embodiments the target sequence hybridising portion of the probe sequences comprises 20 nucleotides.

In some embodiments the probes comprise a nucleotide tail upstream of the target sequence hybridising portion of the probe sequence. In particular, the nucleotide tail may comprises 5 to 20 nucleotides, such as 10 nucleotides. In certain cases the nucleotide tail is a poly-thymine (“poly-T”) tail. In particular, the nucleotide tail may comprises 10 thymines.

In some embodiments the spacing between adjacent nanoparticles when the probes are hybridised to the target sequences of the target analyte is between 5 nm and 30 nm.

In some embodiments the spacing between adjacent nanoparticles when the probes are hybridised to the target sequences of the target analyte is between 10 nm and 18 nm.

In some embodiments the spacing between adjacent nanoparticles when the probes are hybridised to the target sequences of the target analyte is 12 nm.

In a third aspect the present invention provides a kit for detection of a target nucleic acid analyte in a sample, the kit comprising: a population of nanoparticles of the second aspect of the invention; a reaction vessel for holding a solution, the reaction vessel comprising at least a wall and a sealable opening, wherein visible light is able to pass through at least a portion of the wall; and one or more reagents or solutions for carrying out the method of the first aspect of the invention.

In some embodiments the kit further comprises one or more reagents or solutions for isolating target nucleic acid from a sample. In some embodiments the solution may be for isolating and/or purifying the target nucleic acid from a sample. One such exemplary solution is as provided in the Coronavirus RNA extraction research kit available from ARCIS Biotechnology (Daresbury, UK). In some cases the kit may comprise “Reagent 1” and/or “Reagent 2a” of said Coronavirus RNA extraction research kit as described in the protocol dated 28 Mar. 2020 and available at the following URL: https://arcisbio.com/wp-content/uploads/2020/03/SARS-CoV-2-Protocols-V3-20200328-1.pdf. In some embodiments the Kit further comprises a colour reference (e.g. a colour card) corresponding to the expected colour change upon positive detection of the target nucleic acid analyte. This may be used to facilitate recognition of the colour change by the user, e.g. by holding the reaction vessel up to the colour reference.

In a fourth aspect the present invention provides a device for detecting the presence of a target nucleic acid analyte in a sample, the device comprising: an inlet for receiving a sample, a passage for the sample to pass from the inlet to a storage compartment pre-loaded with a set of reagents, wherein the reagents comprise the population of nanoparticles according of the second aspect of the invention, and a detection window comprising a colour reference, wherein, in use, the sample contacts the reagents in the storage compartment, so that the positive detection of a target nucleic acid analyte results in an expected colour change in the colour reference visible through the detection window.

In some embodiments, the device further comprises a cap. In particular, the cap may comprise NaCl such that, upon closing the cap, the NaCl is added to the storage compartment.

In some embodiments the device may be used in a method according to the first aspect of the invention.

### BRIEF DESCRIPTION OF THE FIGURES

FIG. 1—A. Schematic diagram of signal amplification concept. B. Schematic diagram showing use of a mixture of distinct populations of NP-probes with each NP having multiple copies of the same probe. C. Schematic diagram showing use of multiple copies of different probes on the same NP. D. Schematic diagram of signal amplification.

FIG. 2—Schematic diagram of typical β-coronavirus. The replicase gene of is comprised of ORFs 1a and 1b, which are located distal to the 5′ UTR and the leader sequence found at the 5 end of the genome. The structural protein genes S, E, M and N, are located proximal to the 3′ UTR. Interspersed between the structural protein genes are the accessory genes encoding non-structural proteins, which are not essential for replication in vitro. Reproduced from Armesto et al.

The replication cycle of a coronavirus following infection of a susceptible cell. Genomic RNA is released and acts as a mRNA for the translation of the replicase proteins. Nested sub-genomic mRNAs (sgmRNAs) are produced from the genomic RNA for the expression of the structural and accessory proteins. Figure not shown, but available from Armesto et al.

FIG. 3—Colorimetric change in response to specific analyte. A. Demonstration of colour change of nanoparticle probes in presence of matching analyte and mismatch analyte that differs by one nucleotide over time. B. Spectra shift over time. C. Decrease in free nucleic acid analyte over time as measured by absorbance at 260 nm.

FIG. 4—Specificity of binding using DNA analyte. Binding kinetics as assessed by surface plasmon resonance of two adjacent 20-mer oligonucleotide probes to either a wild-type BRAF gene sequences or mutant sequence that differs by 1/100 nucleotides.

FIG. 5—Change in sensitivity of detection of specific analyte at differing concentrations according to size of nanoparticle. (Top) 63 nm (Middle) 46 nm (Bottom) 13 nm nanoparticle.

FIG. 6—Comparison of 25 nm and 50 nm nanoparticles to detect either 70 nt or 140 nt ssDNA or dsDNA analytes using different probe chemistries (unmodified DNA, 2′-OMe and 2′-F).

FIG. 7—Comparison of agglomeration stability over time. A. Under irradiation at 37° C. B. Without irradiation at 37° C. C. Without irradiation at room temperature.

FIG. 8—Comparison of detection sensitivity using single nucleotide differences between target analytes for the BRCA1 and EGFR genes. A. Match. B. Mismatch. C. Match and mismatch.

FIG. 9—A. UV-Vis spectra for the different combination of NPs (150 uM) for different concentration of 108 nt target sequence. B. The change of absorbance at the maximum of surface plasmon band and the change of R value (R=Abs70/Abs530) with varying analyte concentration for assay containing different number of batches.

FIG. 10—A. UV-Vis spectra for the different combination of NPs (100 uM) for different concentration of 108 nt target sequence. B. The change of absorbance at the maximum of surface plasmon band and the change of R value (R=Abs70/Abs530) with varying analyte concentration for assay containing different number of batches.

FIG. 11—A-E. UV-vis-NIR spectra for the different combination of NPs (1, 2, 3, 4 and 5) at a concentration of 150 μM for different concentration of 108 nt target sequence. F. Image of the different assays changing the number of NPs and the concentration of the target. G. The change of R value (R=Abs700/Abs530) with varying analyte concentration for assay containing different number of batches. H. The change of absorbance at the maximum of surface plasmon band, 530 nm. I. The shift in the position of the maximum of surface plasmon band. Limit of detection of the assay is estimated to be 1 nM.

FIG. 12—A-D. UV-vis-NIR spectra for the different combination of NPs (2, 3, 4 and 5) at a concentration of 100 μM for different concentration of 108 nt target sequence. E. Image of the different assays changing the number of NPs and the concentration of the target. F. The change of R value (R=Abs700/Abs530) with varying analyte concentration for assay containing different number of batches. G. The change of absorbance at the maximum of surface plasmon band, 530 nm. H. The shift in the position of the maximum of surface plasmon band. Limit of detection of the assay is estimated to be 100 μM for assay of 4 and 5 batches.

FIG. 13—A-D. UV-vis-NIR spectra for the different combination of NPs (2, 3, 4 and 5, respectively) at a concentration of 50 μM for different concentration of 108 nt target sequence. E. The change of R value (R=Abs700/Abs530) with varying analyte concentration for assay containing different number of batches. F. The change of absorbance at the maximum of surface plasmon band, 530 nm. G. The shift in the position of the maximum of surface plasmon band.

FIG. 14—A. UV-Vis spectra of 2, 3, 4, and 5 NP batches (E1-E5) at sonication period of 30, 60, and 150 sec. B. UV-Vis of 2, 3, 4, 5 NP batches (E12-E16) at sonication time of 30, 60, and 150 sec. C. Aggregation degree, R, (upper panel) and Abs@530 nm (lower panel) for different number of NPs batches and sonication time.

FIG. 15—A. UV-vis-NIR spectra of 2, 3, 4, and 5 NP batches (E1-E5). B. UV-Vis of 2, 3, 4, 5 NP batches (E12-E16). C. Image of the different assays changing the number of NPs and capture probes sets. D. Aggregation degree (R) for different number of NPs batches. E. Abs@530 nm for different number of NPs batches. F. Plasmon band position for different number of NPs batches.

FIG. 16—A. UV-Vis spectra (upper panel) and aggregation degree (lower panel) of 2 NP batches. B. UV-Vis spectra (upper panel) and aggregation degree (lower panel) of 3 4 and 5 NP batches.

FIG. 17—A. Melting curve calculations for different probe sequences according to 1, 2 or 3 nt changes in the sequence of the analyte and according to position for E1-5. B. Melting curve calculations for different probe sequences according to 1, 2 or 3 nt changes in the sequence of the analyte and according to position for E12-16.

FIG. 18—Detection of a fragment of synthetic subgenomic E gene analyte using 50 nm nano-urchins. A. Image of the colour change two minutes after adding the target analyte, the 3′ half of the E Gene (120 nt-long sequence of subgenomic RNA). The control wells contain a 108 nt-long RNA sequence that is non-complementary to the AuNP probes. B. Corresponding UV-vis spectra for the target RNA sequences and controls. Experiments were carried out in triplicate.

FIG. 19—Detection of a fragment of synthetic subgenomic E gene analyte using spherical 30 nm AuNPs. A. Image of the colour change two minutes after adding the target analyte, the 3′ half of the E Gene (120 nt-long sequence of subgenomic RNA). The control wells contain a 108 nt-long RNA sequence that is non-complementary to the AuNP probes. B. Corresponding UV-vis spectra for the target RNA sequences and controls. Experiments were carried out in triplicate.

FIG. 20—Detection of a synthetic N gene RNA with 30 nm nanospheres. UV-vis spectra for gold nanospheres functionalised with a mix of seven probes (probes N1-7) or six probes (N8-20) detecting a range of different concentrations (from 100 nM to 0.1 nM) of a synthetic N gene RNA fragment (1260 nt). Experiments were carried out in triplicate. W=water only control.

FIG. 21—Detection of SARS-CoV-2 full viral sequence using 30 nm AuNPs functionalised with a mix of N gene probes (N1-7 and N8-20). A. Image of the colour change adding 10 fmol or 1 fmol of target analyte (SARS-CoV-2 full viral sequence). B. Corresponding UV-Vis spectra for the different concentrations of target analyte (SARS-CoV-2 full viral sequence). Experiments were carried out in duplicate. W=water only control.

FIG. 22—Detection of SARS-CoV-2 full viral sequence using 30 nm AuNPs functionalised with a mix of E gene probes (E7-17). A. Image of the colour change adding 10 fmol or 1 fmol of target analyte (SARS-CoV-2 full viral sequence). B. Corresponding UV-Vis spectra for the different concentrations of target analyte (SARS-CoV-2 full viral sequence). Experiments were carried out in duplicate. W=water only control.

FIG. 23—Detection of SARS-CoV-2 full viral sequence using 30 nm AuNPs functionalised with a mix of N and E gene probes. A. Image of the colour change adding 10 fmol or 1 fmol of target analyte (SARS-CoV-2 full viral sequence). B. Corresponding UV-Vis spectra for the different concentrations of target analyte (SARS-CoV-2 full viral sequence). Experiments were carried out in duplicate. W=water only control.

FIG. 24—Specificity of detection of SARS-CoV-2. A. Image of the colour change when using genomic RNA from feline alphacoronavirus VR-989 as target analyte vs. when using genomic RNA from SARS-CoV-2 as target analyte. B. Corresponding UV-Vis spectra for the different target analytes. Experiments were carried out in triplicate.

FIG. 25—Addition of salt after nanoparticle addition enhances detection. UV-Vis spectra for AuNPs functionalised with different probes: E7-17 (A); E1-6 (B); N1-7 and N8-20 (C); N13 (D), and after addition of 0.7M extra salt, with a final salt concentration of 1.5M. Experiments were carried out in duplicate. Water was used as a control.

FIG. 26—Determining the limit of detection (LOD). A. Image of the colour change for AuNPs functionalised with probes E7-17 and assayed with a range of known concentrations of full-length genomic SARS-CoV-2 RNA. B. Corresponding UV-Vis spectra for the different concentrations of RNA.

FIG. 27—Determining the LOD. A. Image of the colour change for AuNPs functionalised with E gene (11 probes) and leader sequence (17 probes) probes and assayed with a range of known concentrations of full-length genomic SARS-CoV-2 RNA. B. Corresponding UV-Vis spectra for the different concentrations of RNA.

FIG. 28—Detection of SARS-CoV-2 RNA in saliva. A. UV-Vis spectra for AuNPs functionalised with E7-E17 probes assayed with SARS-CoV-2 RNA spiked-in saliva. B. UV-Vis spectra for AuNPs functionalised with E7-E17 probes assayed with SARS-CoV-2 in saliva and using SDS and proteinase K. Experiments were carried out in duplicate.

FIG. 29—Detection of SARS-CoV-2 RNA in saliva using patient-derived samples. A. Image of the colour-change for AuNPs functionalised with E7-17 probes assayed with SARS-CoV-2-positive patient-derived samples, namely saliva with spiked-in RNA from nasopharyngeal samples. B. UV-Vis spectra for AuNPs functionalised with E7-E17 probes assayed with SARS-CoV-2-positive patient-derived samples, and using SDS and proteinase K.

FIG. 30—Clinical testing of individual samples. A. Example of samples tested in a blinded fashion by six independent observers marking tests as positive or negative by eye. B. Table containing clinical tests parameters calculated based on the aggregated results from A.

FIG. 31—Prototype of an all-in-one COVID-19 molecular test

Table 1—RNA analyte detection with 23 nt RNA analyte and 50 nm

NPs

Table 2—RNA analyte detection with 70 nt RNA analyte and 25 nm NPs

Table 3—Oligonucleotide target sequences and capture probes

Table 4—Oligonucleotide sequences of the capture probes

Table 5—Ratio of target molecules and number of gold nanoparticles for different concentration of Auₒ and different concentrations of target used in the assays

Table 6—Melting curve calculations for different probe sequences according to 1, 2 or 3 nt changes in the sequence of the analyte and according to position of change

### DETAILED DESCRIPTION OF THE INVENTION

The present invention is based on the principle of using multiple (e.g. 3 or more) contiguous non-overlapping targeting probe sequences attached to nanoparticles (NPs) that specifically bind nucleic acid sequences of pathogens, such as viral or bacterial pathogens. Specific multiple binding events bring NPs in close proximity to each other effectively causing agglomeration of NPs. The agglomeration leads to a visual colour change only in the presence of the analyte that can be detected by the naked eye. Alternatively, for example, when the NP comprises diamond, the agglomeration leads to a change in luminescence, detectable optically and convertible to a visual change that can be detected by the naked eye. A mixture of multiple probes targeting different regions of the RNA or DNA analyte results in inter-molecular amplification of the signal, increasing the sensitivity of the detection system.

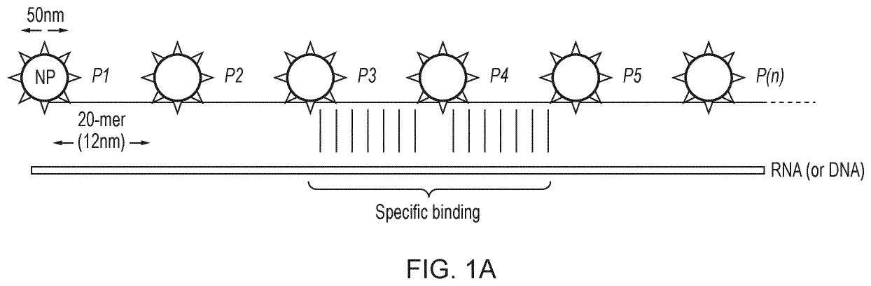

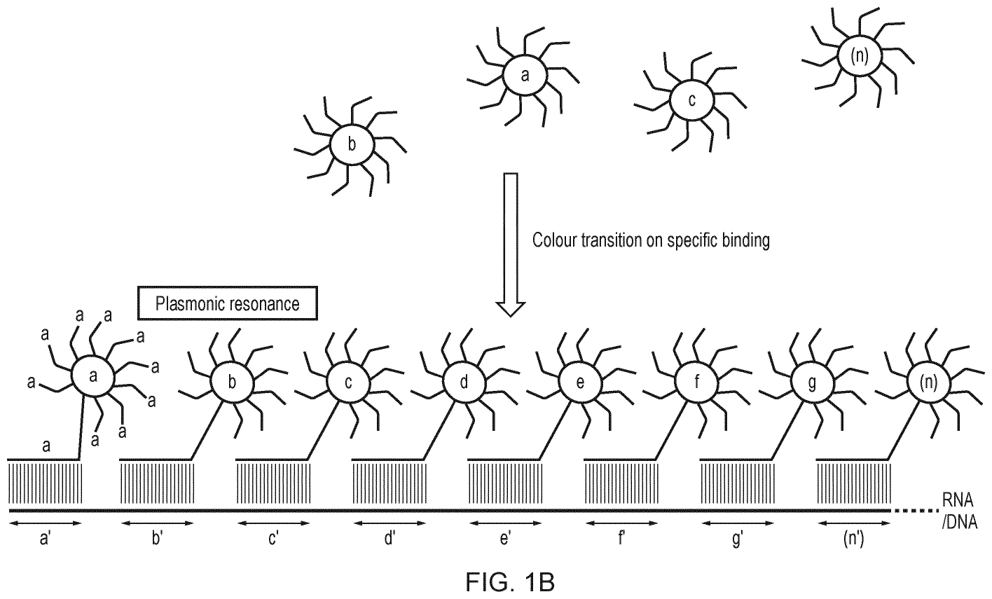

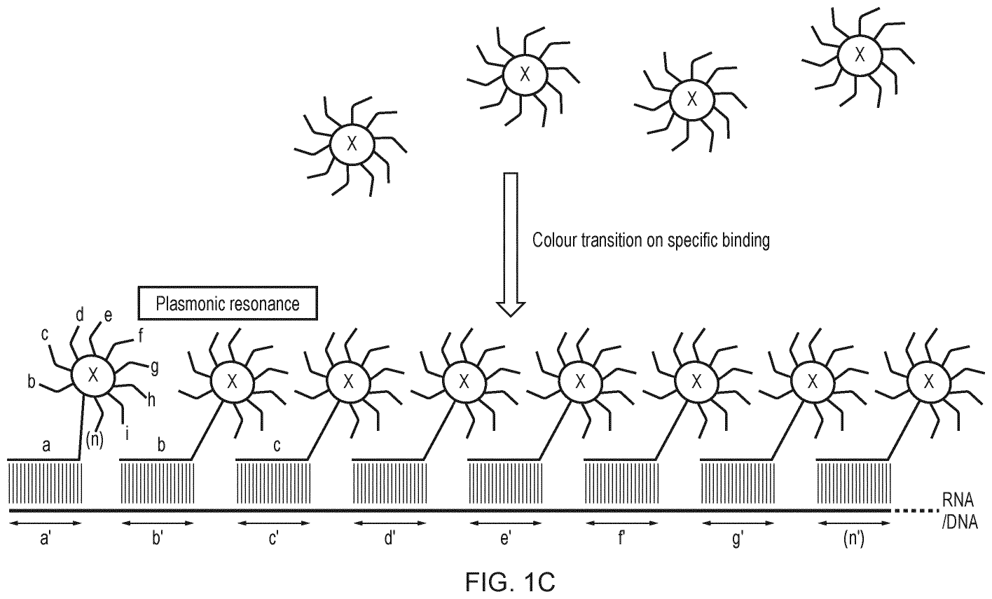

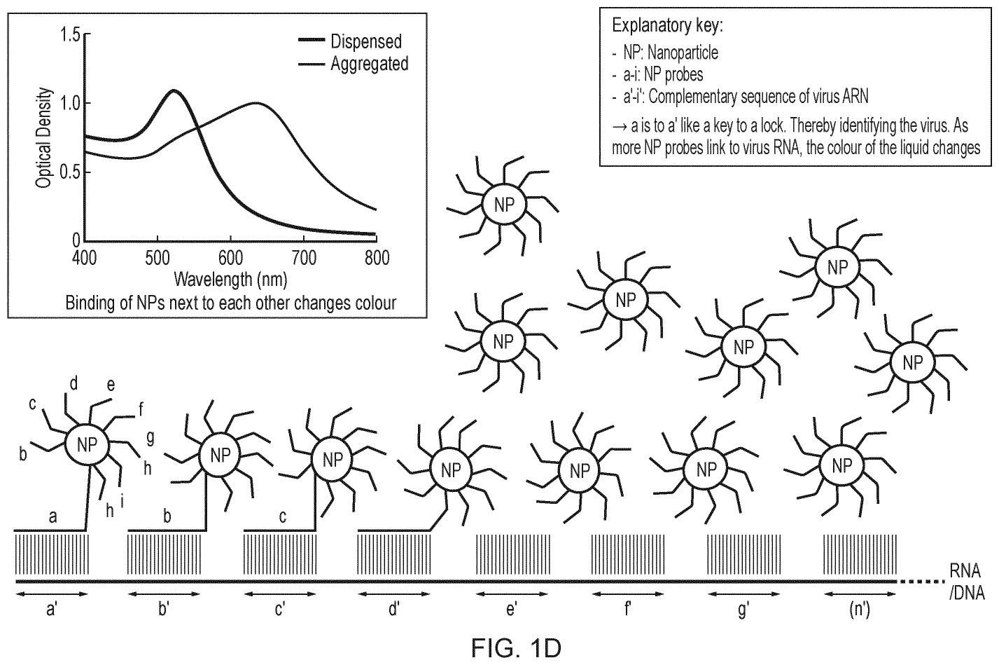

Advantageously, the sensitivity and signal amplification of analyte binding resulting in visual colour change is enhanced by the use of multiple probes for a specific long RNA/DNA sequence. The number of probes could be between 3 and n. The addition of more probes leads to an additive increase in sensitivity of the detection system (FIG. 1A).

The present invention may use a mixture of distinct populations of NP-probes with each NP having multiple copies of the same probe (FIG. 1B). Alternatively, the present invention may use multiple copies of different probes on the same NP (FIG. 1C). The NPs as shown in FIG. 1C may be made by using a mix of DNA probes rather than single sequences when carrying out the functionalisation of the NPs. Without wishing to be bound by theory, the inventors believe that this creates a random distribution of the probes on the NPs and, as NP-probes with the highest affinity would be selected to bind preferentially over lower affinity NP-probes, providing higher sensitivity. Different populations of NP-probes may be described in certain places herein as “batches”.

The present invention provides a platform technology. The present invention finds use for multiple analytes. In preferred embodiments, the present invention may be for use in the detection of nucleic acids from pathogens such as viruses or bacteria in humans, animals, food or the environment. The present invention can be used for rapid, simple-to-use and economic detection of SARS-CoV-2 virus from clinically obtained samples.

SARS-CoV-2

The Coronaviridae form part of the order Nidovirales, which comprises two sub-families, the Coronavirinae and Torovirinae. The Coronavirinae family of viruses are named for their visual resemblance to the corona of the sun in negatively stained preparations. There are three genera of coronaviruses, alpha-, beta—and gamma-coronaviruses. The SARS-CoV-2 virus is a beta-coronavirus which is closely related to Severe acute respiratory syndrome-associated virus (SARS).

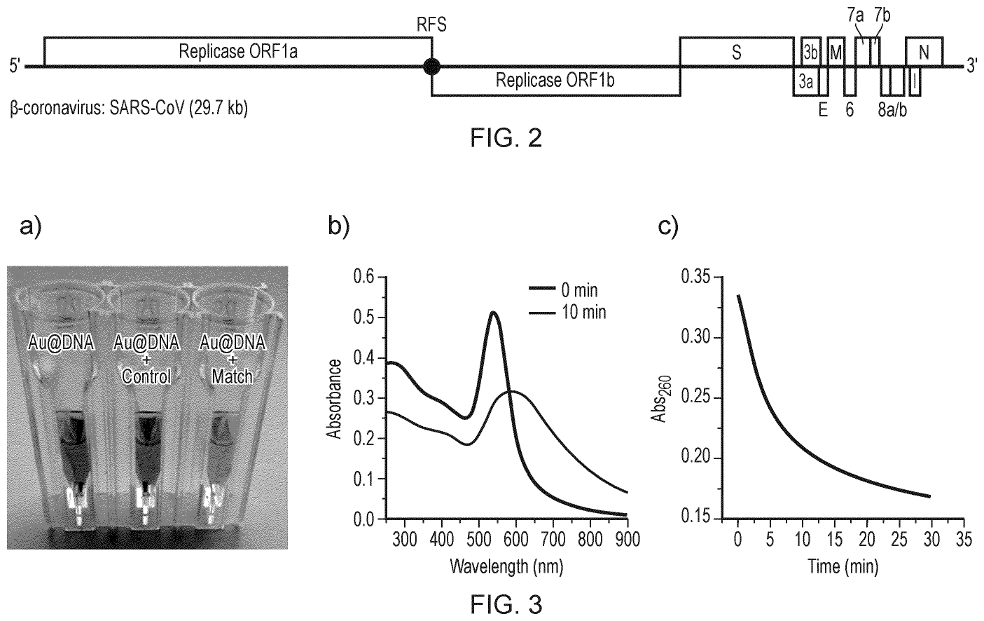

Coronaviruses are enveloped viruses with a single-stranded positive-sense RNA genome of 26-32 kb, and represent the largest genomes of any RNA virus, with the SARS-CoV-2 virus having a genome of 29,903 nucleotides (RefSeq NC 045512). The genome associates with the nucleoprotein (N), forming a helical nucleocapsid within the virus particles. These are enclosed within lipid envelopes containing the spike (S) glycoprotein, membrane (M) protein and envelope (E) protein. The genomic organisation of beta-coronaviruses is shown in FIG. 2.

Although coronaviruses contain a positive-strand RNA genome that can directly act as template for protein translation, during the infective cycle they also produce negative-strand copies of the genome both in its entirety during continuous transcription, and a subset of non-continuous sub-genomic (sg) negative-strand mRNAs.

These sgmRNAs are produced by the presence of a transcription regulation sequence (TRS) that either cause a pausing of transcription process or termination of the process leading to the generation of nested sgmRNA fragments. As transcription of the sgmRNAs initiates at the 3″-end of the coronavirus genome (5″ of negative strand) there is probabilistically higher quantities of 3′ genes including the E, M and N proteins, than of 5′ genes such as ORF1 and S. Indeed, compared to the positive strand genomic RNA genome, there are significantly more sgmRNA transcripts in infected cells with reports of x70-fold increase (Hofmann et al J Virol 64; 4108-4114). It is expressly contemplated herein that probes of the NPs of the invention may target the genomic positive strand sequence and/or non-overlapping mRNA or sgmRNA sequences.

Detection Kits

The present invention provides a detection kit, whereby purified analyte is added to a solution. Solution 1 is a third party purification solution. In certain embodiments, solution 1 may be as provided in the Coronavirus RNA extraction research kit available from ARCIS Biotechnology (Daresbury, UK). In some cases Solution 1 may comprise two solutions which are “Reagent 1” and “Reagent 2a” of said Coronavirus RNA extraction research kit as described in the protocol dated 28 Mar. 2020 and available at the following URL: https://arcisbio.com/wp-content/uploads/2020/03/SARS-CoV-2-Protocols-V3-20200328-1.pdf. “Solution 2” comprises the nanoparticles of the invention and a solvent/buffer for detection. In another embodiment, the present invention may be in the form of a lateral flow assay through the immobilisation of the NP-probes to a detection surface.

SDS and Proteinase K

The use of the SalivaDirect™ method, i.e. adding SDS/Proteinase K avoids the need for a separate RNA extraction step and allows for a one-step detection method of the target analyte (https://publichealth.yale.edu/salivadirect/). Unlike PCR, which requires the removal of both SDS and proteinase K before testing, SDS is a well-known stabiliser of NPs. PCR relies on the presence of enzymes, which requires the removal of proteinase K. Advantageously, there are no proteins in the present system, so removal of proteinase K is not necessary. Another advantage of using the SDS/Proteinase K method is that the SDS inactivates the virus, and a concentration of 0.5% SDS is sufficient to completely destroy the virus.

Other enzymes possibly mixed with other serine endopeptidases such as subtilisin A and metalloproteases could be used as alternatives to proteinase K.

Nanoparticles

Nanoparticles used in a detection kit may preferably comprise gold but may also be silver or any other metal. NPs may also comprise diamond. The NPs may be hollow or solid. The NPs may preferably be spherical but may also be non-spherical such as stars, rods or other shapes. The non-spherical NPs may be nano-urchins (Sigma). The diameter of NPs may in some cases have a diameter in the range 5 nm to 200 nm, preferably 20 nm to 100 nm, more preferably 30 nm to 70 nm, such as around 50 nm in diameter. Without wishing to be bound by theory, the present inventors believe that smaller nanoparticles such as those having a mean diameter of 25 nm may be more stable in solution and that larger nanoparticles such as those having a mean diameter of around 65 nm may increase the sensitivity of the assay. NPs may be made using the Turkevich method, seeded growth method, or other suitable methods which are well known to the skilled person. The use of nanodiamonds is expressly contemplated herein. In particular, the use of fluorescent nanodiamonds is contemplated herein (ref 8). Nanodiamonds are carbon-based nanoparticles with a core of diamond carbon and a surface shell that is partially graphite-based (ref 9). Their size ranges from 1 to 100 nm. Modifications of diamond film surfaces with biomolecules such as DNA have been described (ref 10).

Probes

Probes used in a detection kit could be made from DNA or modified nucleic acids such as PNA, LNA, 2′-H, 2′-OMe, 2′-F. The probe may have a covalent attachment to the nanoparticle surface, for example via a thiol linker. In some cases, the probe may be attached via a C6-thiol 5′-linkage, but could be 3′ or internal. The probes are attached via thiol linkage and could contain PEG or similar spacer molecules to minimise non-specific agglomeration. In certain cases the probes may be linked to the nanoparticle via a spacer of chain length 10 to 50 atoms. For example, a C18 spacer. The spacer may be employed in addition to the thiol linker. For example the probes may be linked to the nanoparticle surface via a C6-thiol linker and a C18 spacer.

The probe sequences are preferably 20-mer although they can vary between 12-30 nucleotides in length. The probe sequences can hybridise to the target sequences under medium or high stringency conditions. In some cases the salt concentration may be 100 μM to 500 μM, e.g. 100 μM to 500 μM NaCl. In certain embodiments the salt concentration may be 150 μM NaCl. In certain embodiments the temperature for allowing hybridisation may be in the range 25-30° C. Preferably, the probe sequences are complementary to the target sequences. However, as shown herein, hybridisation may be sufficiently strong to enable detection of target analyte even when the target-probe sequences exhibit one more more base mismatches, such as 1, 2 or 3 mismatches. The probe sequences are screened for non-specific targets, self-complementarity, Tm value and evolutionary conservation of the target. They can, for example in the case of Coronavirus or similar, target the genomic sequence, sub-genomic fragments or mRNAs of the pathogen.

The target nucleic acid analyte comprises at least a first, a second and a third non-overlapping target sequence, wherein the target sequences are different and may be spaced-apart or contiguous. Accordingly, the at least first, second and third oligonucleotide probe sequences of the invention are different.

Nanoparticle-probes may be referred to as NP@DNA. Nanoparticle-probes wherein the nanoparticle has a gold core may be referred to as Au@DNA.

Spacing Between Nanoparticle Probes

The spacing between adjacent nanoparticles when bound to the target analyte may be between 5 nm and 30 nm. The spacing between adjacent nanoparticles when bound to the target analyte may be between 13 nm and 18 nm. The spacing between adjacent nanoparticles may be measured by electron microscopy. Alternatively or additionally, the spacing between adjacent nanoparticles may be determined theoretically based on the lengths of the target sequences and distances between the target sequences. The average length of a nucleotide is 0.6 nm, so with a 20-mer target sequence the theoretical distance between NPs would be 12 nm. This distance can be adjusted, for example by varying the probe sequence either in position of binding or physically extending the probe length.

Samples

Samples may include any biological liquid or tissue sample and/or environmental sample. In particular embodiments, the sample may be saliva or a nasopharyngeal sample. The samples may be from SARS-CoV-2 infected individuals. Samples may be RNA from a SARS-CoV2 infected individuals spiked in into saliva samples. Samples may also be obtained from a cell culture. For example, samples may be obtained from a cell culture established using cells infected with SARS-CoV-2.

### EXAMPLES

### Example 1—Synthesis and Functionalization of Gold Nanoparticles

Gold nanoparticles (AuNPs) were synthesized following the seeded growth method. The seeded growth process comprises the cyclic addition of metal precursor and extraction of particles. In a typical process, the seed solution is cooled down to 90° C. and then HAuCl₄ solution (25 mM) is added, followed by a second addition after 30 min. After a further 30 min period, the growth solution is extracted and sodium citrate solution (60 mM) added. This process is repeated to increase the size of the resulting AuNPs.

AuNPs are functionalized with thiolated oligonucleotide primers (see Example 2 for exemplary oligonucleotide probe sequences “primers”) according to the method of Hurst et al. In brief, colloidal AuNPs in SDS (0.1%) and PB (0.01 M) are added to a 3.6 μM solution of the oligonucleotides. The mixture of oligonucleotides and AuNPs are incubated at room temperature for 20 min, and to improve the oligonucleotide binding, a salt aging process is carried out. A solution containing NaCl (2 M), SDS (0.01%), and PB (0.01M) is added sequentially to the mixture to reach a final NaCl concentration of 0.2 M. Each salt aging step is alternated with sonication (10 s) and incubation (20 min), followed by incubation for 12 h. To remove excess oligonucleotides, the solutions are centrifuged 3 times and each time re-dispersed in SDS (1 mL, 0.01%).

Citrate-stabilized AuNPs were functionalized with single stranded DNA (ssDNA) according to recent protocols (Mirkin et al., Anal Chem, 2006, Vol. 7, pp 8313-8318—incorporated herein by reference). AuNPs stabilized with DNA exhibit colloidal stability in an aqueous solution containing anionic surfactant-sodium dodecyl sulphate (SDS), 0.01 wt %, as confirmed by UV-Vis spectroscopy. The plasmon band redshifts−3 run in all samples after DNA binding, suggesting the formation of a molecular shell around the nanoparticle's surface. TEM analysis confirms the formation of a molecular shell of a thickness of−1.4 nm.

### Example 2—Colorimetric Change in Response to Specific Analyte

The agglomeration process leads to a change in colour of colloidal nanoparticles containing specific probes upon binding to the analyte (FIG. 3). In some cases the colour change may take place over a period of several hours or tens or minutes. In certain optimised cases the colour change may take place in 10-20 mins. However, longer time periods are also contemplated.

### Example 3—Specificity of Binding Using DNA Analyte

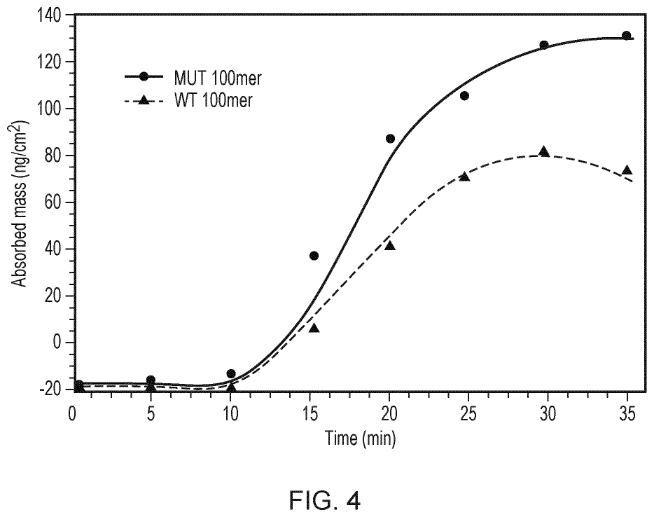

The inventors have demonstrated that a nanoparticle-based probe system can be used to distinguish between DNA analytes even when they only vary in a single nucleotide. FIG. 4 shows preferential binding of the designed mutant BRAF probe to the mutant BRAF gene sequence as compared with the wild-type BRAF sequence. This demonstrates that the designed probes can bind specifically to mutated sequences of interest.

### Example 4—Sensitivity According to NP Size

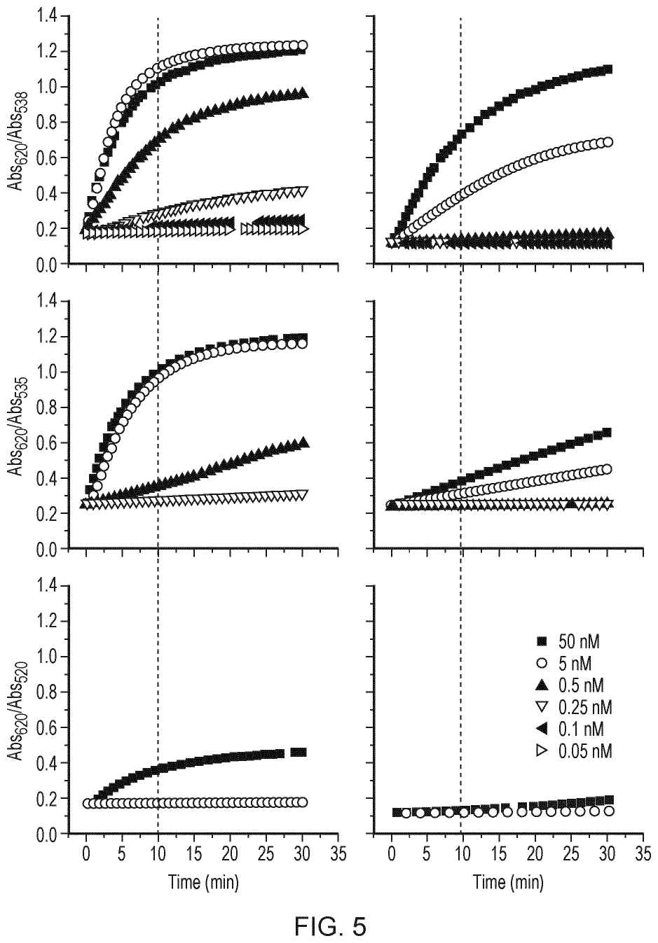

The specificity of detection increases with the size of the nanoparticle used in the system (FIG. 5).

### Example 5—Sensitivity According to Analyte Size

Comparison of analyte target length suggests that longer target lengths are more efficient and have more rapid binding kinetics than shorter lengths (Data not shown).

### Example 6—Comparison of the Ability of Different Nucleic Acid Probe Chemistries to Detect ssDNA and dsDNA Analytes

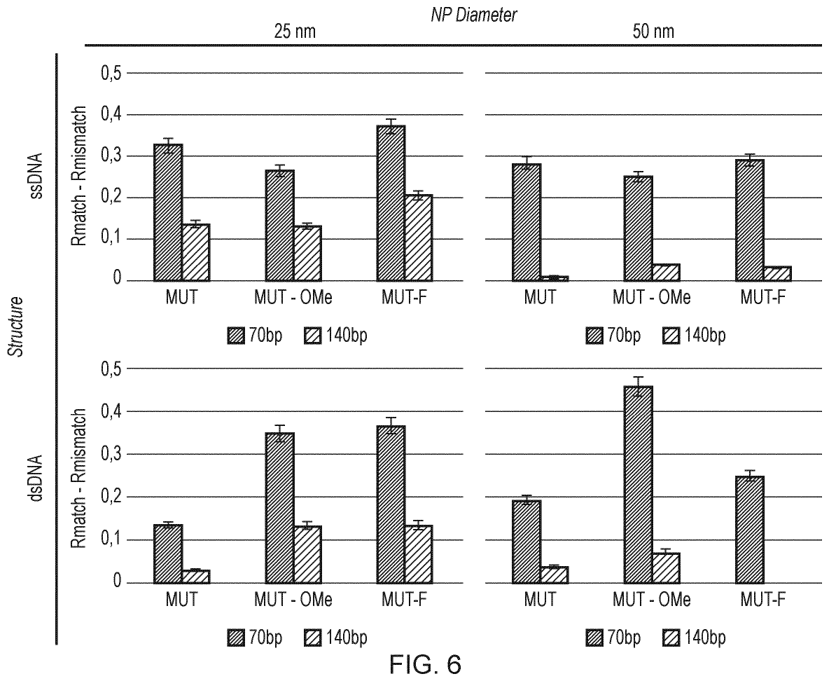

Three different nucleic acid chemistries (i.e. unmodified DNA, 2′-OMe and 2′-F) attached to either 25 nm or 50 nm diameter nanoparticles were compared for their ability to detect either ssDNA or dsDNA analytes (FIG. 6).

The 2′-F modification improved the sensitivity of ssDNA detection, especially for the smaller NPs. Without wishing to be bound by theory, the present inventors theorise that 2′-OMe modification is better for detection of dsDNA while 2′-F modification performs better for ssDNA.

The functionalization of gold nanoparticles with peptide nucleic acid (PNA) was also tried, but it was not possible to stabilize the particles in solution with this modified-oligonucleotides due to the lack of charge along the peptide chain.

### Example 7—Stability of NP Agglomerates with Temperatures

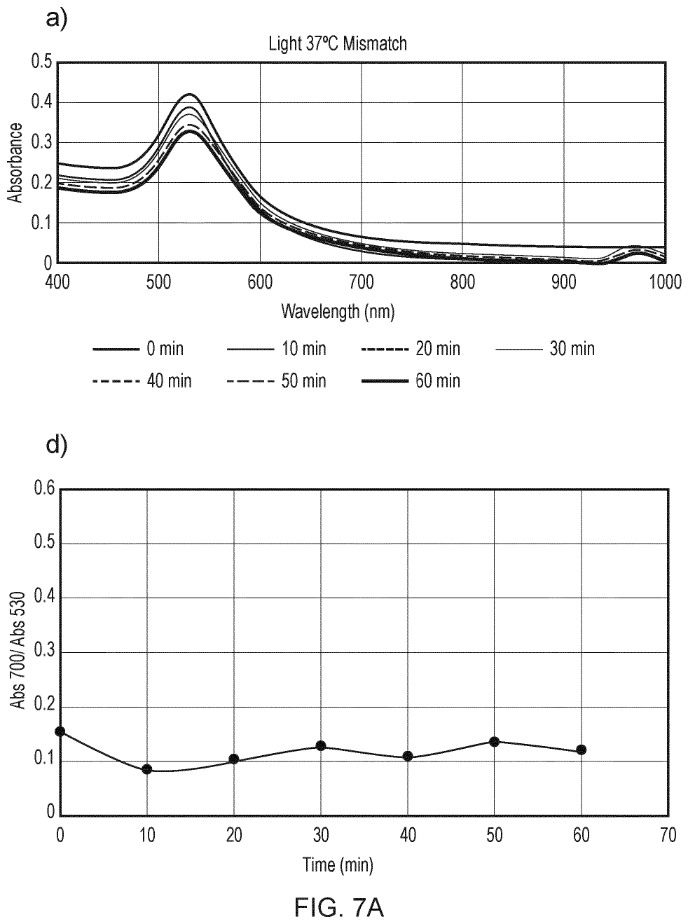

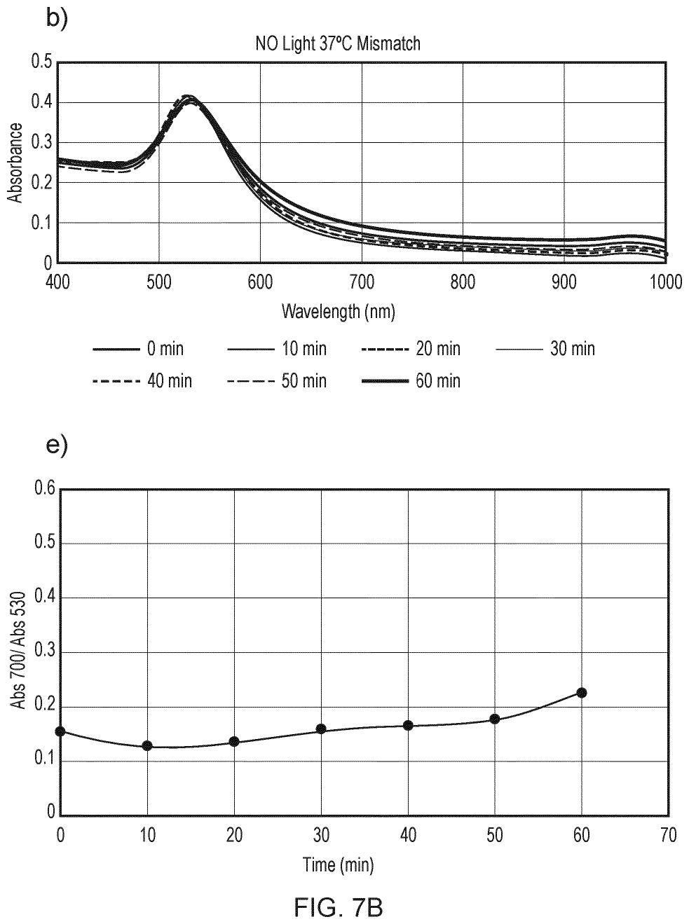

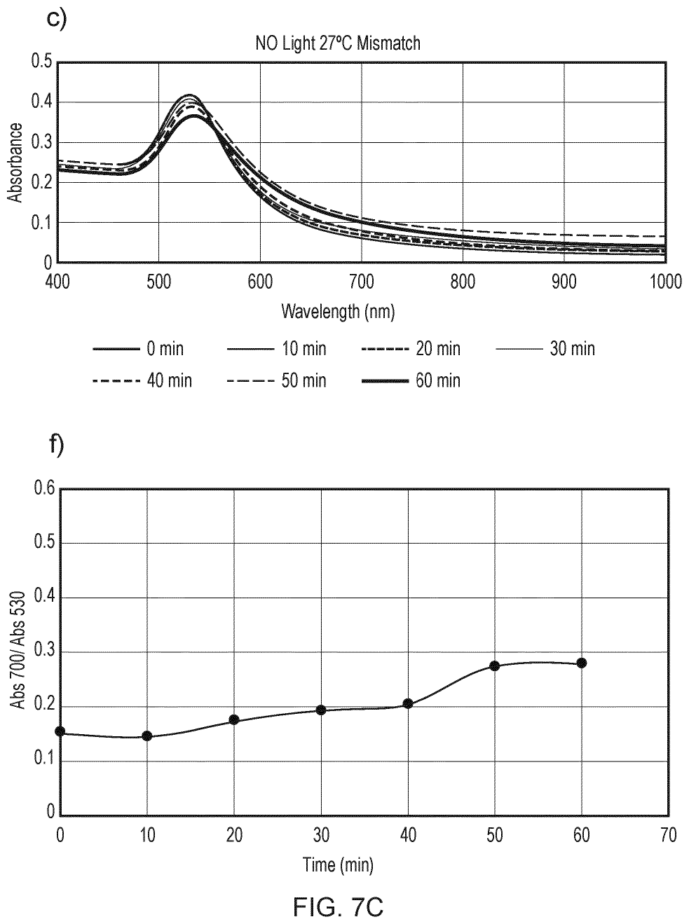

Three different experiments were carried out to see how stable formed agglomerates were with temperature. The results of FIG. 7A-7C show that the formed agglomerates were stable either at room temperature or higher temperature (37° C.) for at least 1 hour in the presence or absence of laser irradiation (600 nm).

### Example 8—Adaptability of Detection System to Multiple Analytes

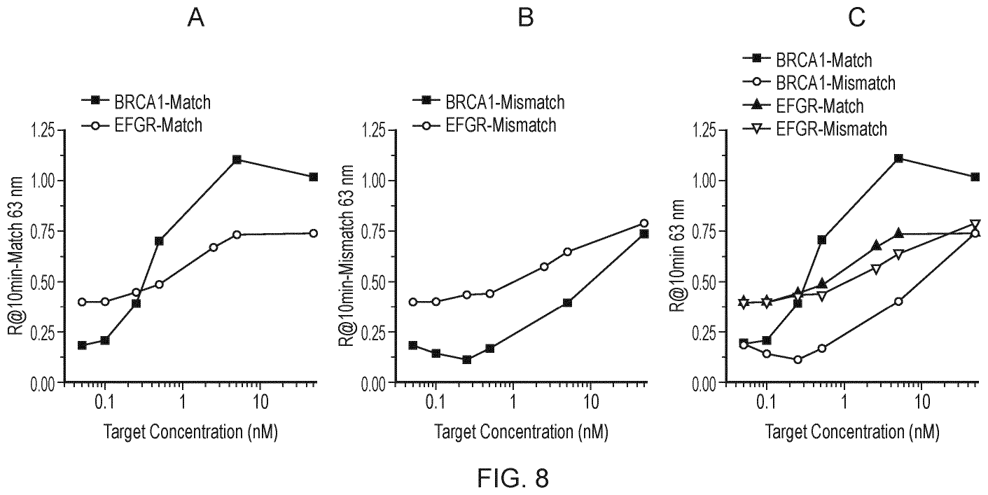

The detection system was tested on a number of different analytes that contained single nucleotide mismatches including naturally occurring mutations in the EGFR and BRCA1 genes. FIG. 8 demonstrates that, although there are clear differences between the kinetics of match and mismatch binding in the two different targets, both genes are able to be distinguished on the basis of the detection technology.

### Example 9—RNA Analyte Detection

Using a combination of 23 nt and 70 nt RNA analytes, the ability of the nanoparticle-probes was tested using 2″F-modified nucleic acid sequences.

In the first experiments 23 nt RNA analyte and 50 nm nanoparticles were used (see Table 1). Rapid differences in visual colour were observed in about 15 mins with a single base mismatch analyte (Data not shown).

TABLE 1
RNA analyte detection with 23 nt RNA analyte and 50 nm NPs

| | | | Hybrid- | | |
|---|---|---|---|---|---|
| WT | MUT F | PBS | isation | Match | Mismatch |
| (0.3486 mM) | (0.327 mM) | (10 mM) | Buffer | (1 μM) | (1 μM) |
| 72 μL | 76 μL | 327 μL | 25 μL | 2.5 μL | — |
| 72 μL | 76 μL | 327 μL | 25 μL | — | 2.5 μL |

The same experiments were repeated but using smaller nanoparticle size (25 nm) and 70 nt RNA analyte (see Table 2). There was a clear difference in optical colour in the presence of the analyte although this process was slower than in the first experiments (Data not shown).

TABLE 2
RNA analyte detection with 70 nt RNA analyte and 25 nm NPs

| | | | Hybrid- | | |
|---|---|---|---|---|---|
| WT | MUT F | PBS | isation | Match | Mismatch |
| (0.3486 mM) | (0.327 mM) | (10 mM) | Buffer | (1 μM) | (1 μM) |
| 72 μL | 76 μL | 302 μL | 50 μL | 2.5 μL | — |
| 72 μL | 76 μL | 302 μL | 50 μL | — | 2.5 μL |

### Example 10—Target Selection and Primer Design

Based on the experimental evidence provided herein, contiguous but non-overlapping primers targeting the E-protein gene were designed. It is expressly contemplated herein that other non-conserved sequences in other genes (e.g. the N-protein gene of SARS-CoV-2) could also be used for this purpose. The criteria for primer design disclosed herein can be applied when designing primers for use in the detection of other nucleic acids, for instance when designing primers for the detection of the nucleic acid of another pathogen.

The reasons for choosing the E-gene as the initial target were:
1. It is non-conserved between members of the coronavirus family i.e. it is specific and less likely to cross-react with closely related viral sequences.
2. It is located a 3″-end of the genome and therefore is likely to be highly abundant in its sgmRNA-form.
3. Although different technology, comparative qRT-PCR studies have found that higher sensitivity was obtained with E-gene detection than other genes (Corman et al).
4. The E gene is small (228 nt) making it practical to create a synthetic RNA genomic positive strand and complementary negative strand sgmRNA for optimisation of the technology without the need for using viral-derived or DNA analytes for experiments.

It is expressly contemplated herein that other sequences in SARS-CoV-2 may be targeted. For instance, reasons 1 and 2 above apply to the N-gene of SARS-CoV-2.

The initial primers for proof-of-principle experiments were chosen on the basis of being contiguous 20-mer sequences covering the E-gene sequence. It is desirable that the primers have the following properties:
1. Specificity for the SARS-CoV-2 virus, although certain cross-reactivity could be tolerated if unlikely to be a potential contaminant under the indications envisaged. For example, cross-reactivity with bat SARS or a plant-derived sequence could be tolerated.
2. No significant complementarity with other probe sequences in the test which could generate false-positives.
3. Melting temperatures (TM) ideally between 55-68° C. although other temperatures could be used.

Table 3 characterises the target sequences and capture probes designed by the present inventors. Two SARS-CoV-2 target sequences were selected: a 235 nt viral sequence and a 108 nt subgenomic E gene sequence. Three groups of capture probes were designed. E1-E5 are complementary to the one terminus of the 235 nt viral sequence. E12-E16 are complementary to the other terminus of the 235 nt viral sequence. E7-E11 and E17 are complementary to the 108 nt subgenomic E gene sequence. Table 4 provides the oligonucleotide sequences of these capture probes.

All primers have i) 5′-thiol modification for attachment to NPs and C6 spacer and ii) 10(t) 5′ tail upstream of complementary 20-mer sequence.

At least some of the primers E #1-6 and E #12-17 are contiguous and reverse complementary to the positive-strand RNA sequence of the E-gene of the SARS-CoV-2 sequence (ref seq NC 045512.2).

Primers E #R7-R11 and E17 target the negative strand sgmRNA of the E gene.

TABLE 3
Oligonucleotide target sequences and capture probes

| | | | | |
|---|---|---|---|---|
| Name | Oligonucleotide Sequence (5′->3′) | SEQ ID | Length | Tm |

Target Sequences

| | | | | |
|---|---|---|---|---|
| 235 | gggauguacucauucguuucggaagagacagguacguuaauaguu | 30 | 235 | |
| nt | aauagcguacuucuuuuucuugcuuucgugguauucuugcuaguu | | | |
| | acacuagccauccuuacugcgcuucgauugugugcguacugcugc | | | |
| | aauauuguuaacgugagucuuguaaaaccuucuuuuuacguuuac | | | |
| | ucucguguuaaaaaucugaauucuucuagaguuccugaucuucug | | | |
| | gucuaaguuu | | | |
| 108 | uuagaccagaagaucaggaacucuagaagaauucagauuuuuaac | 31 | 108 | 73.5° C. |
| nt | acgagaguaaacguaaaaagaagguuuuacaagacucacguuaac | | | |
| | aauauugcagcaguacgc | | | |

Capture Probes for 235 nt Sequence

| | | | | |
|---|---|---|---|---|
| E1 | SH-C6-tttttttttt-aca-caa-tcg-aag-cgc-agt-a | 32 | 29 | 61° C. |
| E2 | SH-C6-tttttttttt-agg-atg-gct-agt-gta-act-ag | 33 | 30 | 55° C. |
| E3 | SH-C6-tttttttttt-caa-gaa-tac-cac-gaa-agc-a | 34 | 29 | 60° C. |
| E4 | SH-C6-tttttttttt-aga-aaa-aga-agt-acg-cta-tt | 35 | 30 | 55° C. |
| E5 | SH-C6-tttttttttt-aac-tat-taa-cgt-acc-tgt-ctc- | 36 | 32 | 55° C. |
| | t | | | |
| E12 | SH-C6-tttttttttt-tta-gac-cag-aag-atc-agg-aa | 37 | 30 | 59° C. |
| E13 | SH-C6-tttttttttt-ctc-tag-aag-aat-tca-gat-tt | 38 | 30 | 54° C. |
| E14 | SH-C6-tttttttttt-tta-aca-cga-gag-taa-acg-ta | 39 | 30 | 56° C. |
| E15 | SH-C6-tttttttttt-aaa-aga-agg-ttt-tac-aag-ac | 40 | 30 | 55° C. |
| E16 | SH-C6-tttttttttt-tca-cgt-taa-caa-tat-tgc-ag | 41 | 30 | 58° C. |

Capture Probes for 108 nt Sequence

| | | | | |
|---|---|---|---|---|
| E7 | SH-C6-tttttttttt-gcg-tac-tgc-tgc-aat-att-gt | 42 | 30 | 62° C. |
| E8 | SH-C6-tttttttttt-taa-cgt-gag-tct-tgt-aaa-a | 43 | 29 | 55° C. |
| E9 | SH-C6-tttttttttt-ttc-ctt-ctt-ttt-acg-ttt-act-c | 44 | 32 | 58° C. |
| E10 | SH-C6-tttttttttt-ttt-cgt-gtt-aaa-aat-ctg-aat- | 45 | 32 | 59° C. |
| | t | | | |
| E11 | SH-C6-tttttttttt-ctt-cta-gag-ttc-ctg-atc-ttc- | 46 | 33 | 60° C. |
| | tg | | | |
| E17 | SH-C6-tttttttttt-gct-tcg-att-gtg-tgc-gta- | 47 | | |
| | ctg | | | |

Other

| | | |
|---|---|---|
| N2 | SH-C6-tttttttttt-ag tat tat tgg gta aac ctt | 48 |
| N3 | SH-C6-tttttttttt-tt gcc atg ttg agt gag agc | 49 |
| N4 | SH-C6-tttttttttt-ct tgt cct cga ggg aat tta | 50 |
| N6 | SH-C6-tttttttttt-gt agc caa ttt ggt cat ctg | 51 |
| N7 | SH-C6-tttttttttt-gg act gag atc ttt cat ttt | 52 |
| N8 | SH-C6-tttttttttt-ag ttc cta ggt agt aga aat | 53 |
| N9 | SH-C6-tttttttttt-ta ggg aag tcc agc ttc tgg | 54 |
| N17 | SH-C6-tttttttttt-ag ctg gtt caa tct gtc aag | 55 |
| N19 | SH-C6-tttttttttt-ag ttt ggc ctt gtt gtt gtt | 56 |
| N20 | SH-C6-tttttttttt-gc agc aga ttt ctt agt gac | 57 |
| LS- | SH-C6-tttttttttt-gg ttt gtt acc tgg gaa ggt | 58 |
| 20#1 | | |
| LS- | SH-C6-tttttttttt-ac aag aga tcg aaa gtt ggt t | 59 |
| 20#2 | | |
| LS- | SH-C6-tttttttttt-gt tcg ttt aga gaa cag atc t | 60 |
| 20#3 | | |

TABLE 4
Oligonucleotide sequences
of the capture probes

| | SEQ | |
|---|---|---|
| Primer | ID | |
| reference | NO: | Primer sequence |
| E1 | 1 | aca-caa-tcg-aag-cgc-agt-a |
| E2 | 2 | agg-atg-gct-agt-gta-act-ag |
| E3 | 3 | caa-gaa-tac-cac-gaa-agc-a |
| E4 | 4 | aga-aaa-aga-agt-acg-cta-tt |
| E5 | 5 | aac-tat-taa-cgt-acc-tgt-ctc-t |
| E12 | 6 | tta-gac-cag-aag-atc-agg-aa |
| E13 | 7 | ctc-tag-aag-aat-tca-gat-tt |
| E14 | 8 | tta-aca-cga-gag-taa-acg-ta |
| E15 | 9 | aaa-aga-agg-ttt-tac-aag-ac |
| E16 | 10 | tca-cgt-taa-caa-tat-tgc-ag |
| E7 | 11 | gcg-tac-tgc-tgc-aat-att-gt |
| E8 | 12 | taa-cgt-gag-tct-tgt-aaa-a |
| E9 | 13 | ttc-ctt-ctt-ttt-acg-ttt-act-c |
| E10 | 14 | ttt-cgt-gtt-aaa-aat-ctg-aat-t |
| E11 | 15 | ctt-cta-gag-ttc-ctg-atc-ttc-tg |
| E17 | 16 | gct-tcg-att-gtg-tgc-gta-ctg |
| N2 | 17 | ag tat tat tgg gta aac ctt |
| N3 | 18 | tt gcc atg ttg agt gag agc |
| N4 | 19 | ct tgt cct cga ggg aat tta |
| N6 | 20 | gt agc caa ttt ggt cat ctg |
| N7 | 21 | gg act gag atc ttt cat ttt |
| N8 | 22 | ag ttc cta ggt agt aga aat |
| N9 | 23 | ta ggg aag tcc agc ttc tgg |
| N17 | 24 | ag ctg gtt caa tct gtc aag |
| N19 | 25 | ag ttt ggc ctt gtt gtt gtt |
| N20 | 26 | gc agc aga ttt ctt agt gac |
| LS-20#1 | 27 | gg ttt gtt acc tgg gaa ggt |
| LS-20#2 | 28 | ac aag aga tcg aaa gtt ggt t |
| LS-20#3 | 29 | gt tcg ttt aga gaa cag atc t |

LS = Leader sequence

### Example 11—Analysis of Secondary Structure of Capture Probes

The secondary structures for each capture probe described in Table 3 were simulated and the free energies of those secondary structures 3 were calculated. It was observed that almost all capture probes form stable secondary structures at 25° C.

E4 did not form a stable secondary structure at 25° C. Surface functionalisation of nanoparticles with capture probe E4 through a “salt aging” method led to limited colloidal stability of these nanoparticles. Without wishing to be bound by theory, the present inventors theorised that the secondary structure of E4 impedes its efficient adsorption on the nanoparticles surface. The present inventors therefore theorise that the sequence of the capture probe, specifically whether stable secondary structures can be formed, is important for the stability of the nanoparticle-probe. Re-dispersion of these nanoparticles in SDS 0.01% allowed for a full recovery of the nanoparticles' stability.

### Example 12—Analysis of Secondary Structure of Target Sequences

The secondary structure of the viral 235 nt and subgenomic 108 nt target sequences were simulated. It was observed that each target sequence can form structures of relatively low energies at 25° C. 1M NaCl. The viral 235 nt secondary structure had a free energy of −77.17 kcal/mol, whereas the subgenomic 108 nt secondary structure had a free energy of −25.78 kcal/mol.

### Example 13—Simulation of Secondary Structures of Hybrids Comprising 235 nt and 108 nt and Capture Probes

In order to obtain more detailed information on the binding between capture and target sequences, the most probable secondary structures at 25° C. were simulated. The secondary structures of a hybrid of 235 nt viral sequence and E1, E2, E3, E4 or E5 capture probe were simulated. The energies for all hybrids were comparable, varying from −105.59 to −100.41 kcal/mol.

Similar secondary structures were observed for the hybrid of the 235 nt viral sequence and E12, E13, E14, E15 or E16 capture probe were observed. The free energy varied slightly more, from −110.46 to −95.28 kcal/mol. E12-E16 bind to the opposite terminus of the 235 nt viral sequence as compared with E1-E5.

It was also observed that the 108 nt subgenomic sequence forms stable hybrids with the capture probe E7, E8, E9, E10 and E11. The free energy varied from −67.12 to −46.78 kcal/mol.

### Example 14—Detection of 108 nt Subgenomic Sequence

To evaluate the detection of the 108 nt subgenomic sequence, the assay composition was tested using 1, 2, 3, 4 or 5 NP@DNA batches, wherein each batch comprises a different NP@DNA population. The total number of particles in each assay was kept constant (expressed as molar concentration of Au⁰-150 μM). For each assay composition, the following concentrations of target sequence were used: 10000 pM, 1000 pM, 100 pM, 10 pM, 1 pM, 0.1 pM. The mixtures were incubated for 48 hours.

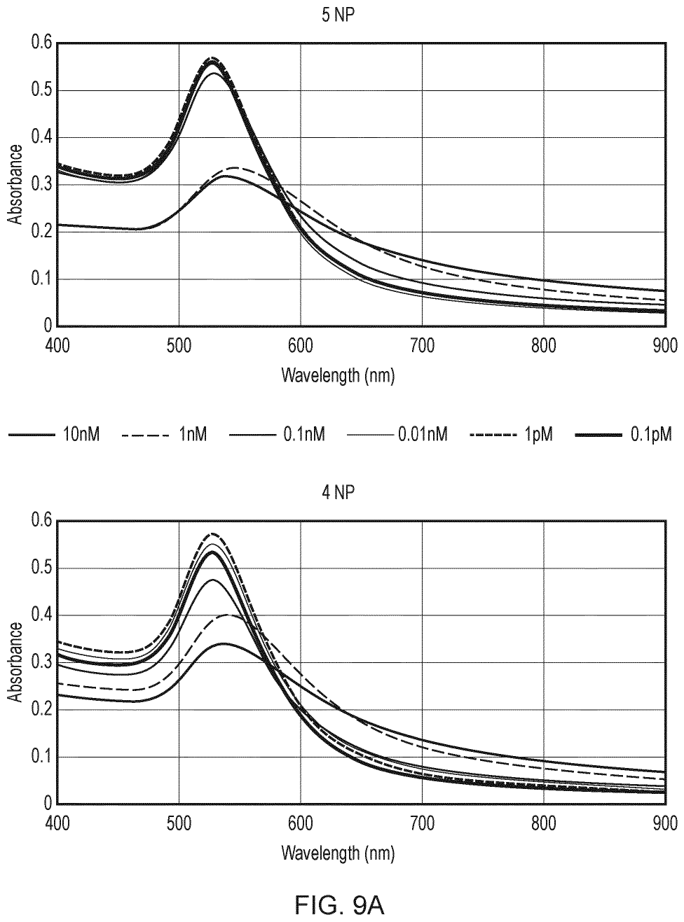

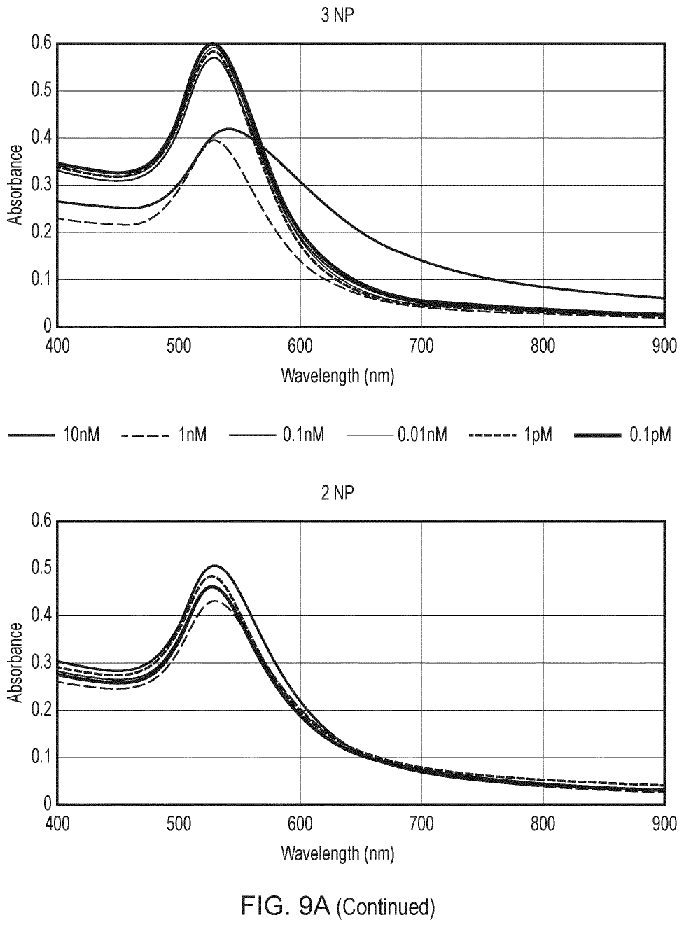

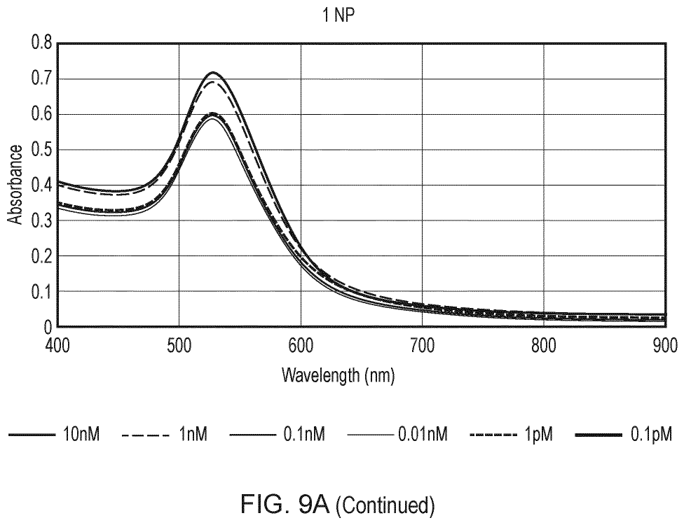

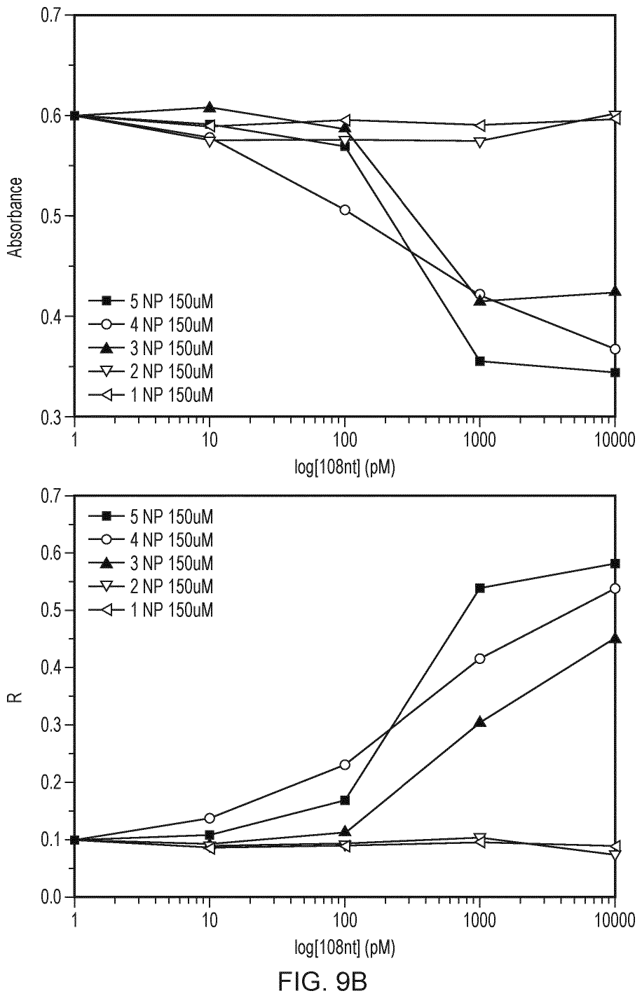

FIG. 9A shows UV-Vis-NIR spectra of the solutions at different concentrations of target for the assays of 5, 4, 3, 2 and 1 NPs. It was observed that the spectra remained unchanged over the entire range of target concentration for batches containing 1 and 2 NPs. Oppositely, for the assay containing 3, 4 and 5 batches, an abrupt change in UV-Vis-NIR spectra was observed. This was especially visible for samples containing 4 and 5 batches.

A more detailed analysis of the spectral data showed that the mixtures containing 1 and 2 batches remained stable over the entire range of target concentration and only mixtures containing 3, 4, and 5 batches underwent aggregation above 100 pM (FIG. 9B). The spectral data were also confirmed by naked-eye inspection, which showed that the lower the target concentration the lower is the colour intensity of the samples. The colour change was not visible for 1 and 2 batches (data not shown). The colour change was visible for 3, 4, and 5 batches with the effect being more pronounced for 4 and 5 batches than for 3 batches.

The overall concentration of nanoparticles in this experiment was kept fixed at 150 μM. Accordingly, the concentration of each population can be calculated based on the number of batches used. For instance, for 2 batches, the concentration of each population was 75 μM. For 3 batches, the concentration of each population was 50 μM.

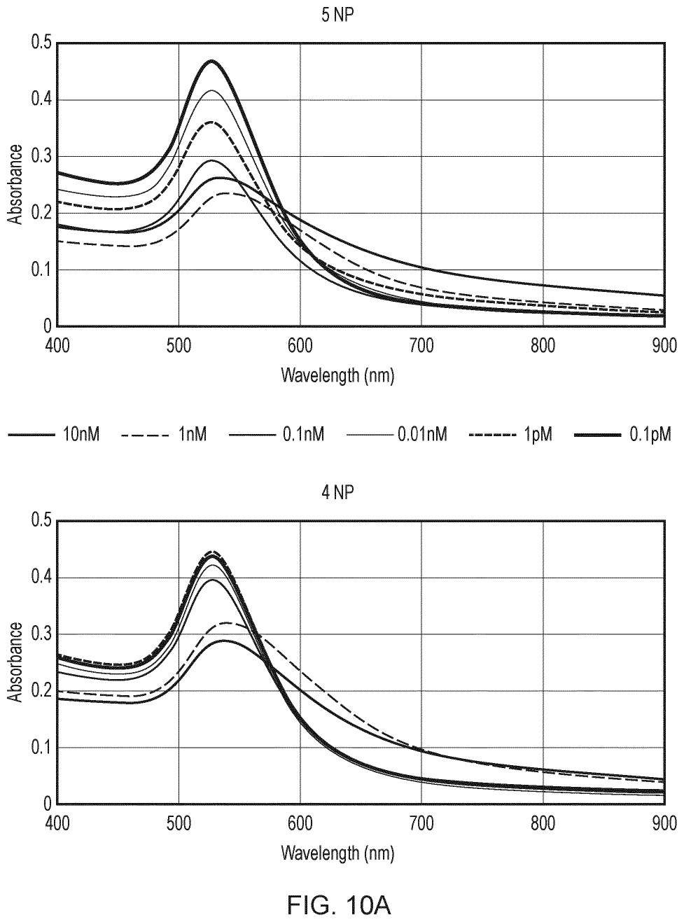

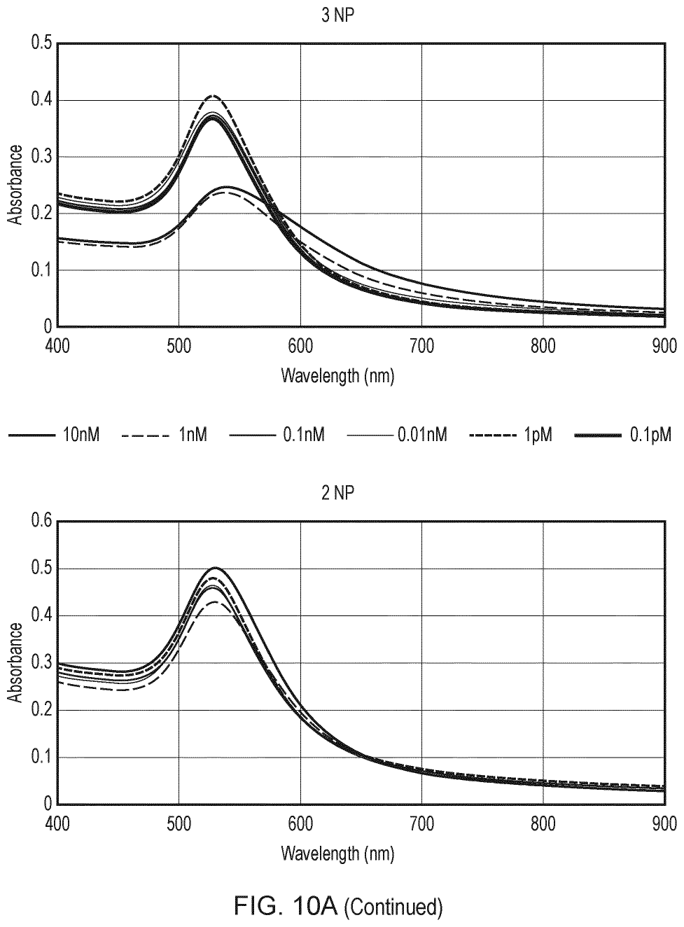

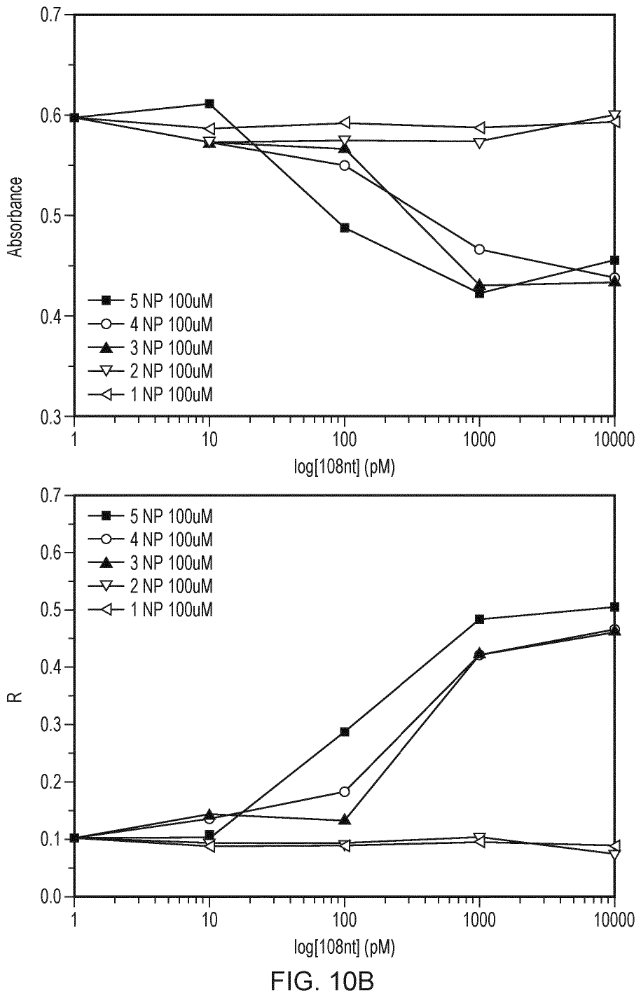

In order to check whether the assay performance could be improved, the experiments of Example 14 were repeated by changing one parameter only. The total concentration of the nanoparticles was set to 100 μM (FIGS. 10A-B). As in the previous experiments, the plasmon band of samples containing 1 or 2 batches remained invariant to the change of analyte concentration (FIG. 10A). But, for 3, 4 and 5 batches the optical properties changed with increasing analyte concentration.

These data also indicate that by decreasing the total concentration of nanoparticles (or molar ratio of nanoparticles to target) it is possible to improve the limit of detection down to 100 μM for a 5-batch system (FIG. 10B). Therefore by varying the concentration of probes to analyte the sensitivity of the system can be increased.

### Example 15—Detection of 108 nt Subgenomic Sequence in Assays Using Different Total Numbers of Particles

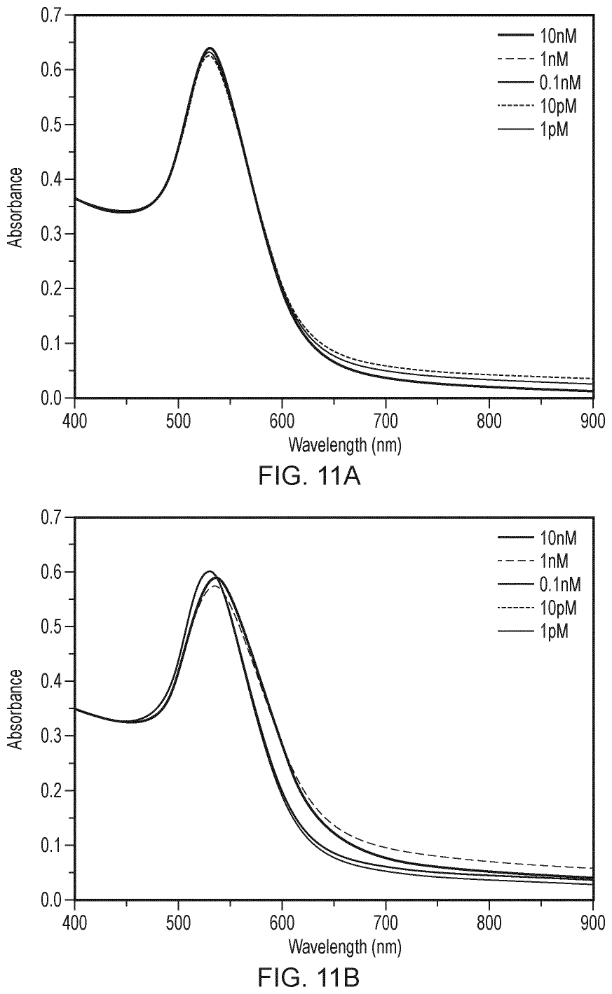

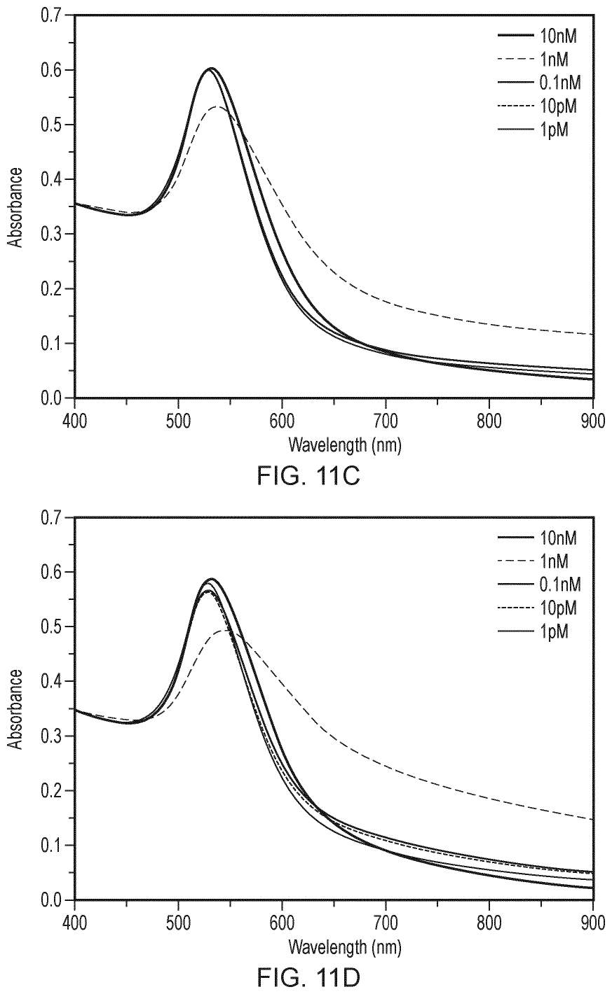

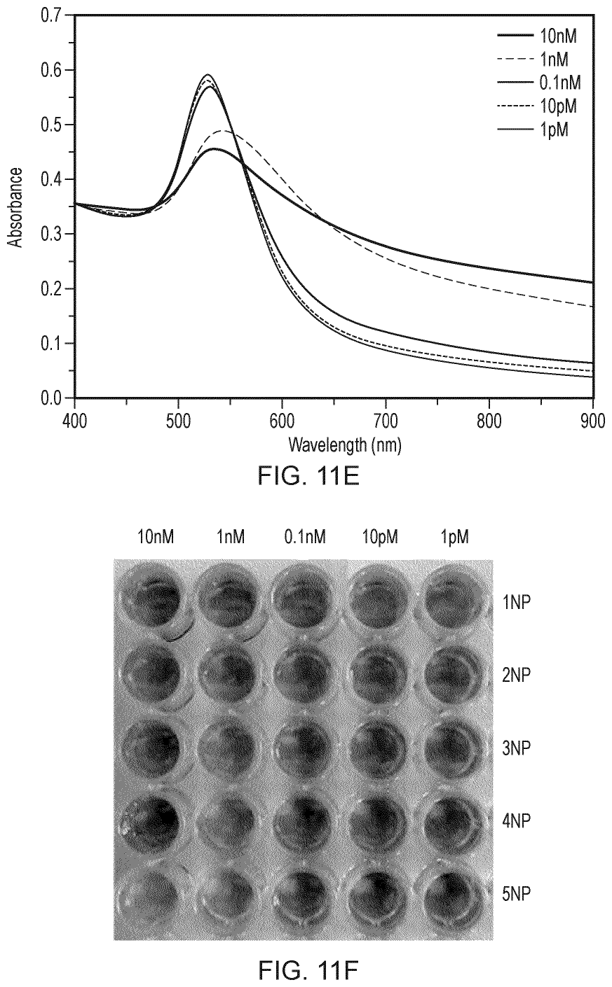

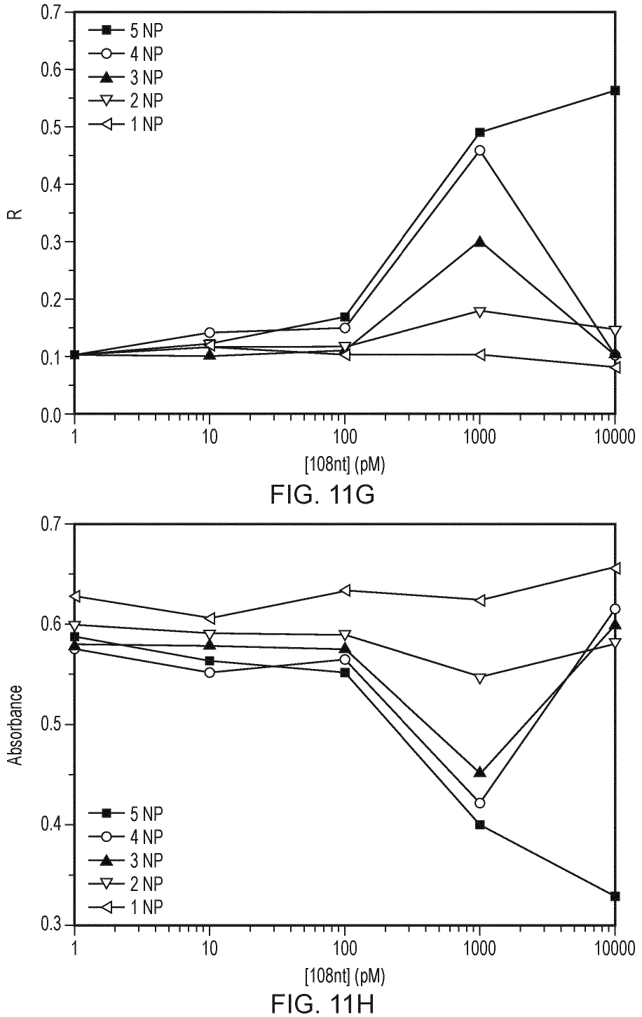

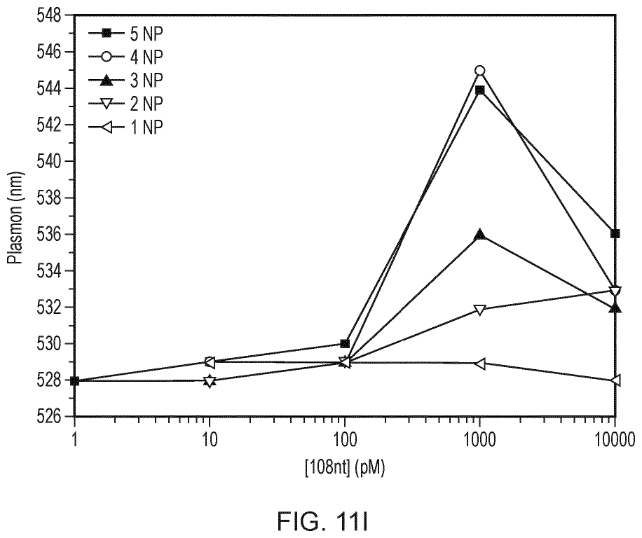

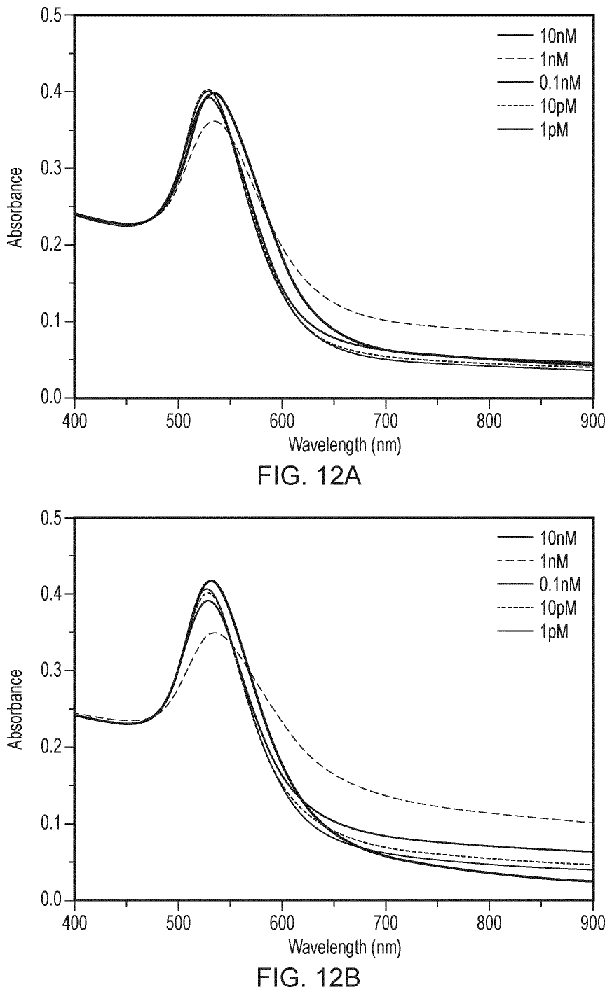

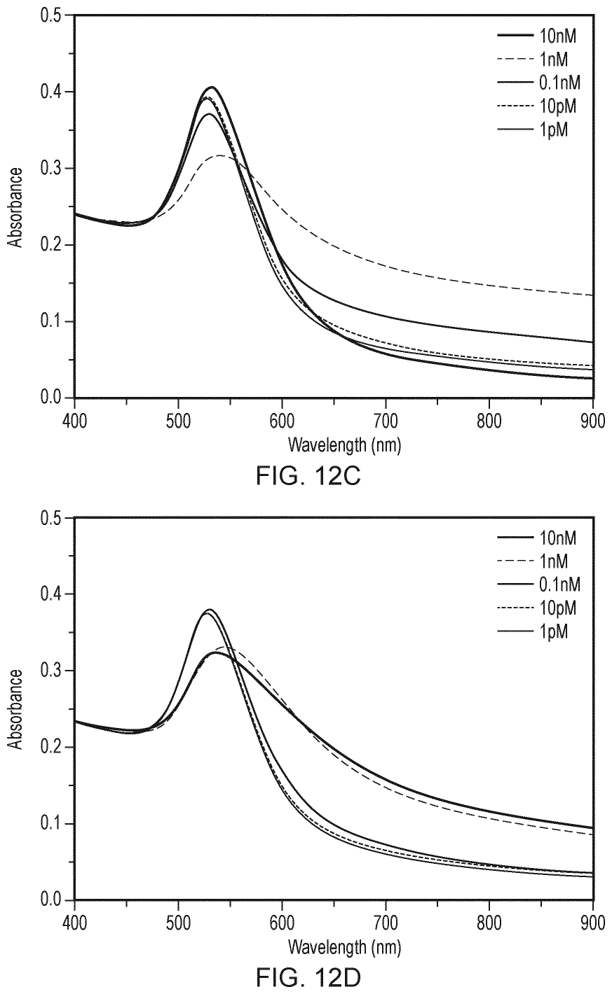

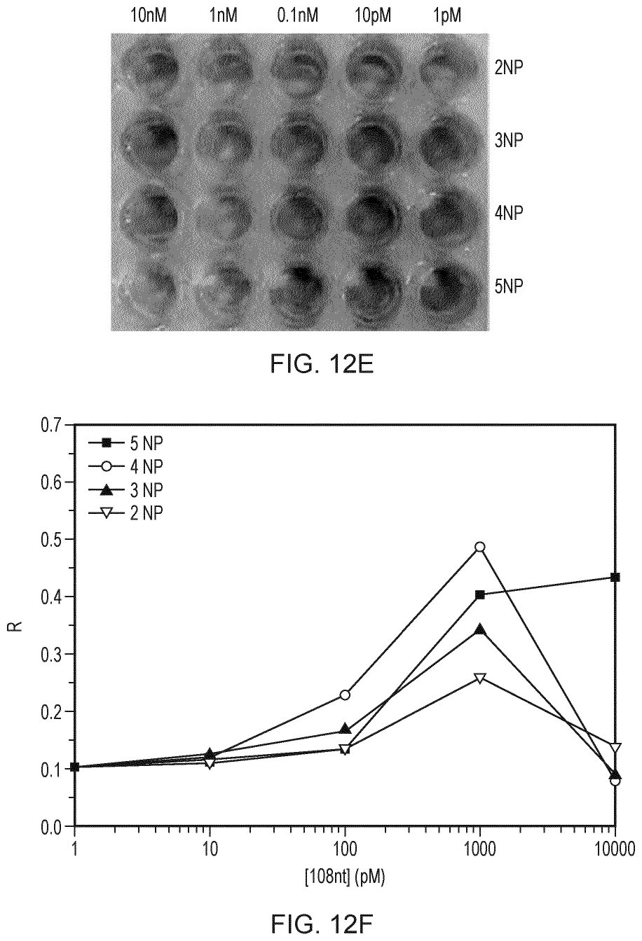

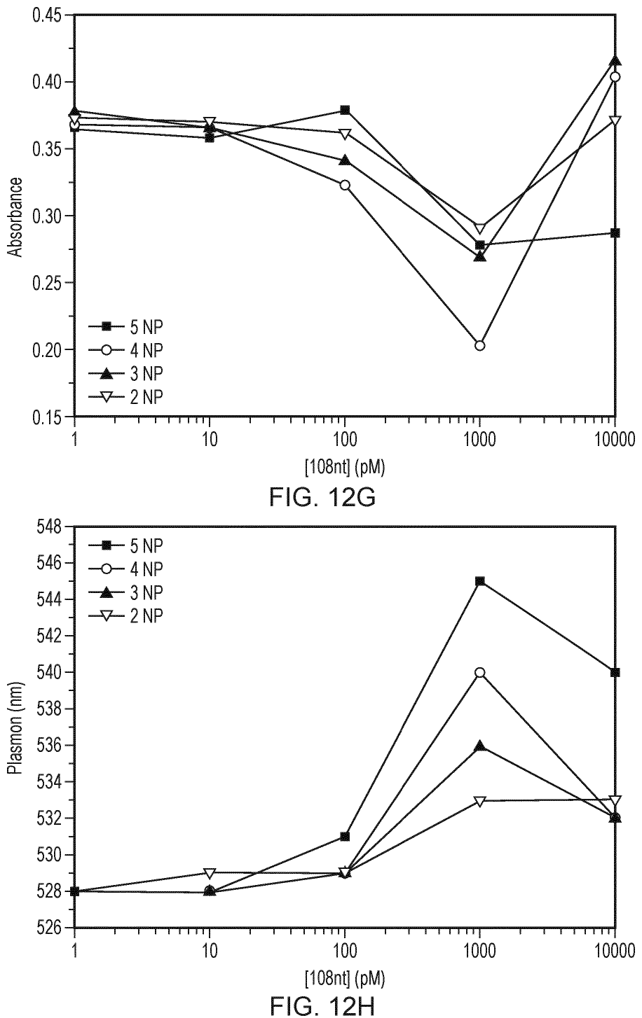

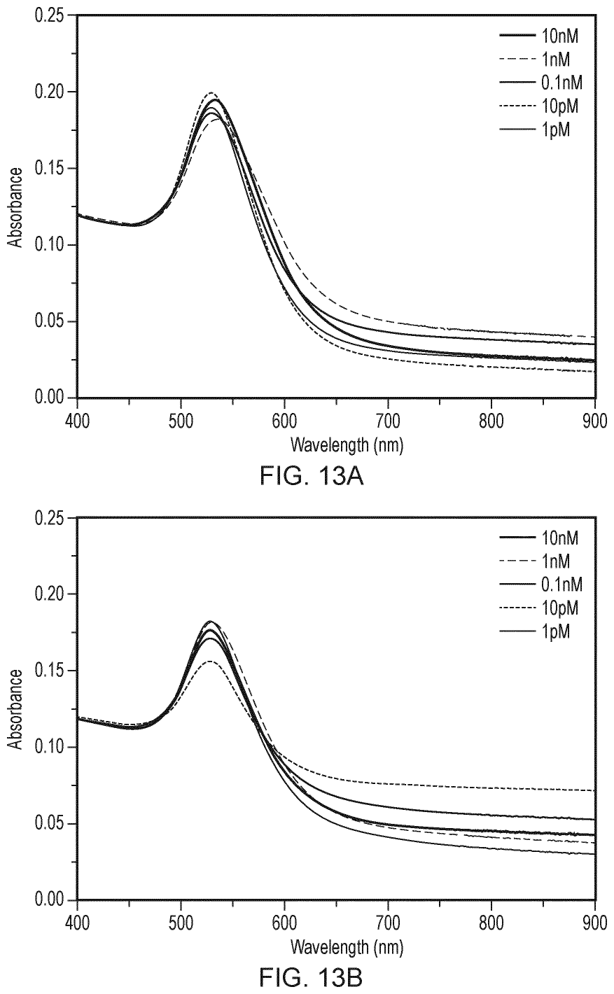

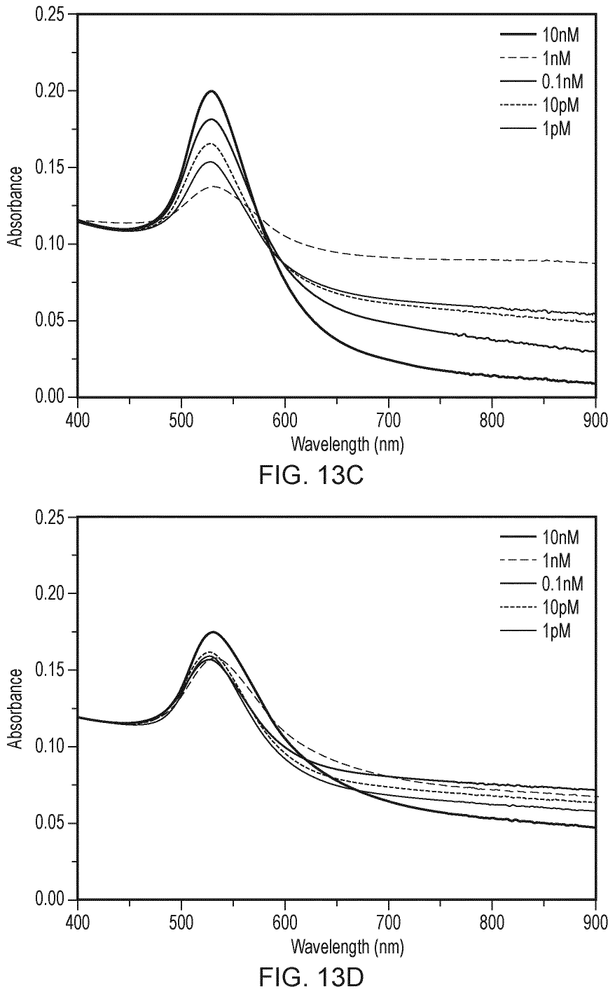

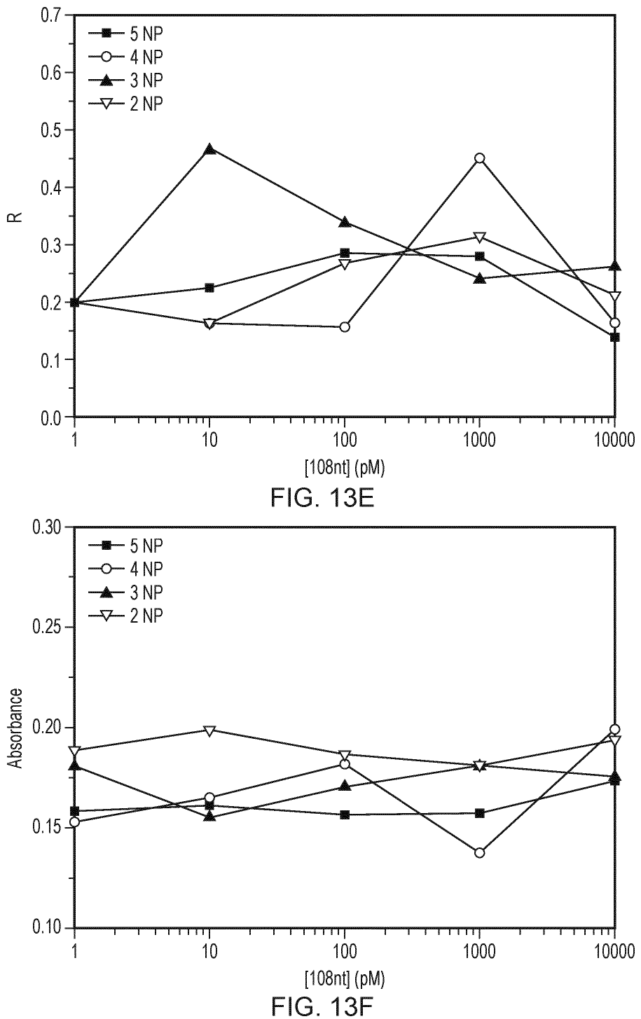

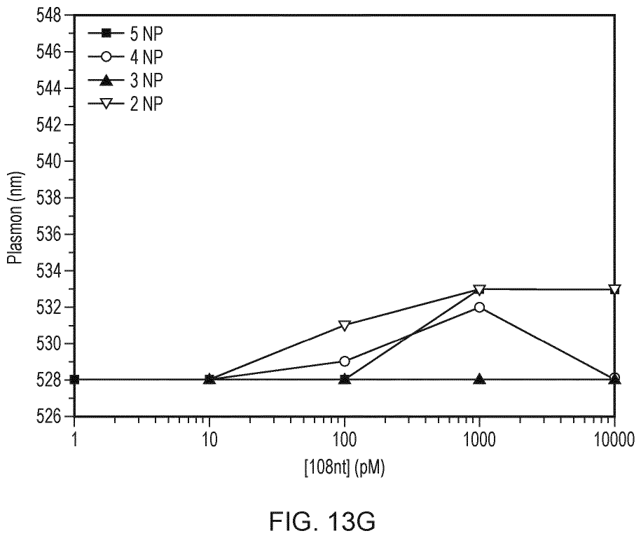

To evaluate the detection of the 108 nt subgenomic sequence, the assay composition was tested using 1, 2, 3, 4 or 5 NP@DNA batches, wherein each batch comprises a different NP@DNA population. The total number of particles in each assay (expressed as molar concentration of)Au⁰ was kept fixed to 150 μM (FIG. 11), 100 μM (FIG. 12), or 50 μM (FIG. 13). For each assay composition, the following concentrations of target sequence were used: 10000 pM, 1000 pM, 100 pM, 10 pM, 1 pM. The incubation time of 48 h was kept constant in each sample.

FIGS. 11A-E show UV-vis-NIR spectra of the solutions at different concentration of target for the assays of 1, 2, 3, 4 and 5 batches, respectively. The present inventors observed that for batches containing 1 and 2 types of nanoparticles (FIGS. 11A and 11B) the spectra remained unchanged over the entire range of target concentration. Oppositely, for the assay containing 3, 4 and 5 batches (FIGS. 11C, 11D and 11E), an abrupt change in UV-vis-NIR spectra was observed. This was especially visible for samples containing 4 and 5 batches.

The spectral data were also confirmed by naked-eye inspection (FIG. 11F). A more detailed analysis of the spectral data showed that the mixtures containing 1 and 2 batches remained stable over entire range of target concentration and only the mixtures containing 3, 4, and 5 batches experienced aggregation at 1 nM of target concentration. FIGS. 11G, 11H and 11I represent the change of aggregation degree, absorbance, and plasmon band shift, respectively versus target concentration for different numbers of nanoparticles batches. For the batch of 5 NPs (150 μM) the target concentration of 10 and 1 nM was detected. For batches of 3 and 4 NPs the target concentration of 1 nM was detected. At higher target concentrations (10 nM), the NPs remained stable.

The overall concentration of nanoparticles in this experiment was kept fixed at 150 μM. Accordingly, the concentration of each population can be calculated based on the number of batches used. For instance, for 2 batches, the concentration of each population was 75 μM. For 3 batches, the concentration of each population was 50 μM.

To check whether the assay performance can be improved, the above-described experiments were repeated by changing one parameter only. The total concentration of the nanoparticles that was set to 100 μM (FIG. 12). The aggregation of nanoparticles is more pronounced at 1 nM of target for samples containing more than two batches. Again, at a higher amount of target (10 nM) the particles remain stable, leading to negative results due to a lack of aggregation. This is because of surface saturation of each nanoparticle by target molecules.

The above-described experiments were repeated by decreasing the total concentration of nanoparticles to 50 μM (FIG. 13). It was observed that this concentration of nanoparticles was sub-optimal, as no visible difference was observed for experiments using 2 or more batches of nanoparticles.

The inability to detect higher amount of target is related to the saturation of each nanoparticles by target molecules, inhibiting thus the aggregation. In general, proper functioning of a colloidal assay depends on molar ratio of target to nanoparticles. At low values of the ratio a small fraction of particles are aggregated while vast majority of nanoparticles remains dispersed, causing no change of the colour. At higher values of the ratio, target molecules saturate surface of nanoparticles, again, causing no aggregation. Table 4 provides the values of molar ratios of target to nanoparticles.

TABLE 5
Ratio of target molecules and number of gold nanoparticles
for different concentration of Au₀ and different
concentrations of target used in the assays

| [108 nt] (pM) | 150 μM Au⁰ | 100 μM Au⁰ | 50 μM Au⁰ |
|---|---|---|---|
| 10000 | 55.67 | 83.51 | 167 |
| 1000 | 5.57 | 8.35 | 16.7 |
| 100 | 0.56 | 0.84 | 1.67 |
| 10 | 0.06 | 0.08 | 0.17 |
| 1 | 0.006 | 0.008 | 0.017 |

### Example 16—Detection of 235 nt Viral Sequence Using Sonication to Fragment the Analyte

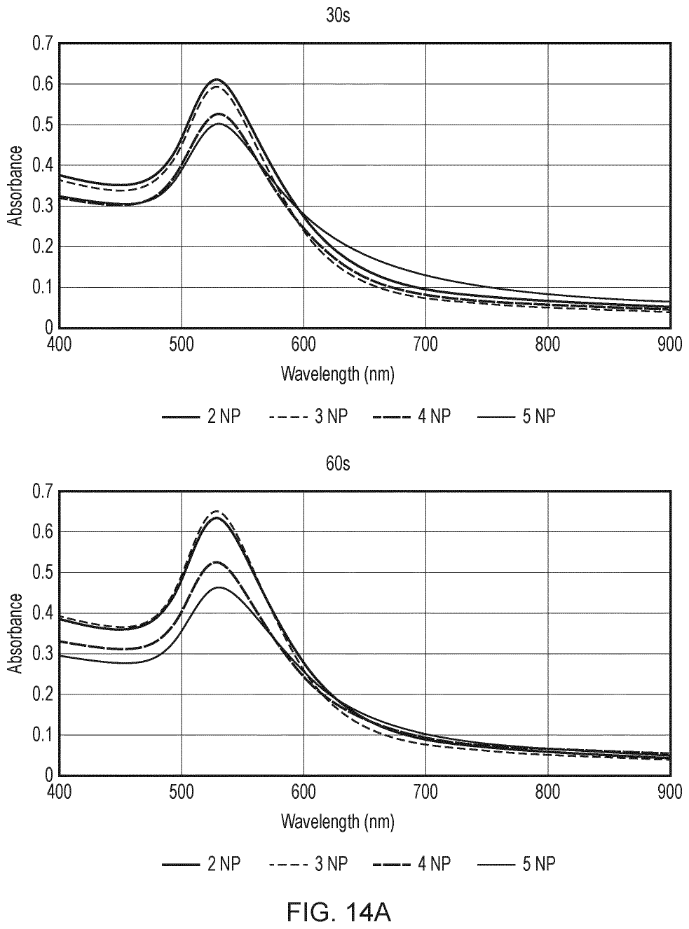

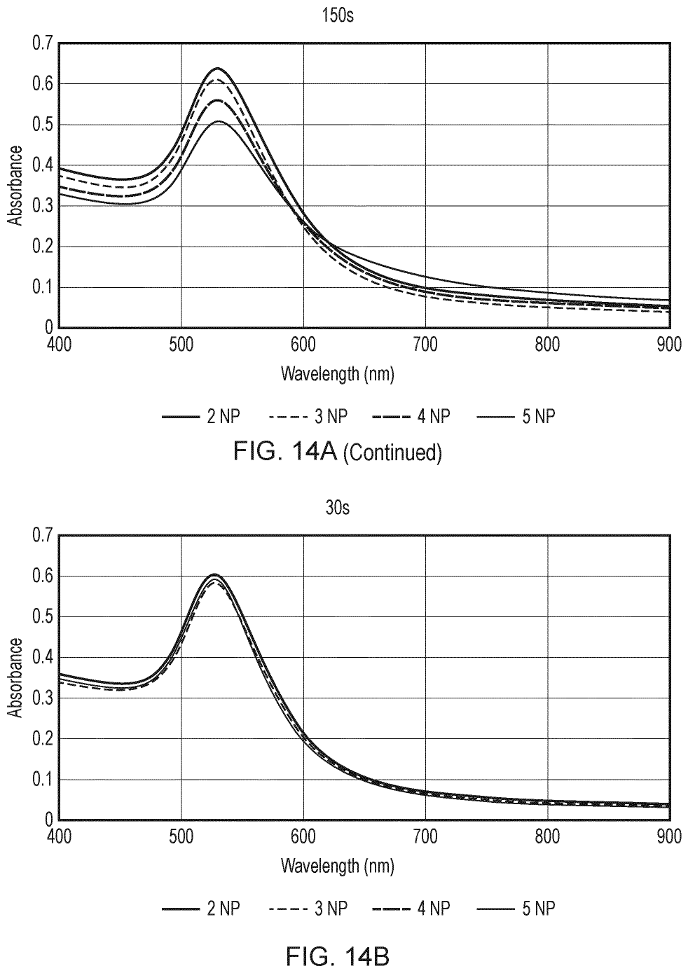

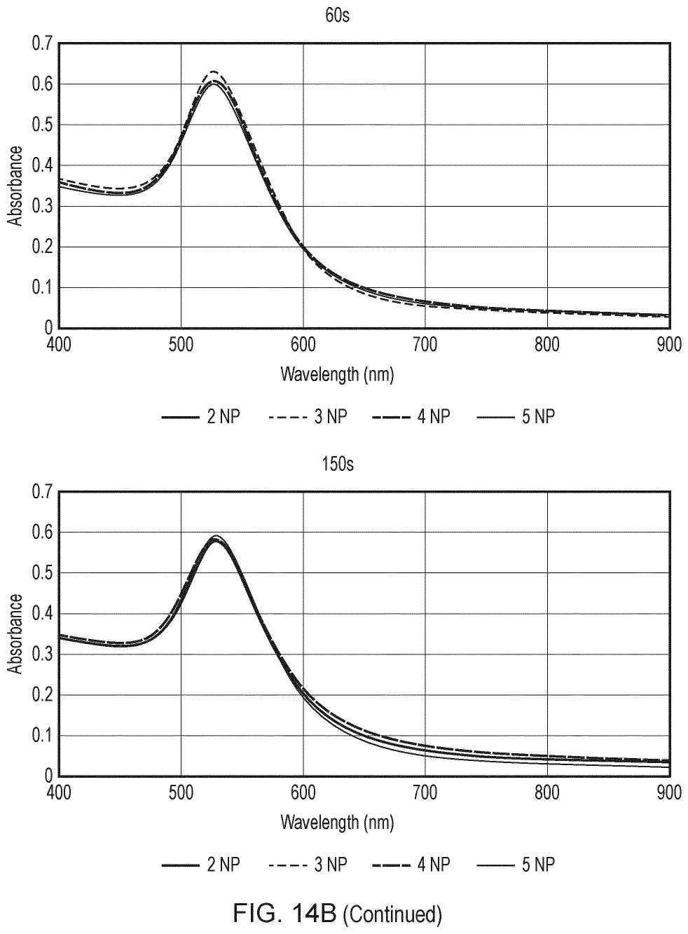

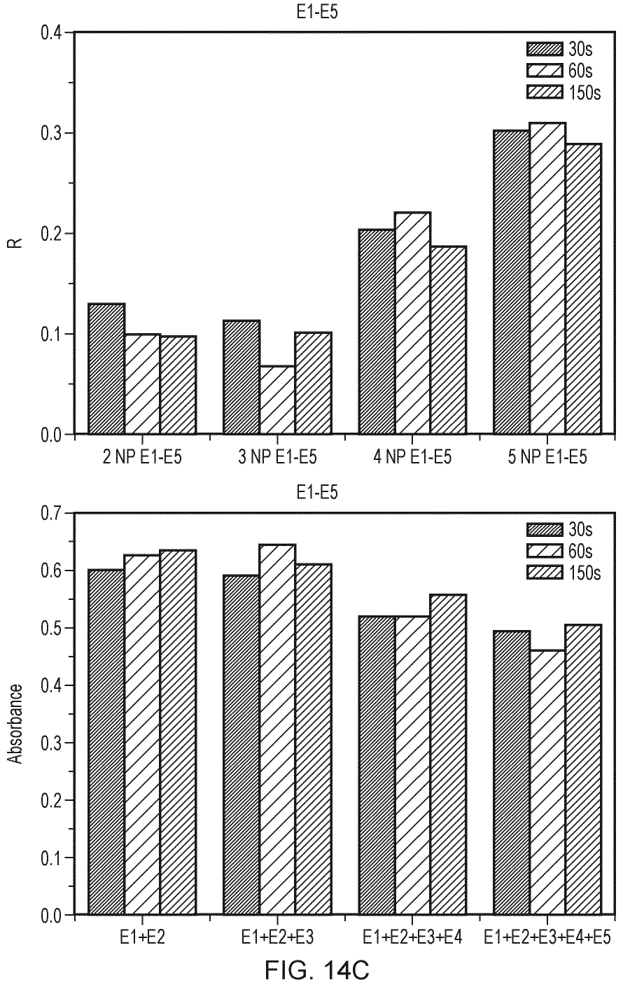

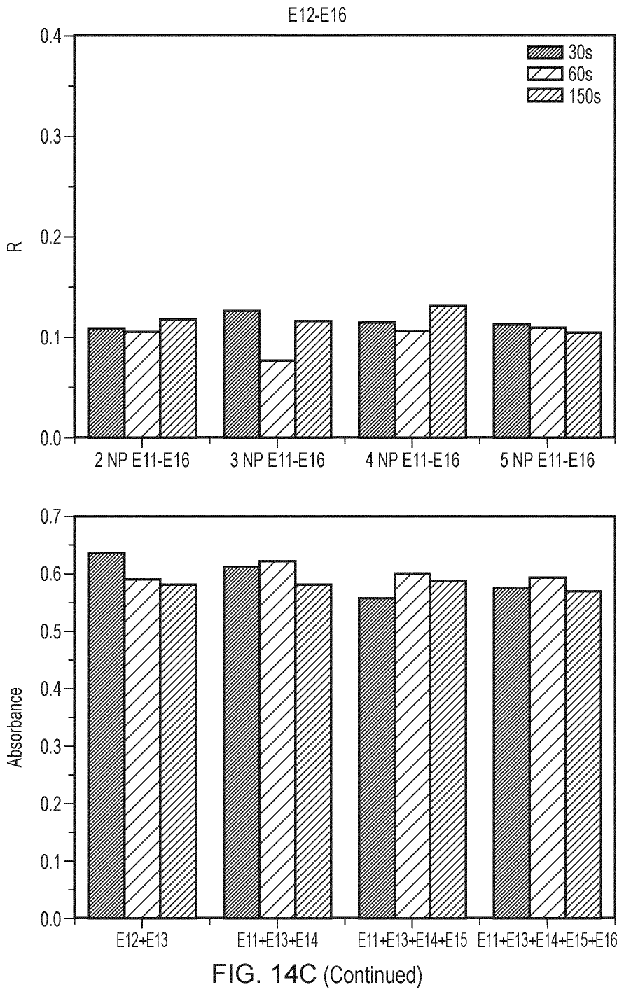

For the detection of 235 nt target sequence, two different sets of capture were employed: capture probes E1-E5 from the 5′ end to the middle of the target sequence and capture probes E12-E16 from the middle of the 3′ end of the target sequence. To induce fragmentation of the 235 nt sequence, the solution containing NPs (150 μM), target sequence 235 nt (1 nM) and NaCl 0.3 M was placed in sonic bath (1200 W) for different times: 30, 60, and 150 s. Subsequently the samples were incubated for 48 hours. Results are shown in FIGS. 14A and 14B for E1-E5 and E12-E16, respectively, and FIG. 14C.

These data suggest that systems containing 1 and 2 batches of gold nanoparticles remain invariant regardless of the sonication time. For systems containing 4 and 5 batches, the effect of sonication is more pronounced. Interestingly, the hybridization segment of capture probes with target sequence seems to be important for this type of detection strategy. While the sonication induces aggregation of NPs stabilized with capture probes E1-E5, it has no effect on stability when NPs stabilized with E12-E16 are used (FIG. 14C).

It is expressly contemplated herein that these experiments could be repeated for sonication times of 0, 60 and 300 sec. Without wishing to be bound by theory, the present inventors theorise that prolonged sonication will cause fragmentation of the target sequence, and thus no aggregation.

These experiments suggested that the length of the target analyte is critical for the plasmon resonance, as a colour change was demonstrated for the 108 nt subgenomic target analyte but not for the 235 nt viral target analyte. The present inventors used sonication to break up the 235 nt viral target analyte.

It is expressly contemplated herein that a fragmentation step (e.g. via sonication or other suitable process) may be necessary for long analytes, such as the genomic RNA of SARS-CoV-2 (>29 kb). It is expressly contemplated herein that the effectiveness of sonication may be sequence specific. For instance, the present inventors found that E1-5 were sensitive to sonication but E12-E16 were not. The effect of sonication was increased for batches of more than 4 or more than 5 NPs, but showed little difference when using 3 NPs.

### Example 17—Detection of 235 nt Sequence

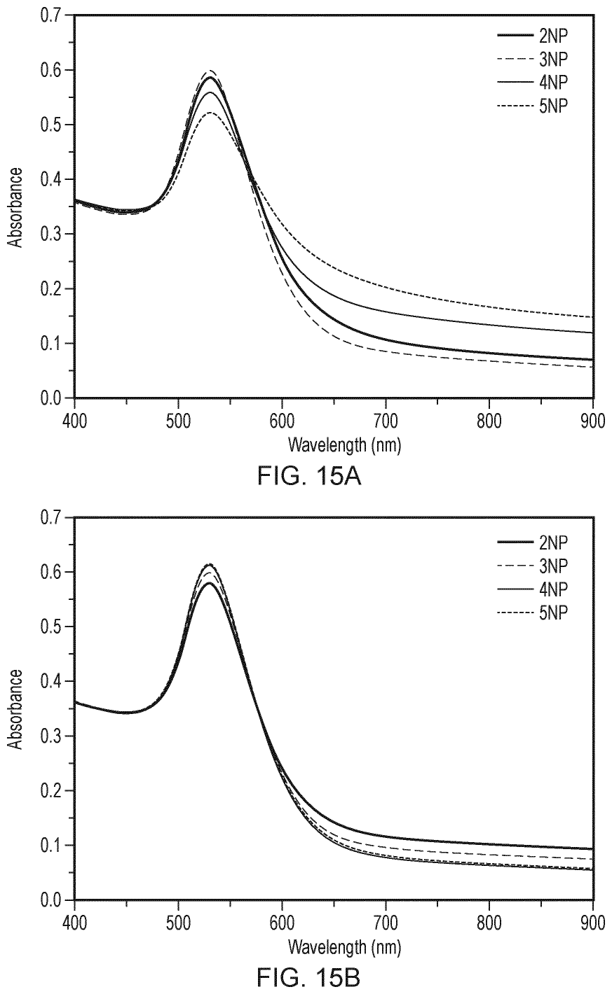

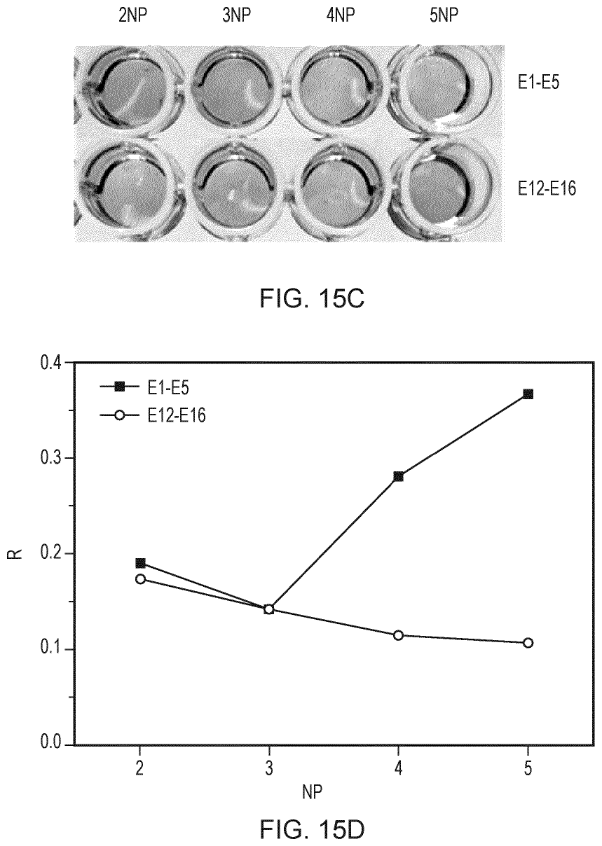

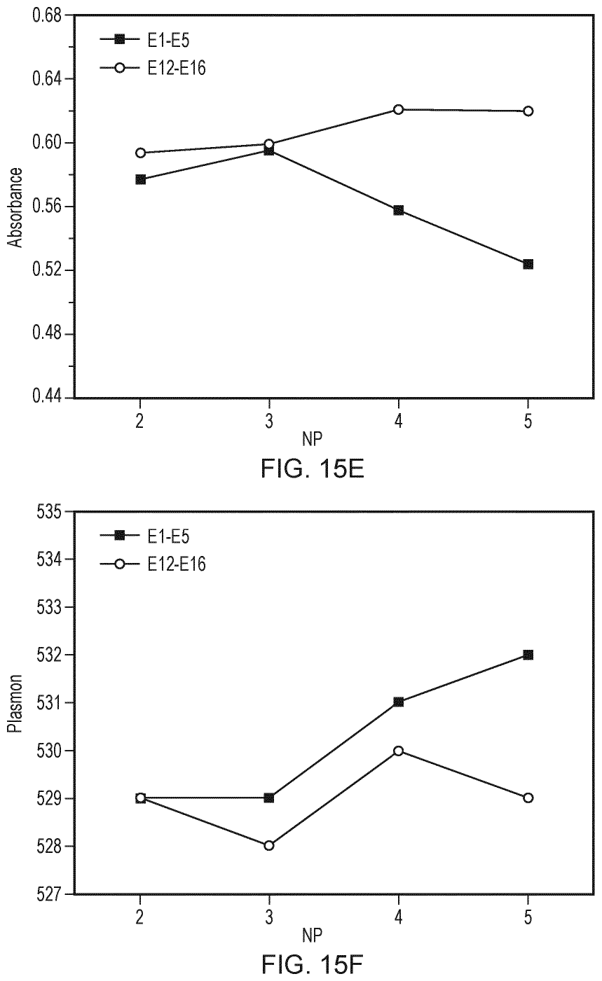

For the detection of 235 nt target sequence, two different sets of capture probes were employed: capture probes E1-E5 from the 5′ end to the middle of the sequence and capture probes E12-E16 from the middle of the 3′ end. The samples containing NPs (150 μM), target sequence 235 nt (1 nM) and NaCl 0.3 M were incubated for 48 hours (FIGS. 15A-C). These data suggested that systems containing 1 and 2 batches of gold nanoparticles remains invariant. For system containing 4 and 5 batches, the aggregation was more pronounced. Interestingly, the hybridization segment of capture probes with target sequence seems to be important for this type of detection strategy. While the aggregation takes place in the case of NPs stabilized with capture probes E1-E5, an effect was observed in NPs stabilized with E12-E16 (FIGS. 15D-F).

FIGS. 15D and 15E demonstrate that for capture probes E1-E5 the aggregation takes place for an assay containing more than two nanoparticles. Oppositely, for the capture probes E12-E16 there is no aggregation with increasing number of particles.

### Example 18—the Effect of Using Different Numbers of Batches on UV-Vis Spectra and Aggregation

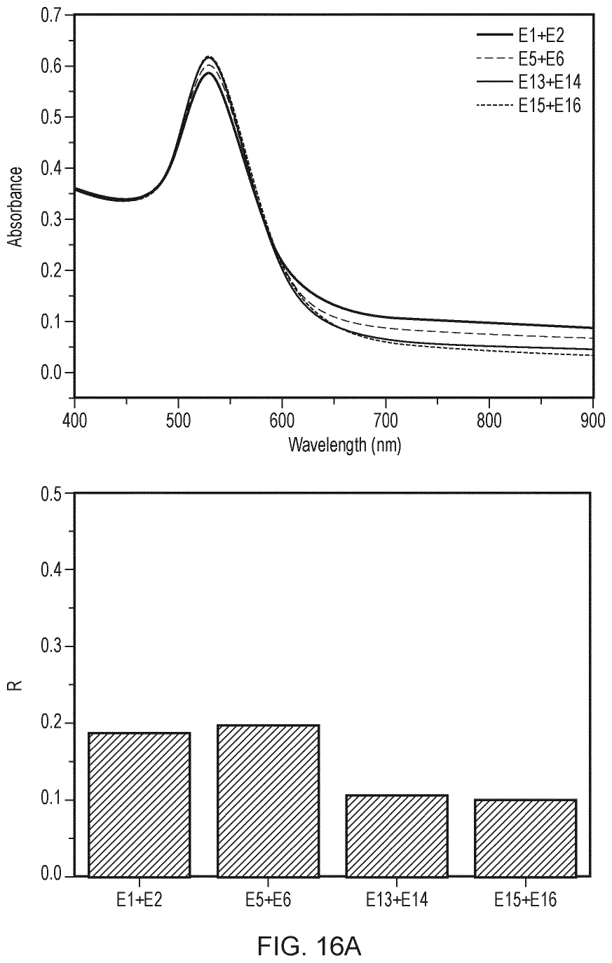

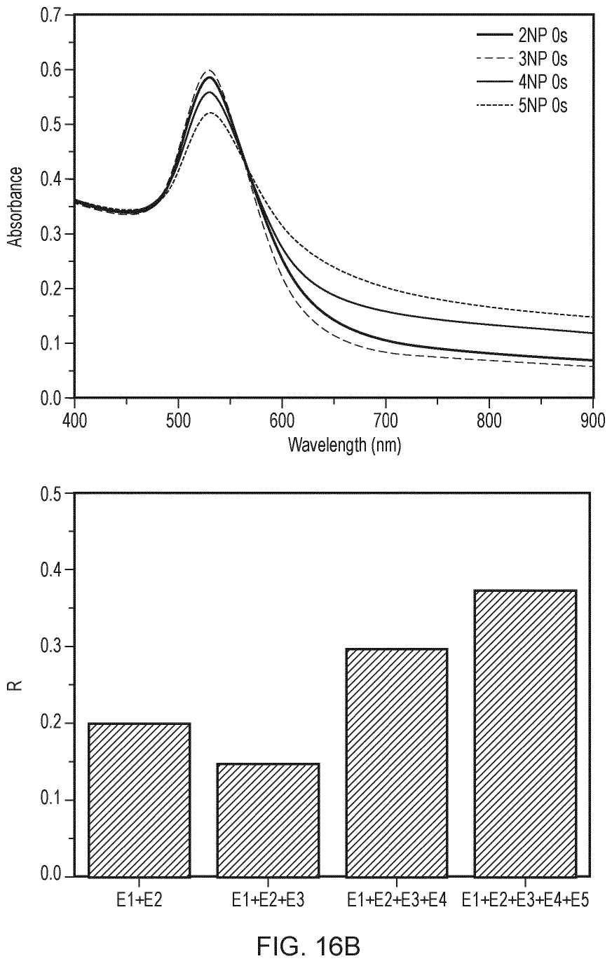

To demonstrate the importance of the number of particles employed in this type of aggregation assay, the assays were carried out using i) different combinations of 2 NPs (FIG. 16A) and ii) 3, 4 or 5 particles (FIG. 16B).

FIG. 16A shows UV Vis spectra and aggregation degrees for assays in which two batches of NPs were used. Four different combinations of nanoparticle populations which bound to different positions of the 235 nt viral analyte were used. FIG. 16B shows UV Vis spectra and aggregation degrees for assays in which two, three, four or five populations of nanoparticles were used. The conditions used in these assays were: target sequence 235 nt 1 nM, [Au] 150 uM, NaCl 0.3M.

These results demonstrate that the UV-Vis spectra is unchanged when only two populations of probes are used, regardless of the combination of probe sequence used. When four or five populations of probes are used, the UV-Vis spectra and the aggregation degree showed a clear change.

### Example 19—the Effect of Mismatches on Melting Temperature

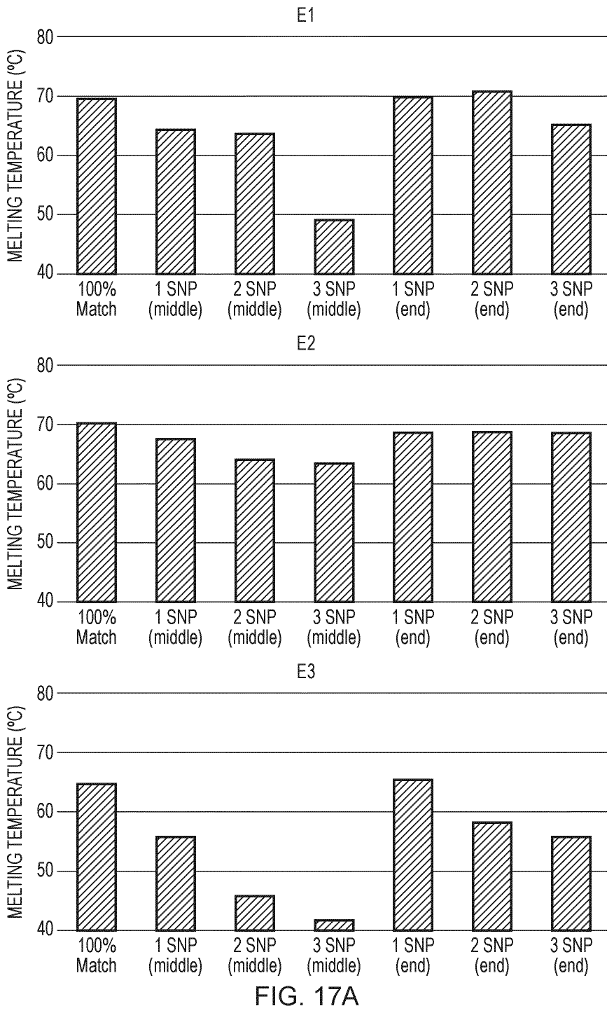

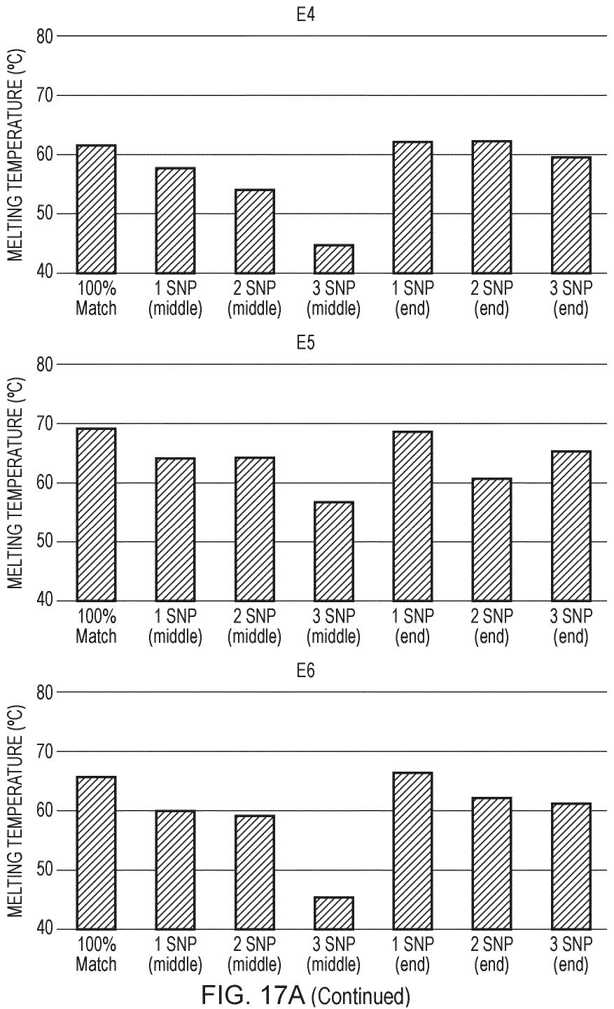

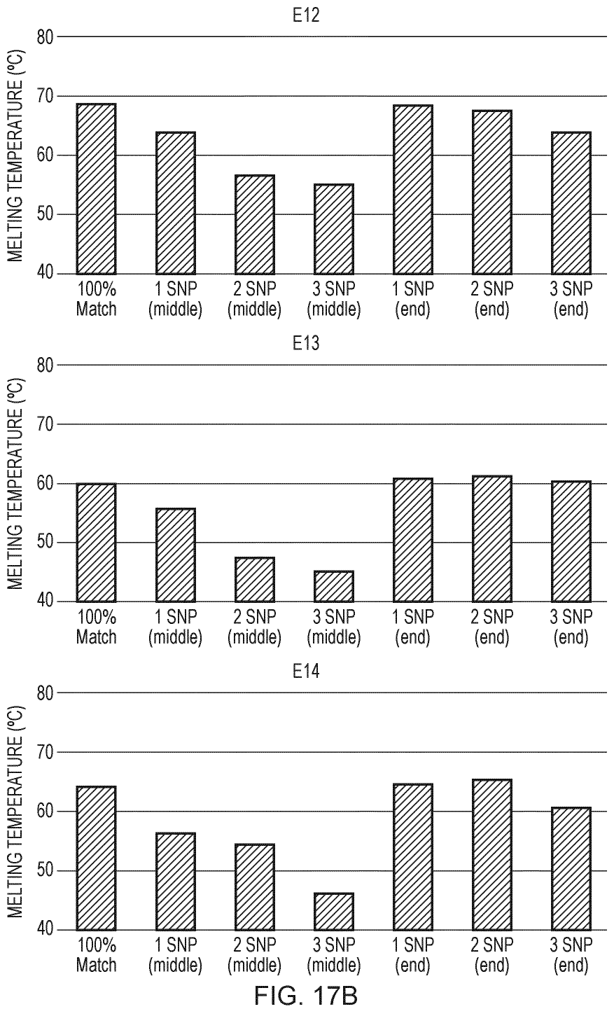

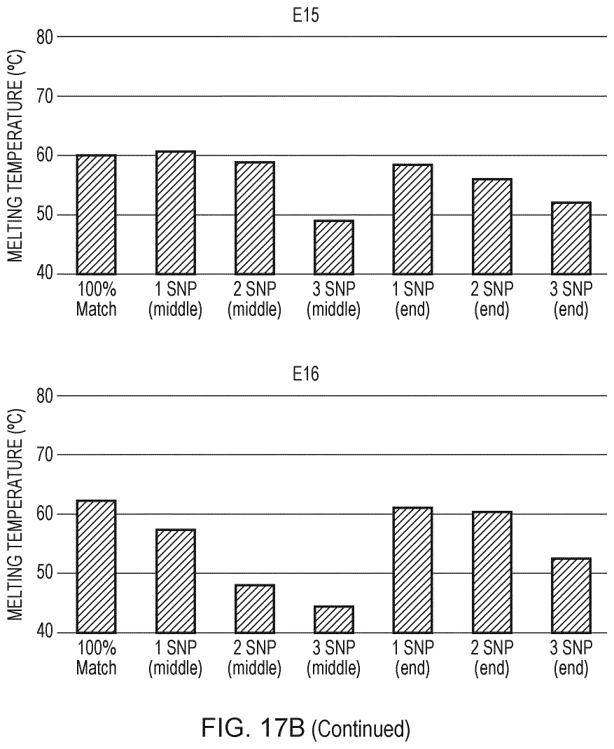

Melting curve calculations were performed for capture probe sequences E1-5, E7-11 (data not shown) and E12-16 according to 1, 2 or 3 nt changes in the sequence of their target analyte and according to position of change. The results of these experiments are provided in Table 5 and, for E1-E5 and E12-E16, FIGS. 17A and 17B respectively.

There was little change in binding affinity with 1 or 2 nt changes. 3 nt changes, which would account for 15% of the 20-mer sequence of the capture probe, were less tolerated. 3 nt changes were less tolerated when they occurred in a position on the target analyte that is complementary to the middle of the capture probe sequence.

### Example 20—Detection of Synthetic E Gene Analyte Using Non-Spherical Nanoparticles

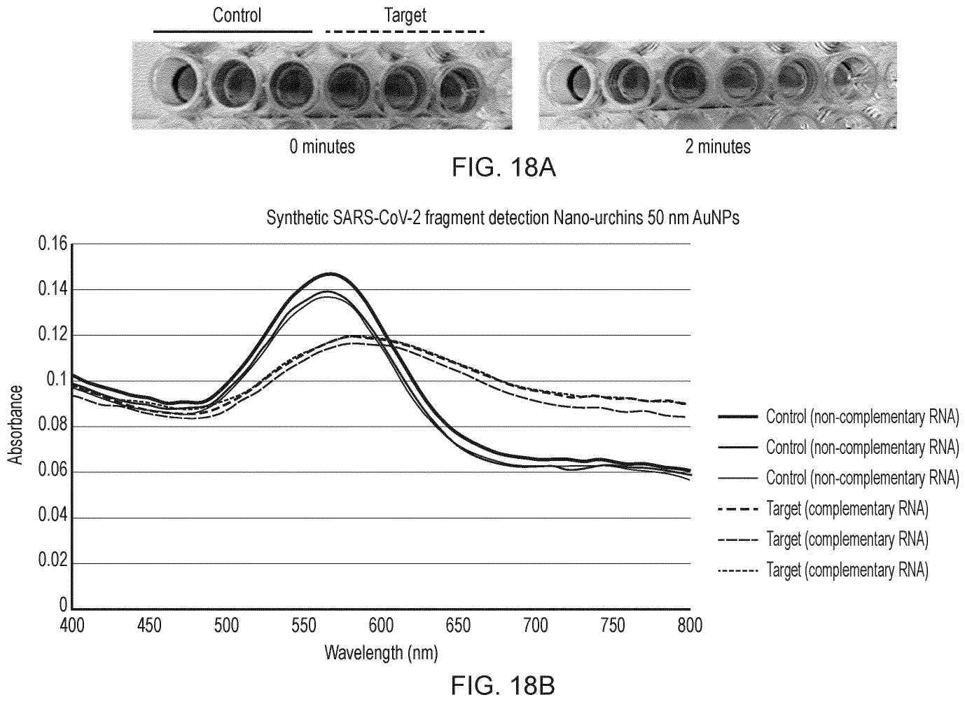

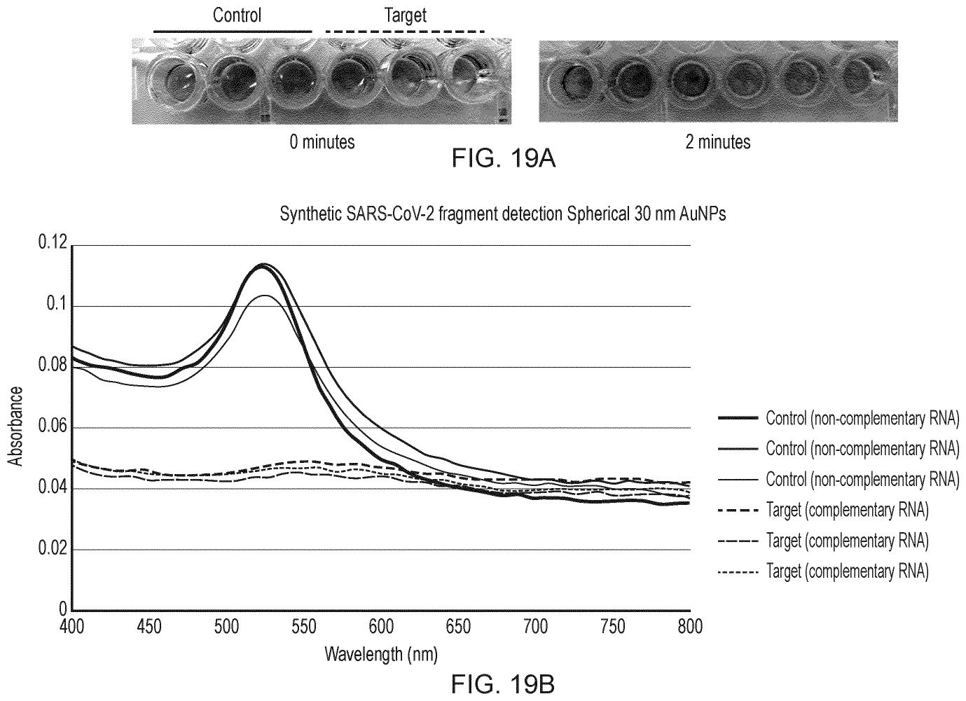

To determine whether detection of the SARS-CoV-2 E gene can be improved by using non-spherical nanoparticles, non-spherical 50 nm Au nano-urchins (sigma) (FIGS. 18A and B) or spherical 30 nm AuNPs (NanoComposix)(FIGS. 19A and B) were functionalized with three individual DNA probes (E9-E11) complementary to the sub-genomic E gene of SARS-CoV-2 and results were compared. The target analyte, the 3′ half of the E Gene (120 nt-long sequence of subgenomic RNA), was added at a concentration of 10 nM per well. The control wells contained a 108 nt-long RNA sequence that is non-complementary to the AuNP probes at a concentration of 10 nM per well. The colour change was detected by eye after two minutes and confirmed by UV-Vis spectra.

FIG. 18 shows that nano-urchins are able to specifically and rapidly detect the target analyte as illustrated by the clear colour change from pink to blue/transparent two minutes after adding the target analyte (FIG. 18A), corresponding to a clear spectral shift (FIG. 18B).

As opposed to the rapid colour change observed using nano-urchins, the colour change and spectral shift were less pronounced, but still adequate, using spherical nanoparticles (FIG. 19).

These results demonstrate that non-spherical nanoparticles can enhance the detection of the SARS-CoV-2 E gene.

### Example 21— Detection of Synthetic N Gene RNA with 30 nm Nanospheres

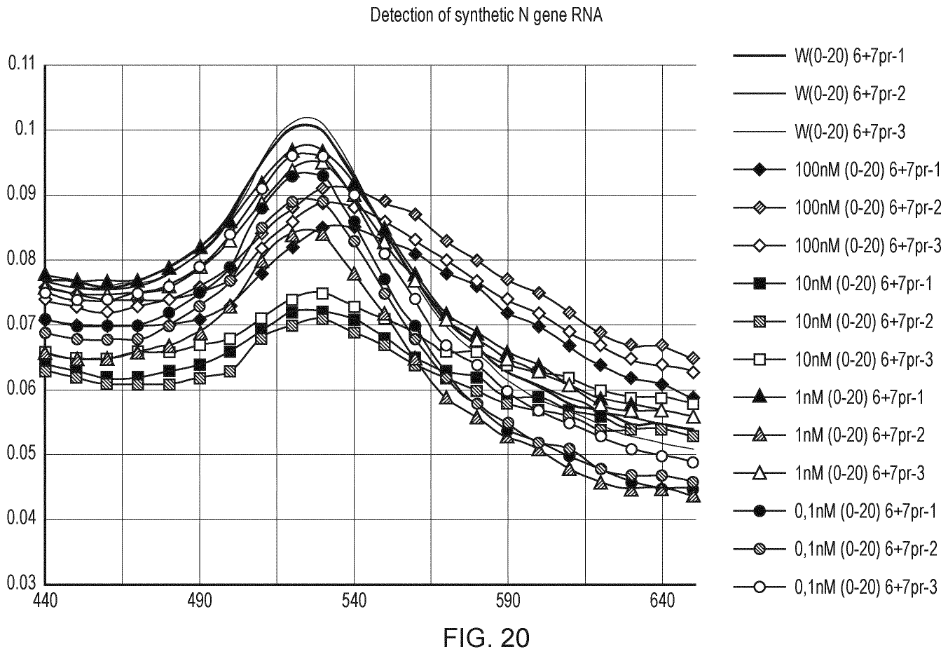

30 nm gold nanospheres (NanoComposix) were functionalised with a mix of seven (probes N1-7) or six probes (N8-20). The target analyte was a synthetically produced complete N gene sequence of SARS-CoV-2 (1260 nt)(FIG. 20). 1 μl of different concentrations, ranging from 100 nM to 0.1 nM, was employed in a total volume of 30 μl.

The results for this experiment indicate that 30 nm nanospheres functionalised with a mix of probes can detect long RNA sequences, in this case the N gene sequence of SARS-CoV-2. The experiment also demonstrates that nanoparticles can be functionalised with a mixture of multiple probes on the same nanoparticle, which advantageously provides for easier manufacturing of the nanoparticles.

### Example 22—SARS-CoV-2 Whole Genome Detection

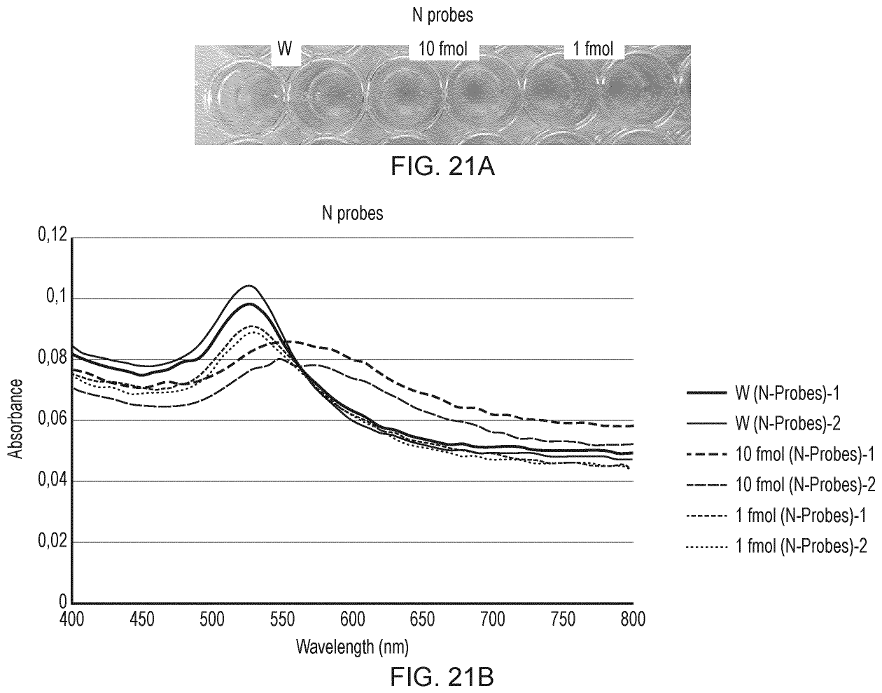

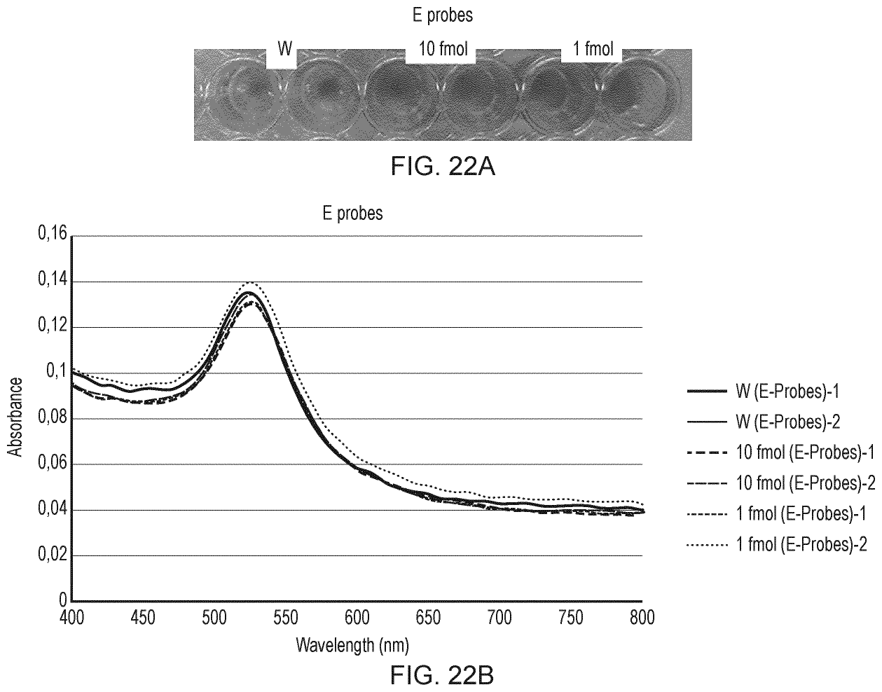

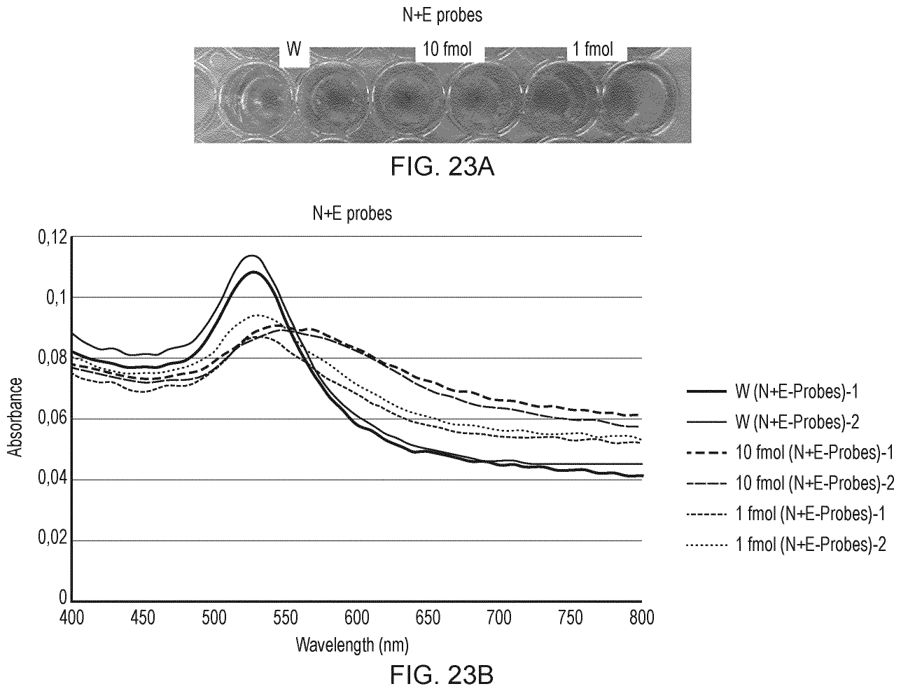

To test whether the Au NPs could be used to detect the whole genome of SARS-CoV-2, full-length genomic RNA (30,000 nt) was obtained from cell cultures of SARS-CoV-2-infected cells (Institute of Virology, Biomedical Research Centre of the Slovak Academy of Sciences, Slovakia) and used as target analyte. 30 nm gold nanoparticles were functionalised with either a mix of seven (probes N1-7) or six probes (N8-20)(FIG. 21), a mix of E gene probes (E7-17)(FIG. 22), or a mix of N and E gene probes (FIG. 23). The quantity of the target analyte added was 1 μl of 10 nM or 1 nM in a final volume of 30 μl. Detection of the target analyte was tested by determining whether there was a visible colour change after 25 and 120 minutes and this was confirmed by UV-Vis spectra.

The results of these experiments clearly show that the AuNPs allow for the detection of the full-length RNA sequence of SARS-Cov-2. Furthermore, these experiments show that it is possible to detect RNA directly obtained from the virus.

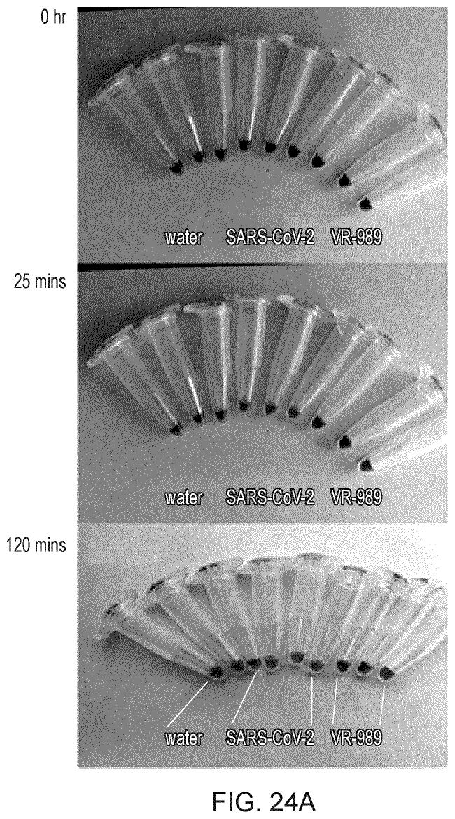

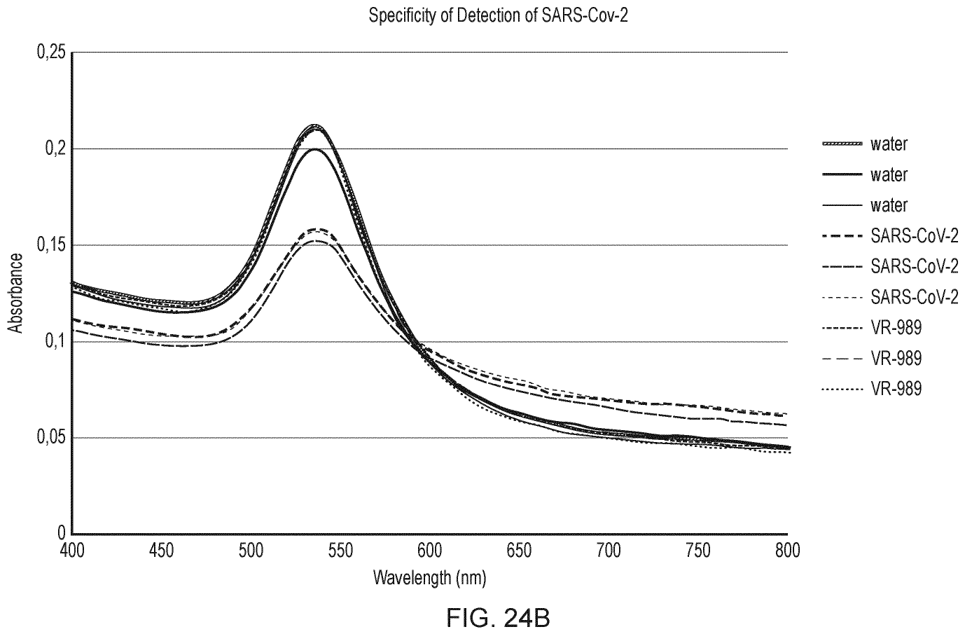

To demonstrate that the AuNPs detect SARS-CoV-2 specifically, a related coronavirus (feline alphacoronavirus VR-989) was used as target analyte and compared with SARS-CoV-2 (FIG. 24). 30 nm AuNPs were functionalized with a mix of E gene probes (E7-17) and 0.03 nM (1 μl of 1 nM in a total volume of 30 μl) of genomic RNA from SARS-CoV-2 or feline alphacoronavirus strain VR-989 (obtained from ATCC) were added as target analyte. Water was used as a control.

While the functionalised AuNPs were able to detect the SARS-CoV-2 after 25 minutes of incubation as demonstrated by the visible colour change (FIG. 24A), VR-898 did not cause a colour change or a spectral shift, even after 120 minutes of incubation (FIG. 24B).

This experiment demonstrates that the functionalised AuNPs of the present invention are specific for SARS-CoV-2 and do not detect closely related coronaviruses.

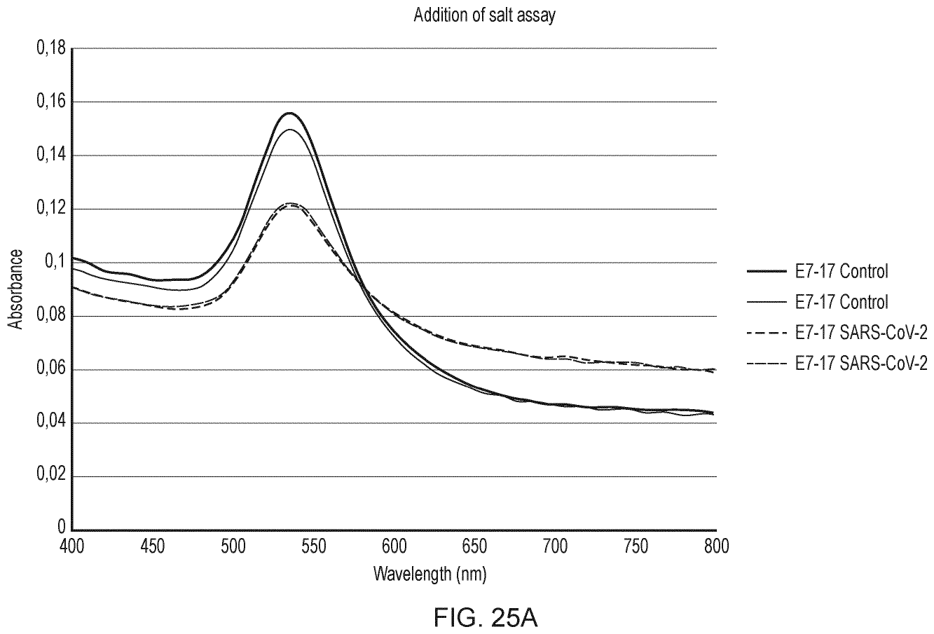

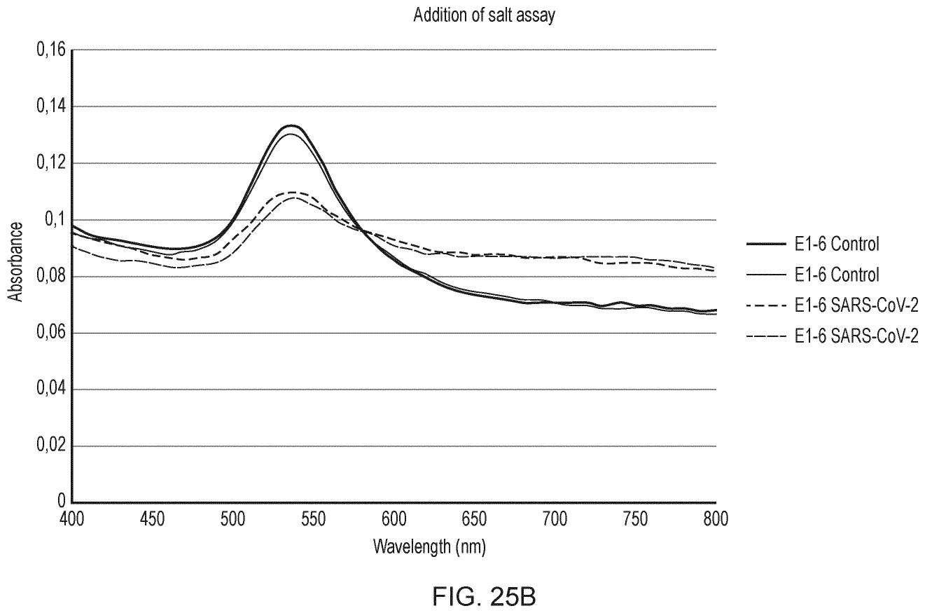

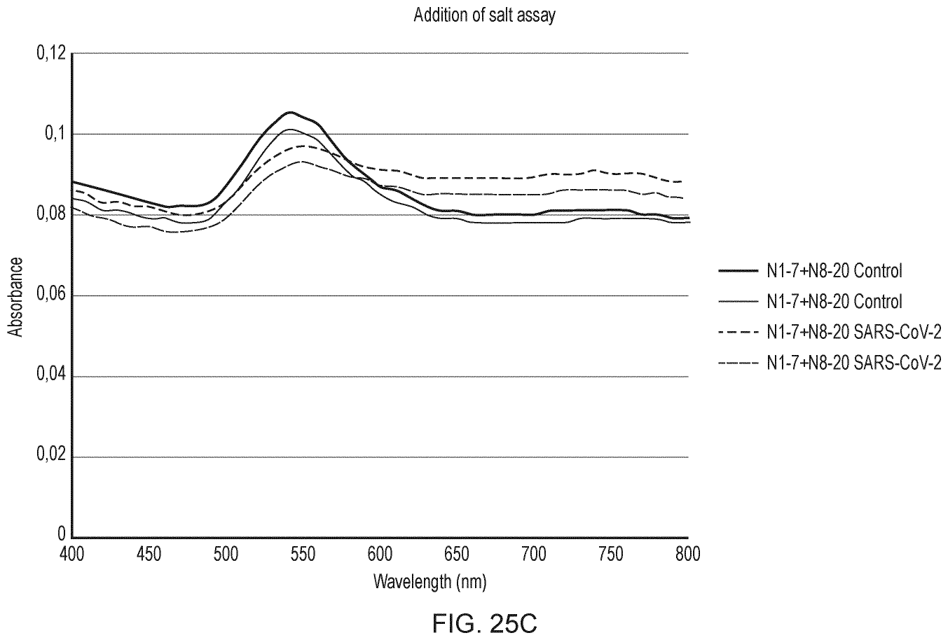

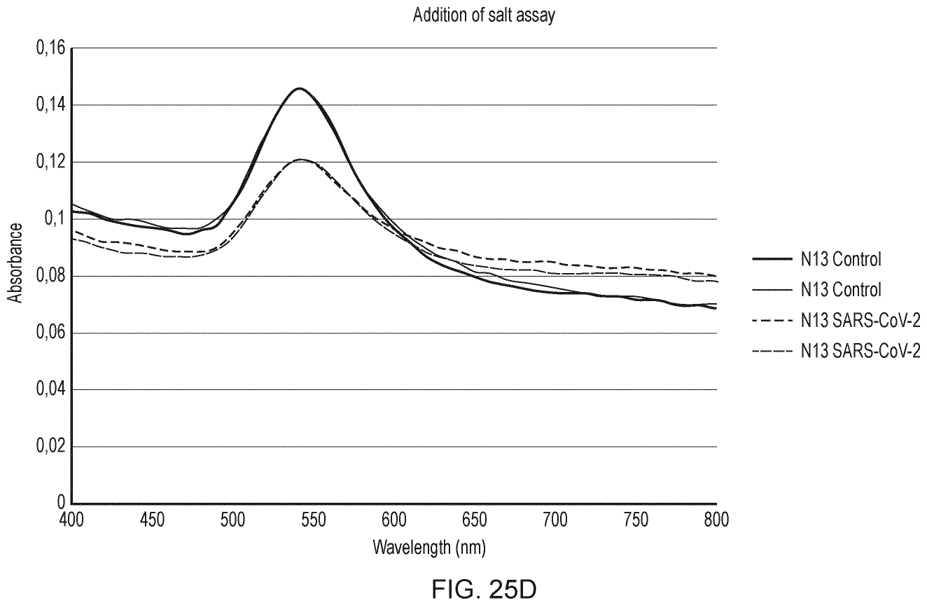

To enhance the speed of detection, extra salt was added to the assay after adding the AuNPs. Different mixes of probes: E gene: E7-17 (FIG. 25A) and E1-E6 (FIG. 25B); N gene: N1-N7 and N8-N20 (FIG. 25C), and N13 (FIG. 25D) were covalently attached to AuNPs, and then assayed with the full length RNA sequence of SARS-CoV-2 or with water as negative control. After adding the AuNPs, an extra 0.7M NaCl was added, reaching a final concentration of 1.5M. The addition of extra salt allowed for detection after only 20 minutes using either AuNPs functionalised with a mixture of probes (FIG. 25A-D), as demonstrated by a decrease in absorbance together with a clear spectral shift.

In conclusion, addition of extra salt after addition of the AuNPs improves the sensitivity of the assay, without affecting its specificity.

### Example 23—Improving the Limit of Detection (LOD)

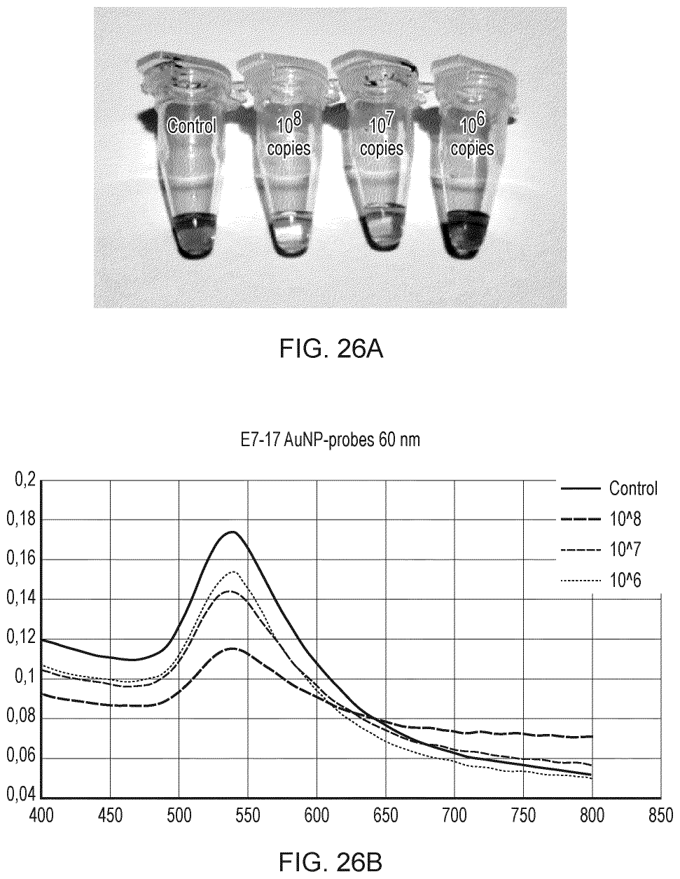

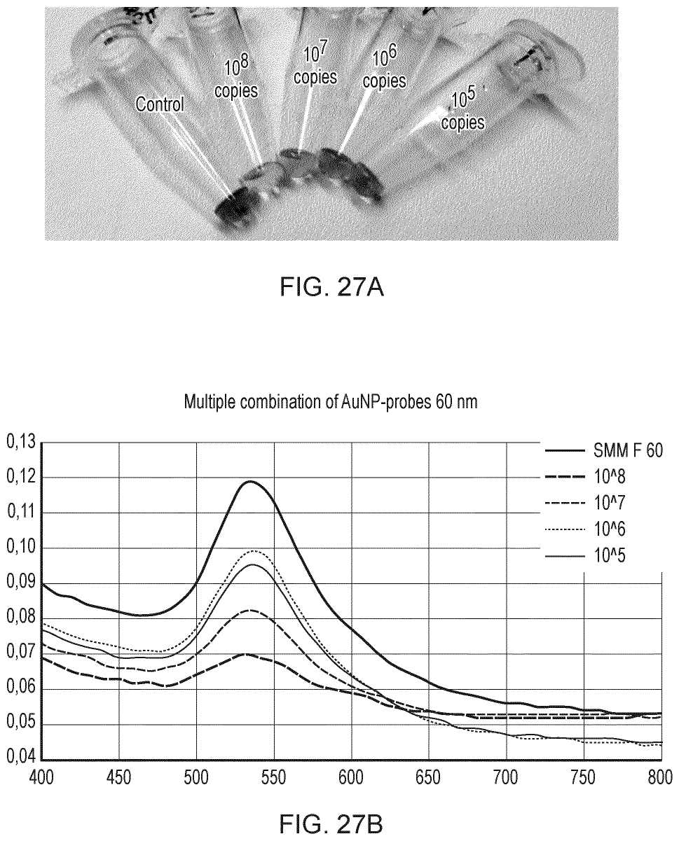

To determine the limit of detection, AuNPs were tested with a range of known concentrations of full-length genomic SARS-CoV-2 RNA containing: 10⁸ copies, 10⁷ copies, 10⁶ copies, and 10⁵ copies. AuNPs functionalised with probes E7-E17 resulted in a clear colour change at 10⁷ copies or more of virus after 15 minutes (FIG. 26). AuNPs functionalised with probes for E7-17 subgenomic (6), E1-5 genomic (5), N gene mix (10), and leader sequence probes (17) resulted in a clear colour change at 10⁵ copies or more of virus after 15 minutes (FIG. 27).

These data indicate that the AuNPs are capable of detecting concentrations as low as 10⁵ copies of target analyte.

### Example 24—Removing Protein from the Saliva Matrix

Previous experiments showed that the protein content of the saliva matrix was binding non-specifically to the nanoparticles rendering them unable to bind to the target analyte. It was therefore necessary to investigate ways of removing the protein from the matrix.

Proteinase K gave the best results. This coincided with information generated by Yale University that showed that a combination of proteinase K treatment with SDS could be used to directly inactivate the SARS-CoV-2 virus and extract RNA for use by qRT-PCR (https://publichealth.yale.edu/salivadirect/).

The use of SDS/Proteinase K avoids the need for a separate RNA extraction step and allows for a one-step detection method of target analyte. Unlike PCR, which requires the removal of both SDS and proteinase K before testing, SDS is a well-known stabiliser of NPs. PCR relies on the presence of enzymes, which requires the removal of proteinase K. Advantageously, there are no proteins in the present system, so removal of proteinase K is not necessary. Another advantage of using the SDS/Proteinase K method is that the SDS inactivates the virus, and a concentration of 0.5% SDS is sufficient to completely destroy the virus.

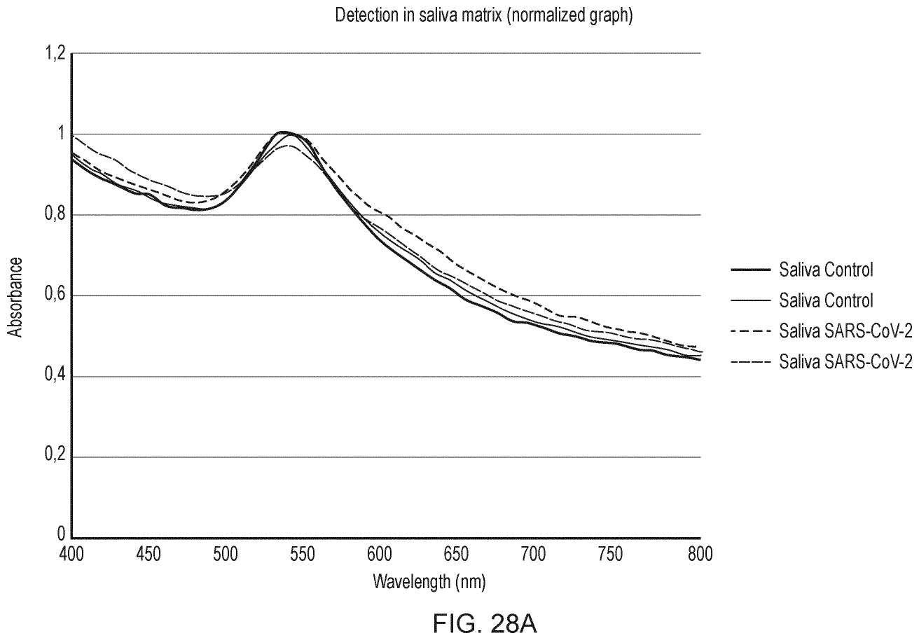

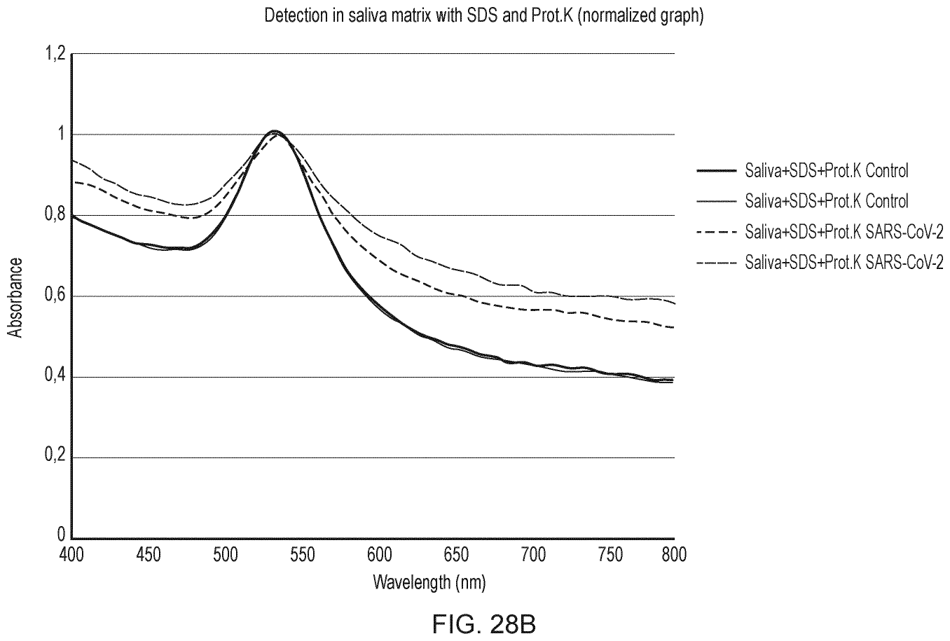

To test the SDS/Proteinase K treatment for use with the present assay, the inventors used a combination of 10 μg proteinase K and SDS at a final concentration of 0.5% (in powder form) and obtained similar results with saliva i.e. detection of SARS-CoV-2 RNA in <15 mins (FIG. 28). E7-17 AuNP-probes were employed in this experiment, and 1 μl of 260 μM of target analyte was used in a final volume of 65 μl.

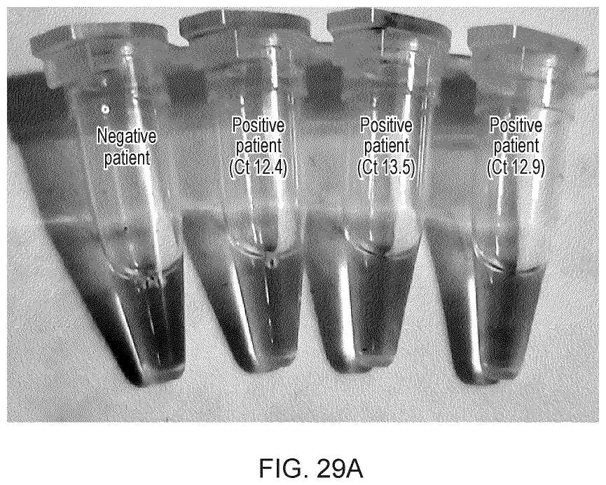

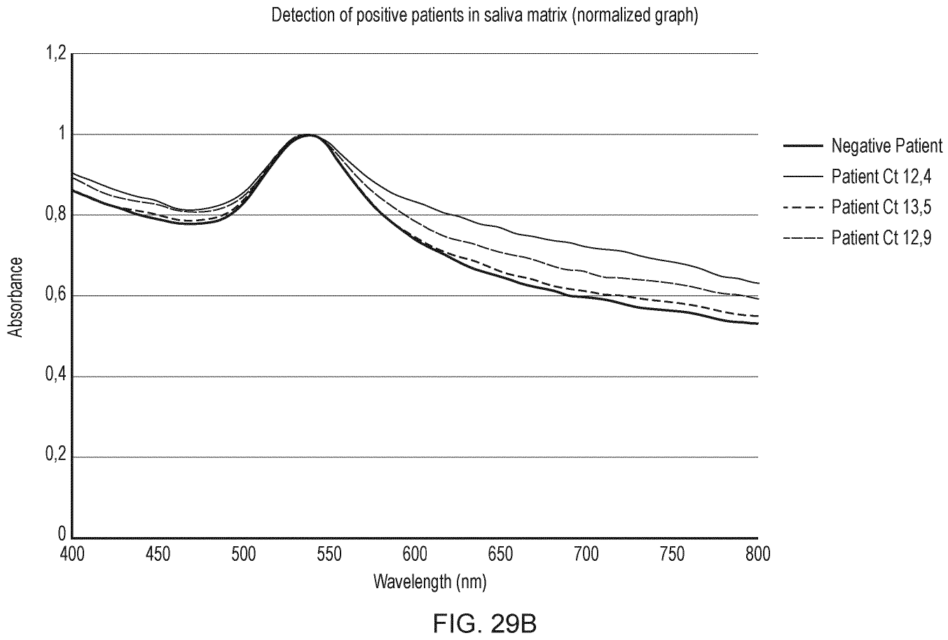

This experiment was repeated using patient-derived nasopharyngeal samples (RNA extracted for PCR) added to saliva (FIG. 29) which showed similar results.

In conclusion, these experiments demonstrate that adding SDS and proteinase K to the sample removes protein thus solving the problem of the protein content of the saliva interfering with the assay and allowing for the detection of viral RNA in patient-derived samples.

### Example 25—Clinical Testing of Individual Samples

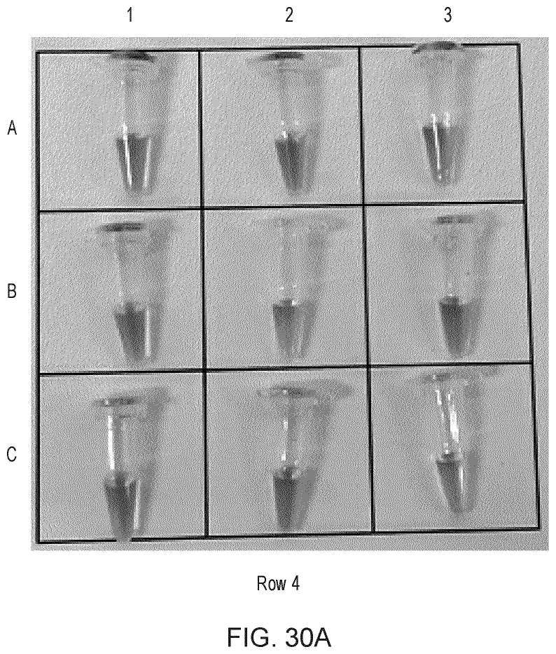

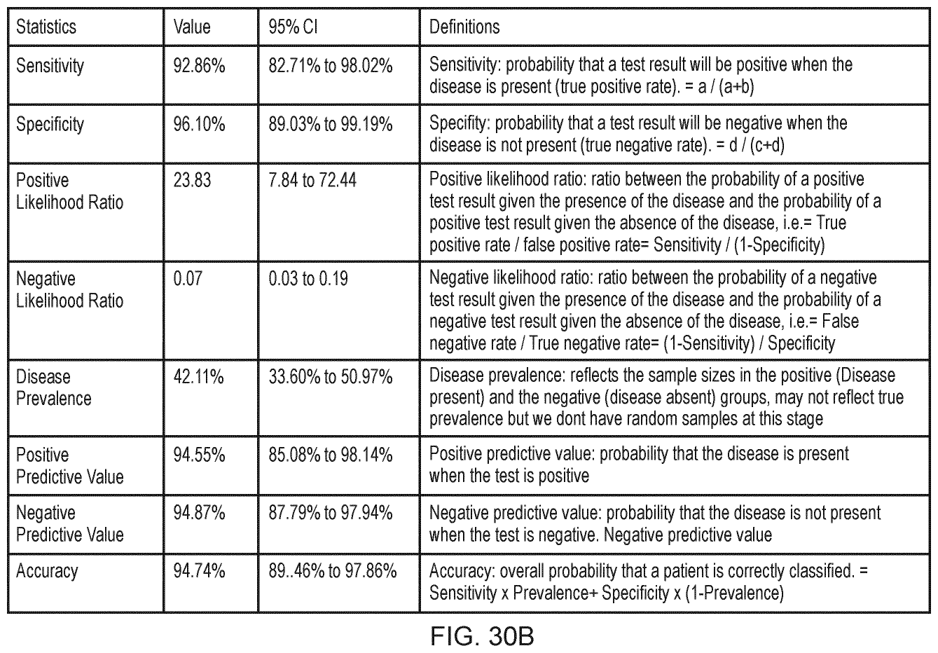

To determine whether the assay was able to detect SARS-CoV-2 in clinical samples, 175 nasopharyngeal clinical samples of known COVID-19 status, as tested by PCR, were obtained from the provincial (Gipuzkoa) COVID-19 testing centre. These were tested in a blinded fashion by six independent observers marking the tests as positive or negative by eye (FIG. 30). The consensus opinion was taken and the aggregated results calculated for clinical test parameters by Medcalc (FIG. 30B).

With a sensitivity of 92.86% and a specificity of 96.10%, the performance of the present invention lies between the standard PCR test and the lateral flow test. Lateral flow tests are capable of detecting 72% of people who are infected with the virus and have symptoms and 78% within the first week of becoming ill. But in people with no symptoms, that drops to 58% (ref 7). While more user-friendly than PCR tests, a major disadvantage of existing lateral flow tests is their poor sensitivity. PCR tests provide a greater sensitivity, but they lack scalability. The sensitivity of the present test is much better than the lateral flow test and only slightly below that of a PCR test. As such, the present invention advantageously provides for a convenient, user-friendly, test that can be mass-produced and has improved sensitivity compared to existing lateral flow tests.

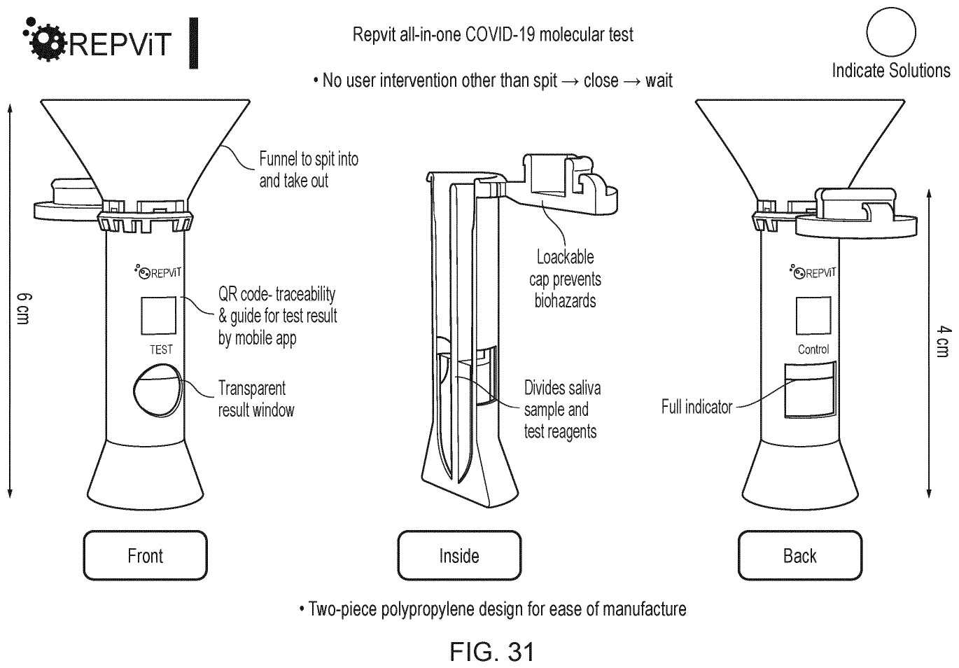

An example of a prototype of the present assay is provided in FIG. 31. The prototype comprises a tube containing the AuNPs and SDS/Proteinase, and a cap containing the extra salt. The user provides saliva into a funnel, closes the lid, adding the extra salt to the tube, and shakes the tube. After development, the results will be visible in the indicator window. A colour change from red to blue would be indicative of a positive SARS-CoV-2 test, whereas the absence of a colour change would be indicative of a negative test.

### Example 26—Use of Nanodiamonds as Nanoparticle

To increase the sensitivity of the invention further, the use of nanodiamonds as nanoparticles is contemplated herein. Nanodiamonds would be functionalised with thiolated oligonucleotide probes, and the presence of target analyte would lead to agglomeration of the nanodiamonds resulting in a change in luminescence, for example fluorescence. This change in luminescence is optically detectable and could be converted into a visual indicator/change.

TABLE 6
Melting curve calculations for different probe sequences according to 1, 2 or
3 nt changes in the sequence of the analyte and according to position of change

| | | | 100% Math | | | | | |
|---|---|---|---|---|---|---|---|---|
| | | Position | AG | Tm | 1 SNP (10^{a} base) | | 2 SNP (11^{a} base) | |
| | Length | Target | Kcal/mol | ° C. | AG | Tm | AG | Tm |
| E1 | 19 | 105-123 | −35.8 | 69.4 | −31.2 | 64.3 | −30.2 | 63.6 |
| E2 | 20 | 85-104 | −36.6 | 70.3 | −34 | 67.4 | −31.2 | 64.2 |
| E3 | 19 | 66-84 | −32.9 | 64.7 | −26.7 | 55.7 | −22.6 | 46 |
| E4 | 20 | 46-65 | −30.7 | 61.5 | −28 | 57.8 | −25.5 | 53.9 |
| E5 | 22 | 24-45 | −37.5 | 69.3 | −32.9 | 64.3 | −32.9 | 64.4 |
| E6 | 19 | 5-23 | −33.9 | 65.5 | −29.3 | 60.1 | −28.3 | 59.2 |
| E12 | 20 | 208-227 | −36.5 | 68.5 | −32.7 | 64 | −29.5 | 56.7 |
| E13 | 20 | 188-207 | −30.6 | 59.8 | −27.6 | 55.8 | −23.8 | 47.4 |
| E14 | 20 | 168-187 | −32.7 | 64.1 | −27.3 | 56.5 | −26 | 54.2 |
| E15 | 20 | 148-167 | −30.3 | 60 | −29.8 | 60.4 | −29.2 | 58.9 |
| E16 | 20 | 128-147 | −31.9 | 62.3 | −27.9 | 57.1 | −23.8 | 47.9 |

| | | | 100% Math | | | | | | | |
|---|---|---|---|---|---|---|---|---|---|---|
| | | Position | AG | Tm | 1 SNP (1^{a} base) | | 2 SNP (2^{a} base) | | 3 SNP (3^{a} base) | |
| | Length | Target | Kcal/mol | ° C. | AG | Tm | AG | Tm | AG | Tm |
| E1 | 19 | 105-123 | −35.8 | 69.4 | −35.5 | 70.1 | −36 | 70.8 | −31.6 | 65.3 |
| E2 | 20 | 85-104 | −36.6 | 70.3 | −35.9 | 69.1 | −34.4 | 68.9 | −35.2 | 68.8 |
| E3 | 19 | 66-84 | −32.9 | 64.7 | −33 | 65.5 | −28 | 58.1 | −26.4 | 55.7 |
| E4 | 20 | 46-65 | −30.7 | 61.5 | −30.7 | 62.4 | −30.6 | 62.4 | −28.2 | 59.8 |
| E5 | 22 | 24-45 | −37.5 | 69.3 | −37.9 | 68.7 | −33.2 | 60.7 | −33.9 | 65.7 |
| E6 | 19 | 5-23 | −33.9 | 65.5 | −34.1 | 66.4 | −30.2 | 62.1 | −29.1 | 61 |
| E12 | 20 | 208-227 | −36.5 | 68.5 | −36.3 | 68.2 | −34.1 | 67.3 | −31.9 | 63.8 |
| E13 | 20 | 188-207 | −30.6 | 59.8 | −30.7 | 60.7 | −30.6 | 61.1 | −30 | 60 |
| E14 | 20 | 168-187 | −32.7 | 64.1 | −32.4 | 64.7 | −32.8 | 65.4 | −28.7 | 60.6 |
| E15 | 20 | 148-167 | −30.3 | 60 | −28.7 | 58.4 | −26.6 | 55.9 | −24.8 | 51.9 |
| E16 | 20 | 128-147 | −31.9 | 62.3 | −31.2 | 61 | −30.1 | 60.5 | −25.1 | 52.5 |

### CONCLUSIONS

In summary, the results provided herein suggest that more than two batches of AU@DNA nanoparticles are required in mixture to detect targets of 108 nt and 235 nt. The method of the invention has enabled detection of the 235 nt target sequence. E1-E6 demonstrated optimal detection of the 235 nt target sequence. Furthermore, AU@DNA nanoparticles can specifically detect the full-length RNA sequence of SARS-Cov-2. The results further indicate that that the use of AU@DNA nanoparticles in an assay to detect SARS-CoV2 in patient samples provides for a more convenient test that can be mass-produced and has improved sensitivity compared to prior art tests.

All references cited herein are incorporated herein by reference in their entirety and for all purposes to the same extent as if each individual publication or patent or patent application was specifically and individually indicated to be incorporated by reference in its entirety.

The specific embodiments described herein are offered by way of example, not by way of limitation. Any sub-titles herein are included for convenience only, and are not to be construed as limiting the disclosure in any way.

### REFERENCES

1. Liu G, Lu M, Huang X, Li T, Xu D. Application of Gold-Nanoparticle Colorimetric Sensing to Rapid Food Safety Screening. Sensors (Basel). 2018;18(12):4166. Published 2018 Nov 27. doi:10.3390/s18124166
2. Armesto et al, Chapter 2 of Reverse Genetics of RNA Viruses: Applications and Perspectives (Wiley & Co. 2012).
3. Hurst, S J; Lytton-Jean, A K R; Mirkin, C A. “Maximizing DNA Loading on a Range of Gold Nanoparticle Sizes”. Anal Chem 2006, 78 (24), 8313-8318. https://doi.org/10.1021/ac0613582.
4. Corman V M, Landt O, Kaiser M, Molenkamp R, Meijer A, Chu D K W, et al. Detection of 2019 novel coronavirus (2019-nCoV) by real-time RT-PCR. Euro Surveill. 2020;25(3):2000045. https://doi.org/10.2807/1560-7917.ES.2020.25.3.2000045
5. Hofmann et al J Virol 64; 4108-4114
6. Moitra G, et al. Selective naked-eye detection of SARS-CoV-2 mediated by N gene targeted antisense oligonucleotide capped plasmonic nanoparticles. ACS Nano 2020; 14, 6, 7617-7627.
7. Dinnes J, Deeks J J, Berhane S, Taylor M, Adriano A, Davenport C, Dittrich S, Emperador D, Takwoingi Y, Cunningham J, Beese S, Domen J, Dretzke J, Ferrante di Ruffano L, Harris I M, Price M J, Taylor-Phillips S, Hooft L, Leeflang M M G, McInnes M D F, Spijker R, Van den Bruel A. Rapid, point-of-care antigen and molecular-based tests for diagnosis of SARS-CoV-2 infection. Cochrane Database of Systematic Reviews 2021, Issue 3. Art. No.: CD013705. DOI: 10.1002/14651858.CD013705.pub2
8. Miller, B. S., Bezinge, L., Gliddon, H. D. et al. Spin-enhanced nanodiamond biosensing for ultrasensitive diagnostics. Nature 587, 588-593 (2020). https://doi.org/10.1038/s41586-020-2917-1.
9. Torres Sangiao E, Holban A M, Gestal M C. Applications of Nanodiamonds in the Detection and Therapy of Infectious Diseases. Materials. 2019; 12(10):1639. https://doi.org/10.3390/ma12101639.
10. Chao J I, Perevedentseva E, Chung P H, Liu K K, Cheng C Y, Chang C C, Cheng C L. Nanometer-sized diamond particle as a probe for biolabeling. Biophys J. 2007 Sep 15;93(6):2199-208. doi: 10.1529/biophysj.107.108134.

1. A method for detecting the presence of a target nucleic acid analyte in a sample, wherein the target nucleic acid analyte comprises at least a first, a second and a third target sequence, which target sequences are spaced-apart or contiguous and non-overlapping, the method comprising:
a) providing a population of oligonucleotide probe-functionalised nanoparticles, said population comprising at least a first oligonucleotide probe that hybridizes to said first target sequence, a second oligonucleotide probe that hybridizes to said second target sequence and a third oligonucleotide probe that hybridizes to said third target sequence; and
b) contacting a solution comprising the sample with the population of nanoparticles, wherein multiple specific binding events between the oligonucleotide probe sequences and target sequences causes agglomeration of the nanoparticles, thereby resulting in a target nucleic acid analyte-dependent visible colour change of the solution.

2. The method of claim 1, wherein the population of nanoparticles comprises at least three, at least four, or at least five types of nanoparticle, wherein each type of nanoparticle is functionalised with multiple copies of one type of oligonucleotide probe.

3. The method of claim 1, wherein the population of nanoparticles comprises a plurality of different types of nanoparticle each functionalised with multiple copies of any of the at least three types of oligonucleotide probe.

4. The method of any one of claims 1-3, wherein the population of nanoparticles is functionalised with at least 4, 5, 6, 7, 8, 9, 10, 11, 12, 13, 14, 15, 16, 17, 18, 19, 20, 21, 22, 23, 24 or at least 25 types of oligonucleotide probe which hybridise to at least 4, 5, 6, 7, 8, 9, 10, 11, 12, 13, 14, 15, 16, 17, 18, 19, 20, 21, 22, 23, 24 or at least 25 corresponding spaced-apart or contiguous non-overlapping target sequences in the target analyte.

5. The method of any one of claims 1-4, wherein the population of nanoparticles is functionalised with 10 types of oligonucleotide probe which bind to 10 corresponding spaced-apart or contiguous non-overlapping target sequences in the target analyte.

6. The method of any one of claims 1-5, wherein one or more of the oligonucleotide probes is perfectly complementary to the target sequence to which it hybridises or comprises between one and three base mismatches.

7. The method of any one of the preceding claims, wherein the molar ratio of target to total nanoparticles is selected to permit target-specific nanoparticle agglomeration.

8. The method of any one of the preceding claims, wherein the molar ratio of target to total nanoparticles is in the range 1 to 0.001.

9. The method of any one of the preceding claims, wherein the method comprises a fragmentation step in which said target nucleic acid analyte is broken into two or more fragments.

10. The method of claim 9, wherein said fragmentation step comprises a period of sonication of the sample.

11. The method of claim 10, wherein the period of sonication is at least 10 seconds, at least 30 seconds or at least 60 seconds.

12. The method of any one of the preceding claims, wherein the target analyte comprises viral RNA.

13. The method of any one of the preceding claims, wherein the target analyte comprises viral genomic RNA, viral sub-genomic mRNA or viral mRNA.

14. The method of any one of the preceding claims, wherein the target analyte comprises SARS-CoV-2 RNA.

15. The method of any one of the preceding claims, wherein the target analyte comprises the genomic sequence of the E protein or the N protein of SARS-CoV-2.

16. The method of any one of the preceding claims, wherein the target analyte comprises the sgmRNA of the E gene or the N gene of SARS-CoV-2.

17. The method of claim 14, wherein the probe sequences are selected from SEQ ID NOs: 1, 2, 3, 4, and 5.

18. The method of claim 14, wherein the probe sequences are selected from SEQ ID NOs: 11, 12, 13, 14 and 15.

19. The method of claim 14, wherein the probe sequences are selected from SEQ ID NOs: 6, 7, 8, 9 and 10.

20. The method of any one of the preceding claims, wherein the nanoparticles have a core comprising metal or diamond.

21. The method of claim 20, wherein said core comprising metal comprises gold or silver.

22. The method of any one of the preceding claims, wherein the nanoparticles are spherical, non-spherical, ellipsoidal or bipyramidal.

23. The method of any one of the preceding claims, wherein the nanoparticles have a diameter between 13 nm and 65 nm.

24. The method of claim 18, wherein the mean diameter of the nanoparticles is between 20 nm and 60 nm, optionally wherein the mean diameter of the nanoparticles is about 30 nm.

25. The method of any one of the preceding claims, wherein the probes comprise DNA or a non-natural nucleic acid or a peptide nucleic acid (PNA).

26. The method of claim 25, wherein the probes comprise locked nucleic acid (LNA), 2′-H nucleic acid, 2′-OMe nucleic acid, or 2′-F nucleic acid.

27. The method of any one of the preceding claims, wherein nanoparticle-probe linkage is at the 5′ end of the oligonucleotide probes.

28. The method of any one of the preceding claims, wherein the probes comprise a thiol linkage to the nanoparticle surface.

29. The method of claim 28, wherein the probes comprise a C6-thiol 5′ linkage.

30. The method of any one of the preceding claims, wherein the probe sequences comprise between 10 and 100 nucleotides, optionally between 12 and 30 nucleotides.

31. The method of claim 30, wherein the target sequence hybridising portion of the probe sequences comprise 20 nucleotides.

32. The method of any one of the preceding claims, wherein the probes comprise a nucleotide tail upstream of the target sequence hybridising portion of the probe sequence.

33. The method of claim 32, wherein the nucleotide tail comprises 10 nucleotides.

34. The method of claim 32 or claim 33, wherein the nucleotide tail comprises a poly-T sequence, optionally a T₁₀ sequence.

35. The method of any one of the preceding claims, wherein the spacing between adjacent nanoparticles when the probes are hybridised to the target sequences of the target analyte is between 5 nm and 30 nm.

36. The method of claim 35, wherein the spacing between adjacent nanoparticles when the probes are hybridised to the target sequences of the target analyte is between 10 nm and 18 nm.

37. The method of claim 36 wherein the spacing between adjacent nanoparticles when the probes are hybridised to the target sequences of the target analyte is 12 nm.

38. The method of any one of the preceding claims, wherein the method further comprises a step of adding Sodium Dodecyl Sulfate (SDS) and proteinase K.

39. The method of claim 38, wherein the final concentration of SDS is at least 0.5%.

40. The method of any one of the preceding claims, wherein the method further comprises a step (c) of adding extra salt.

41. The method of any one of the preceding claims carried out at less than 45° C.

42. The method of claim 41, wherein the method does not involve addition of RNAse.

43. A population of oligonucleotide probe-functionalised nanoparticles comprising at least a first, a second and a third oligonucleotide probe which hybridise to at least a first, a second and a third spaced-apart or contiguous and non-overlapping target sequence in a target nucleic acid analyte.

44. The population of oligonucleotide probe-functionalised nanoparticles of claim 43 for use in a method as defined in any one of claims 1-42.

45. The population of oligonucleotide probe-functionalised nanoparticles of claim 43 or claim 44, wherein said nanoparticles are as defined in any one of claims 1-42.

46. A kit for detection of a target nucleic acid analyte in a sample, the kit comprising:
a population of nanoparticles as defined in any one of claims 43-45;
a reaction vessel for holding a solution, the reaction vessel comprising at least a wall and a sealable opening, wherein visible light is able to pass through at least a portion of the wall and/or the sealable opening; and
one or more reagents or solutions for carrying out the method of any one of claims 1-42.

47. The kit of claim 46, further comprising one or more reagents or solutions for isolating target nucleic acid from a sample.

48. The kit of claim 46 or claim 47, further comprising a colour reference corresponding to the expected colour change upon positive detection of the target nucleic acid analyte.

49. A device for detecting the presence of a target nucleic acid analyte in a sample, the device comprising:
an inlet for receiving a sample,
a passage for the sample to pass from the inlet to a storage compartment pre-loaded with a set of reagents,
wherein the reagents comprise the population of nanoparticles as defined in claims 43-45.
and a detection window comprising a colour reference, wherein, in use, the sample contacts the reagents in the storage compartment, so that the positive detection of a target nucleic acid analyte results in an expected colour change in the colour reference visible through the detection window.

50. The device of claim 49, wherein the sample is a saliva sample.

51. The device of claims 49 to 50, further comprising a cap.

52. The device of claim 51, wherein the cap comprises NaCl, such that, upon closing the cap, the salt is added to the storage compartment.

53. The device of any one of claims for use in a method as defined in claims 1-42.
